(19) United States
(12) Patent Application Publication
(10) Pub. No.: US 2024/0284863 A1
(43) Pub. Date: Aug. 29, 2024

(54) MICROBIAL TOOLS FOR IMPARTING BENEFICIAL PROPERTIES TO SEAWEED CULTIVARS

(71) Applicant: University of Southern California, Los Angeles, CA (US)

(72) Inventors: Sergey Nuzhdin, Los Angeles, CA (US); Melisa Osborne, Los Angeles, CA (US); Gary Molano, Los Angeles, CA (US); Bernadeth Tolentino, Los Angeles, CA (US)

(21) Appl. No.: 18/589,339

(22) Filed: Feb. 27, 2024

Related U.S. Application Data
(60) Provisional application No. 63/449,016, filed on Feb. 28, 2023.

(51) Int. Cl. A01H 13/00; A01D 44/00; A01G 33/00; C12N 1/20

(52) U.S. Cl. CPC A01H 13/00; A01D 44/00; A01G 33/00; C12N 1/20

(57) ABSTRACT

This disclosure describes the microbial network and community assembly patterns of brown macro algae, e.g., Macrocystis pyrifera gametophyte germplasm, cultures used to cultivate an offshore farm and identify network features associated with increased biomass of mature sporophytes. Several network features, such as clustering coefficient and edge ratios, significantly vary with biomass outcomes; also, gametophytes that become low- or high biomass sporophytes have different hub taxa. This disclosure describes microbial community dynamics in M. pyrifera germplasm cultures and development of early life stage inoculants used on seaweed cultivars to increase biomass yield.

### CROSS-REFERENCE TO RELATED APPLICATIONS

This application claims benefit of priority under 35 U.S.C. § 119(e) of U.S. Ser. No. 63/449,016, filed Feb. 28, 2023, the contents of which are incorporated herein by reference in its entirety.

### STATEMENT OF GOVERNMENT SUPPORT

This invention was made in part with government support under Grant no. GR1022773, awarded by the (ARPA-E) Advanced Research Projects Agency—Energy; Grant no. T32-GM118289, awarded by the (NIH/NIGMS) National Institute of General Medical Sciences; and Grant no. 00001567, awarded by the (EPA) U.S. Environmental Protection Agency. The government has certain rights in the invention.

### BACKGROUND OF THE INVENTION

### Field of the Invention

This invention relates to isolated and biologically pure microbes and microbial consortia that are useful for imparting beneficial properties to seaweed cultivars. These microbes or their microbial consortia can be formulated into agriculturally acceptable compositions.

### Background Information

With national interest in seaweed-based biofuels as a sustainable alternative to fossil fuels, there is an unmet need for tools that impart beneficial properties—including but not limited to high biomass, increased yield, improved nitrogen fixation, improved resistance to temperature stress or improved resistance to disease—to seaweed cultivars, in order to increase the efficiency of offshore seaweed farms.

Seaweed aquaculture, the harvest of wild and farmed seaweeds, is a global industry that in 2018 was valued at over $13 billion USD. Seaweed farming not only produces biomass to support commercial interests such as the pharmaceutical, food, and cosmetic industries, but also improves ocean conditions by sequestering excess nutrients and carbon to reduce eutrophication and ocean acidification. This industry is now being targeted in the U.S. as a viable and sustainable resource for biofuel feedstocks that replace the use of fossil fuels and reduce carbon emissions. In fact, the Department of Energy Advanced Research Projects Agency-Energy (ARPA-E) has invested over $60 million through programs such as the Macroalgae Research Inspiring Novel Energy Resources to fund projects and research that advance seaweed aquaculture in the U.S. for this purpose. To support this effort, the National Oceanic and Atmospheric Administration (NOAA) has identified Southern California as a prime location for developing the aquaculture industry.

Macrocystis pyrifera, a brown macroalgae commonly known as “giant kelp,” is a candidate for biofuel production in Southern California. It is native to the California coastline and doubles its biomass every 34 d. A majority of biofuel consumed in the U.S. is produced by blending petroleum gasoline with ethanol extracted from renewable resources, such as seaweeds, and is typically used for transportation. The high carbohydrate and low lignin content of M. pyrifera make it an ideal feedstock for producing ethanol through fermentation, with an ideal yield estimated to be ˜0.281 weight (wt) ethanol·wt biomass⁻¹. Anaerobic digestion is also performed on M. pyrifera to produce biogas, which is used as a replacement for natural gas for heating and electricity.

Macrocystis pyrifera sporophytes (adults) propagate by releasing spores that develop into male and female gametophytes. This haplodiplontic life cycle of M. pyrifera enables individual genotypes to be clonally and vegetatively propagated and maintained in a gametophyte germplasm bank, which provides a library of genetic diversity that supports robust crop lines and serves as a reliable source material for farms. Targeted crosses of specific genotypes in this germplasm are repeatedly crossed towards specific phenotypic targets, such as increased biomass. The germplasm was used to create a breeding program for M. pyrifera that results in high biomass individuals that thrive in an offshore farm off the coast of Santa Barbara.

Microbial inoculation, the application or introduction of beneficial bacteria to plants, has been explored to improve the fitness of crops in agriculture. There are several successful examples of inoculants increasing the yield of agricultural crops including rice, peanut, maize, cotton, lavender, wheat, tomato, and spinach. In fact, inoculating seeds with growth-promoting bacteria increases farm productivity and the composition of seed microbiomes is an early predictor of final yield. Seed microbiota impact plant performance through priority effects, meaning that the first taxa to colonize a host influences successive community structure, function, and host-microbe associations throughout the rest of the plant life cycle. The overall fitness and resilience of seaweeds are partly regulated by resident microbial communities that provide services for normal growth and development, such as scavenging for growth-limiting nutrients or providing secondary metabolites. There is no published work investigating the microbiome of early life stage farmed seaweeds (i.e., gametophyte cultivars) and its impact on final biomass yield.

Building off the knowledge that microbes have an impact on plant health, there has been a wealth of research on the use of microbial inoculants (i.e., the introduction or addition of beneficial bacteria to a host) in agriculture. Addition of growth-promoting bacteria increases the fitness and yield of several agricultural crops including rice, maize, and cotton. In particular, the use of these inoculants at an early life stage in plant hosts increases farm productivity and predicts yield. Many studies focus on the impact that individual microbes have on host health rather than the indirect role of microbe-microbe interactions and microbial community dynamics. While useful, this approach is limited because the variability of microbe-microbe and microbe-plant interactions across hosts means that an inoculant with the desired effect on one plant host will not have the same effect on all plant hosts. Indeed, host microbiomes are not a collection of inert individuals, but rather an interdependent community with complex functional and metabolic pathways. Microbial inoculants compete with native species, preventing successful colonization of the inoculant or causing negative impacts on crop performance. Inoculants also prompt microbial succession, thereby altering community structure and function. As a result, insufficient consideration of the existing microbial network and community dynamics when designing inoculants contributes to low efficacy during large-scale applications in agriculture. Therefore, in order to fully harness the beneficial impact of microbes and establish a strong framework for growth-promoting inoculants, it is important to develop inoculants through manipulation of microbial community dynamics.

### SUMMARY OF THE INVENTION

The present disclosure provides for an efficient and broadly applicable agricultural platform utilizing microbes and microbial consortia that impart one or more beneficial properties in seaweed.

In one embodiment, the present disclosure relates to a method of obtaining harvested seaweed having at least one desired characteristic including: a) contacting a seaweed spore with a culture media including an inoculum, wherein the inoculum includes an effective amount of at least one microbe; b) growing the spore into a gametophyte; c) outplanting the gametophyte; and d) harvesting the seaweed. In some aspects, the harvested seaweed has at least one desired characteristic that distinguishes the harvested seaweed from non-inoculated seaweed, selected from high biomass, increased growth rate, increased yield, increased stress tolerance, increased resistance to disease, increased pathogen resistance, increased photosynthetic rate, increased tolerance of heavy metals, apical cell growth rate, disease resistance, apical cell multiplication rate, root length, root nodulation, holdfast strength, holdfast morphology, blade length, stalk length, pneumatocyst size, pneumatocyst number, mineral content, chelation of micronutrients, heat tolerance, cold tolerance, or chlorophyll content. In one aspect, the at least one desired characteristic is high biomass. In another aspect, the non-inoculated seaweed is selected from a parent sporophyte, a sporophyte of the same variant, a sporophyte of the same gametophyte culture, a sporophyte of the same cultivar, or a sporophyte of the same genotype. In some aspects, the inoculum includes an effective amount of at least one microbe isolated from a seaweed, one or more spores from a seaweed, or one or more seaweed gametophytes, or any combination thereof. In one aspect, the inoculum includes an effective amount of at least one microbe isolated from a high biomass seaweed, one or more spores from a high biomass seaweed, or one or more gametophytes from a high biomass seaweed, or any combination thereof. In one aspect, the at least one microbe is a kelp-associated microbe. In one aspect, the kelp-associated microbe is selected from Marinomonas brasilensis, mucus bacterium 80, Zoogloea ramigera, Mesorhizobium species, Nitratireductor species, Aquamicrobium species, Pedobacter species, Wenyinzhuangia species, species from the order Desulfovibrionales, species from the order Nitrospinales, species from the family Magnetococcaceae, species from the family Beijerinckiaceae, species from the family Holosporaceae, species from the family Brucellaceae, species from the order Sphingomondales, and species from the order Thermoanaerobacterales; or any combination thereof. In one aspect, the inoculum includes at least one microbe selected from Mesorhizobium species, Wenyingzhuangia species and Pedobacter species; or a variant thereof. In another aspect, the inoculum includes at least one Mesorhizobium species or a variant thereof. In various aspects, the inoculum optionally does not contain species from the genus Labrenzia. In certain aspects, the culture media includes agriculturally acceptable excipients. In one aspect, the seaweed is Macrocystis pyrifera or a variant thereof. In one aspect, the seaweed spore in b) is a Macrocystis pyrifera spore or a variant thereof. In an additional aspect, the culture media includes sterile Provasoli enriched seawater medium.

In one embodiment, the disclosure provides an agricultural formulation for an inoculant including a mixture of two or more bacterial species selected from Marinomonas brasilensis, mucus bacterium 80, Zoogloea ramigera, Mesorhizobium species, Nitratireductor species, Aquamicrobium species, Pedobacter species, Wenyinzhuangia species, species from the order Desulfovibrionales, species from the order Nitrospinales, species from the family Magnetococcaceae, species from the family Beijerinckiaceae, species from the family Holosporaceae, species from the family Brucellaceae, species from the order Sphingomondales, or species from the order Thermoanaerobacterales. In one aspect, the agricultural formulation further includes at least one agriculturally acceptable excipient. In an additional aspect, the agricultural formulation does not contain species from the genus Labrenzia. In various aspects, the agricultural formulation optionally contains species from the genus Labrenzia.

In one embodiment, the disclosure provides a method of making an inoculant including: a) collecting a sporophyte from a defined geographical region; b) collecting a spore from the sporophyte; c) analyzing at least one microbe obtained from the sporophyte; d) growing a mature sporophyte from the spore; e) identifying the mature sporophyte as a high biomass sporophyte; f) culturing a microbial community of the high biomass sporophyte in biomass media; and g) adding at least one agriculturally acceptable excipient to the biomass media to make an inoculant. In one aspect, the high biomass sporophyte is a brown macro algae, e.g., Macrocystis pyrifera sporophyte.

In one embodiment, the disclosure provides a method of obtaining a high biomass seaweed including incubating at least one spore or gametophyte with a culture media including the inoculant described herein.

In one embodiment, the present disclosure provides a method of obtaining high biomass seaweed including: a) releasing a spore from a sporophyte in a culture media; b) identifying at least one bacterial species present in the culture media; c) determining that the at least one bacterial species is associated with a high biomass seaweed; d) growing the spore into a gametophyte; e) outplanting the gametophyte; and f) harvesting a high biomass seaweed. In one aspect, the high biomass seaweed is Macrocystis pyrifera. In certain aspects, the culture media is sterile Provasoli enriched seawater medium. In some aspects, determining that the at least one bacterial species is associated with a high biomass seaweed includes modeling network features of a microbiome of the spore to determine that the bacterial species is associated with a high biomass seaweed. In various aspects, the network features used are chosen from clustering coefficients, positive to negative edge ratios, modularity, heterogeneity or average path length.

In one aspect, the inoculum is obtained by: a) swabbing blades of a sporophyte to collect a sample; b) verifying the presence of microbes in the sample; c) culturing the microbes to form a microbial community; and d) adding an agricultural excipient to the microbial community to form an inoculum. In another aspect, the inoculum is obtained by: a) releasing a spore from the first sporophyte; b) identifying microbes from the first sporophyte and from the spore; c) producing a second sporophyte from the spore; d) confirming the second sporophyte is a high biomass seaweed; e) culturing the microbial community; and f) adding an agricultural excipient to the microbial community to form an inoculum. In some aspects, the inoculum is obtained by: a) measuring biomass of a sporophyte; b) collecting a microbial community from the sporophyte; c) quantifying diversity of microbial taxa in the microbial community; d) calculating and recording clustering coefficient values of the microbial community at multiple taxonomic levels, wherein the calculating and recording clustering coefficient values comprises at least one of; i) identifying the microbial community as having a large clustering coefficient value at the species level; ii) identifying the microbial community as having few hub taxa with dense connections at the order and/or family level; e) culturing the microbial community; and f) adding an agricultural excipient to the microbial community to form an inoculum.

### BRIEF DESCRIPTION OF THE DRAWINGS

FIG. 1 is a schematic diagram illustrating workflow for data collection and network construction. In step 1, wild M. pyrifera sporophylls were collected from four natural populations across Southern California: Arroyo Quemado (AQ), Catalina Island (CI), Camp Pendleton (CP), and Leo Carillo (LC). In step 2, reproductive blades were surface sterilized and prepared for spore release. In step 3, spores were released in sterile Provasoli enriched seawater medium (PES). In step 4, spores were raised to gametophyte stage in petri dishes. Single, genetically unique gametophytes (black) were isolated and used to establish a giant kelp seed bank. No antibiotic treatment was applied, and resident microbes (gray) persisted. In step 5, genetically unique gametophyte germplasm cultures were grown vegetatively in sterile PES. In step 6, M. pyrifera (black) and microbial (gray) DNA of each genetically unique gametophyte culture were co-extracted, followed by shotgun sequencing using an Illumina S4 Novaseq platform. In step 7, microbial DNA was filtered and characterized using the ‘metaxa2’ program with the SILVA128 database. In step 8, microbial networks were constructed and analyzed using the ‘SpiecEasi’ and ‘igraph’ programs.

FIG. 2 is a schematic diagram illustrating that microbial network features of gametophytes vary with sporophyte (adult) biomass. The network features of genetically unique gametophytes (labelled A-C) were analyzed to identify characteristics of early-stage gametophyte microbiomes that influence which individuals will become high biomass sporophytes.

FIG. 3A-FIG. 3C are graphs illustrating relative abundance of bacterial community for four Macrocystis pyrifera populations (AQ=Arroyo Quemado, CP=Camp Pendleton, CI=Channel Islands, LC=Leo Carillo). Taxa were classified and conglomerated to (A) class, (B) order, and (C) family.

FIG. 4A-FIG. 4B are graphs illustrating taxonomic richness and Shannon diversity based on (FIG. 4A) classified species and (FIG. 4B) classified and unclassified species. Taxa were observed in bacterial populations of Macrocystis pyrifera gametophytes from four kelp populations: Arroyo Quemado (AQ), Channel Islands (CI), Camp Pendleton (CP), and Leo Carillo (LC). Pairwise significance was tested with the Kruskal-Wallis test: ns: not significant, *: p<=0.05, **: p<=0.01, ***: p<=0.001, ****: p<=0.0001.

FIG. 5 is a graph illustrating a principal component analysis (PCA) demonstrating compositional difference of microbial communities classified at the species level between gametophyte samples (n=457) with 95% confidence ellipses. Each dot represents a unique female gametophyte from one of four populations: Arroyo Quemado (AQ, medium gray), Channel Islands (CI, medium gray), Camp Pendleton (CP, light gray), and Leo Carillo (LC, dark gray). Distance between points represents how compositionally similar or distinct the microbial communities of gametophytes are.

FIG. 6 is a graph illustrating a principal component analysis (PCA) demonstrating compositional difference of microbial communities classified at the species level between gametophyte samples from bottom and top sporophyte biomass quantiles (<75 g and >235 g, n=230) with 95% confidence ellipses. Each dot represents a unique female gametophyte from one of four populations. Distance between points represents how compositionally similar or distinct the microbial communities of gametophytes are.

FIG. 7A-FIG. 7B are graphs illustrating Venn diagrams of species level core microbes (>0.1% relative abundance and present in at least 75% of samples) shared between all four kelp populations. FIG. 7A illustrates classified species. Six shared species are Flavobacterium sp., Lentilitoribacter donghaensis, Marinobacter sp., Nonlabens ulvanivorans, Ochrobactrum sp. and Sphingopyxis sp. FIG. 7B illustrates classified and unclassified species. Sixteen shared species are Lentilitoribacter donghaensis, Nonlabens ulvanivorans, Ochrobactrum sp., Sphingopyxis sp. and twelve unclassified taxa.

FIG. 8A-FIG. 8D are graphs illustrating taxonomic richness and Shannon diversity for bacterial populations of Macrocystis pyrifera gametophytes at the class level in FIG. 8A, order level in FIG. 8B, family level in FIG. 8C, and genus level in FIG. 8D. Gametophytes are representative of four populations: Arroyo Quemado (AQ), Channel Islands (CI), Camp Pendleton (CP), and Leo Carillo (LC). Pairwise significance was tested with the Kruskal-Wallis test: ns: not significant, *: p<=0.05, **: p<=0.01, ***: p<=0.001, ****: p<=0.0001.

FIG. 9A-FIG. 9D are graphs illustrating taxonomic richness and Shannon diversity for bacterial populations of Macrocystis pyrifera gametophytes at the class level in FIG. 9A, order level in FIG. 9B, family level in FIG. 9C, and genus level in FIG. 9D. Here, all 457 gametophytes were binned into four quantiles according to their wet biomass weight at harvest: Quantile 1 (<75 g), Quantile 2 (75 g to 140.67 g), Quantile 3 (140.67 g to 235 g), and Quantile 4 (>235 g). Pairwise significance was tested with the Kruskal-Wallis test: ns: not significant, *: p<=0.05, **: p<=0.01.

FIG. 10A-FIG. 10D are graphs illustrating principal component analyses (PCA) demonstrating compositional difference of microbial communities between gametophyte samples (n=457) with 95% confidence ellipses at the class level in FIG. 10A, order level in FIG. 10B, family level in FIG. 10C, and genus level in FIG. 10D. Each dot represents a unique female gametophyte from one of four populations: Arroyo Quemado (AQ, medium gray), Channel Islands (CI, medium gray), Camp Pendleton (CP, light gray), and Leo Carillo (LC, dark gray). Distance between points represents how compositionally similar or distinct the microbial communities of gametophytes are.

FIG. 11A-FIG. 11C are graphs illustrating PCA demonstrating compositional difference of microbial communities between gametophyte samples from bottom and top biomass quantiles (<75 g and >235 g, n=230) with 95% confidence ellipses at the order level in FIG. 11A, family level in FIG. 11B, and genus level in FIG. 11C. Each dot represents a unique female gametophyte from one of four populations. Distance between points represents how compositionally similar or distinct the microbial communities of gametophytes are.

FIG. 12A-FIG. 12F are network diagrams illustrating co-occurrence networks of the microbial community sampled from Leo Carillo (LC) gametophytes. Classified at the order level in FIG. 12A and FIG. 12B, the family level in FIG. 12C and FIG. 12D, and the species levels in FIG. 12E and FIG. 12F. Each node represents a unique taxon. Node size represents the hub score and node color represents phylum membership. Edge opacity represents the strength of the link and edge color represents a positive (light gray) or negative (gray) co-occurrence pattern. Microbial networks built from samples of low-biomass gametophytes are shown in FIG. 12A, FIG. 12C, and FIG. 12E (<63.92 g, n=77). Microbial networks built from samples of high biomass gametophytes are shown in FIG. 12B, FIG. 12D, and FIG. 12F (>211 g, n=77).

FIG. 13A-FIG. 13E are graphs illustrating zeta diversity for zeta orders 3, 5, 10, 20, and 50 at the species level, including zeta diversity decline, decline ratio, exponential and power-law regression graphs. Results shown are for zeta orders 3 (FIG. 13A), 5 (FIG. 13B), 10 (FIG. 13C), 20 (FIG. 13D), and 50 (FIG. 13A). Results shown are for gametophytes that became high biomass sporophytes. Columns from left to right: zeta diversity decline representing the number of shared species (Zeta diversity, y-axis) against zeta order; ratio of zeta diversity decline, also called the “retention rate curve” that plots the zeta ratios (Zi+1/Zi) against Zi; zeta decline curves fitted against exponential and power law regressions. AIC scores of the two models confirmed that power law regression is a better fit for all variations.

FIG. 14A-FIG. 14D are graphs illustrating zeta diversity for zeta order 50 at order, family, genus, and species levels, including zeta diversity decline, decline ratio, exponential and power law regression graphs for zeta order 50 at taxonomic levels including order (FIG. 14A), family (FIG. 14B), genus (FIG. 14C), and species (FIG. 14D). Results shown are for gametophytes that became high biomass sporophytes. Columns from left to right: zeta diversity decline representing the number of shared species (Zeta diversity, y-axis) against zeta order; ratio of zeta diversity decline, also called the “retention rate curve” that plots the zeta ratios (Zi+1/Zi) against Zi; zeta decline curves fitted against exponential and power law regressions. AIC scores of the two models confirmed that power law regression is a better fit for all variations.

FIG. 15A-FIG. 15H are graphs illustrating box plots of network topology factors at the order level, including box plots of total nodes (FIG. 15A), total edges (FIG. 15B), positive to negative edge ratio (FIG. 15C), average path length (FIG. 15D), modularity (FIG. 15E), average degree (FIG. 15F), heterogeneity (FIG. 15G), and clustering coefficient for all populations AQ, CI, CP, and LC (FIG. 15H).

FIG. 16A-FIG. 16H are graphs illustrating box plots of network topology factors. Total nodes (FIG. 16A), total edges (FIG. 16B), positive to negative edge ratio (FIG. 16C), average path length (FIG. 16D), modularity (FIG. 16E), average degree (FIG. 16F), heterogeneity(FIG. 16G), and clustering coefficient (FIG. 16H) for all populations (AQ, CI, CP, and LC) with bacteria classified at the family level.

FIG. 17A-FIG. 17D are network diagrams illustrating co-occurrence networks of the microbial community classified at the order level. Each node represents a unique taxon. Node size represents the hub score and node color represents phylum membership. Edge opacity represents the strength of the link and edge color represents a positive (light gray) or negative (gray) co-occurrence pattern. Microbial networks sampled from four populations: AQ in FIG. 17A, CI in FIG. 17B, CP in FIG. 17C, and LC in FIG. 17D.

FIG. 18A-FIG. 18H are graphs illustrating box plots of network topology factors by biomass quantile at the order level. Box plots of total nodes are shown in FIG. 18A, total edges are shown in FIG. 18B, positive to negative edge ratio is shown in FIG. 18C, average path length is shown in FIG. 18D, modularity is shown in FIG. 18E, average degree is shown in FIG. 18F, heterogeneity is shown in FIG. 18G, and clustering coefficient for all biomass quantiles (Q1, Q2, Q3, Q4) with bacteria classified at the order level is shown in FIG. 18H. Pairwise significance was tested with the Wilcoxon test: ns: not significant, *: p<=0.05, **: p<=0.01, ***: p<=0.001, ****: p<=0.0001.

FIG. 19A-FIG. 19H are graphs illustrating box plots of network topology factors by biomass quantile at the family level. Box plots of (FIG. 19A) total nodes, (FIG. 19B) total edges, (FIG. 19C) positive to negative edge ratio, (FIG. 19D) average path length, (FIG. 19E) modularity, (FIG. 19F) average degree, (FIG. 19G) heterogeneity, and (FIG. 19H) clustering coefficient for all biomass quantiles (Q1, Q2, Q3, Q4) with bacteria classified at the family level. Pairwise significance was tested with the Wilcoxon test: ns: not significant, *: p<=0.05, **: p<=0.01, ***: p<=0.001, ****: p<=0.0001.

FIG. 20A-FIG. 20H are graphs illustrating box plots of network topology factors by biomass quantile at the family level. Box plots of (A) total nodes, (B) total edges, (C) positive to negative edge ratio, (D) average path length, (E) modularity, (F) average degree, (G) heterogeneity, and (H) clustering coefficient for all biomass quantiles (Q1, Q2, Q3, Q4) with bacteria classified at the family level. Pairwise significance was tested with the Wilcoxon test: ns: not significant, *: p<=0.05, **: p<=0.01, ***: p<=0.001, ****: p<=0.0001.

FIG. 21A-FIG. 21H. are graphs illustrating box plots of network topology factors by biomass quantile at the genus level. Box plots are shown illustrating (FIG. 21A) total nodes, (FIG. 21B) total edges, (FIG. 21C) positive to negative edge ratio, (FIG. 21D) average path length, (FIG. 21E) modularity, (FIG. 21F) average degree, (FIG. 21G) heterogeneity, and (FIG. 21H) clustering coefficient for all biomass quantiles (Q1, Q2, Q3, Q4) with bacteria classified at the genus level. Pairwise significance was tested with the Wilcoxon test: ns: not significant, *: p<=0.05, **: p<=0.01, ***: p<=0.

FIG. 22A-FIG. 22H are graphs illustrating box plots of network topology factors by biomass quantile at the species level. Box plots of (FIG. 22A) total nodes, (FIG. 22B) total edges, (FIG. 22C) positive to negative edge ratio, (FIG. 22D) average path length, (FIG. 22E) modularity, (FIG. 22F) average degree, (FIG. 22G) heterogeneity, and (FIG. 22H) clustering coefficient for all biomass quantiles (Q1, Q2, Q3, Q4) with bacteria classified at the species level. Pairwise significance was tested with the Wilcoxon test: ns: not significant, *: p<=0.05, **: p<=0.01, ***: p<=0.001, ****: p<=0.0001.

FIG. 23A-FIG. 23B are network diagrams illustrating co-occurrence networks of the microbial community classified at the genus level. Each node represents a unique taxon. Node size represents the hub score and node color represents phylum membership. Edge opacity represents the strength of the link and edge color represents a positive (light gray) or negative (gray) co-occurrence pattern. Microbial networks were made from samples of low-biomass gametophytes (FIG. 23A, <63.92 g, n=77) and high biomass gametophytes (FIG. 23B, >211 g, n=77).

FIG. 24A-FIG. 24D are network diagrams illustrating co-occurrence networks of the microbial community classified at the family level. Each node represents a unique taxon. Node size represents the hub score and node color represents phylum membership. Edge opacity represents the strength of the link and edge color represents a positive (light gray) or negative (gray) co-occurrence pattern. Microbial networks were made from samples of four populations: AQ in FIG. 24A, CI in FIG. 24B, CP in FIG. 24C, and LC in FIG. 24D.

### DETAILED DESCRIPTION OF THE INVENTION

The current global commercial seaweed market is estimated to be nearly $18B and projected to reach nearly $35B by 2030. Most of this market is based in China. With the growing need for sustainable food and biofuel resources, the US has made several investments to become a world leader in seaweed aquaculture. A significant challenge in cultivating seaweed is the development of tools that promote resilient and high-yield crops. The use of growth-promoting microbial inoculants (i.e., the addition of beneficial bacteria) has become a popular practice in agriculture and shown to increase the fitness and yield of several land-based crops such as tomato, wheat, rice, cotton, and maize. Use of these tools has yet to be established in aquaculture but revolutionizes the cultivation of seaweed crops. The present disclosure is the first to analyze seaweed “seed bank” microbiomes in the context of harvest outcomes and create a growth-promoting inoculant. This disclosure covers the invention of a microbial growth-promoting inoculant for increasing the biomass yield of farmed seaweed cultivars. The increased abundance of several species of bacteria (in particular, those from the Mesorhizobium genus) at the early life stage of giant kelp is associated with increased biomass at the adult stage. These bacteria increase the biomass yield of seaweed cultivars. This is useful for commercial applications that benefit from high biomass seaweed feedstocks including, but not limited to, the creation of seaweed-based biofuels. This disclosure also covers the use of microbial community dynamics and network features of early life stage seaweed microbiomes grown in a seed bank setting to increase final biomass yields at the adult stage.

Microbial inoculants can increase the yield of cultivated crops and are successful in independent trials; however, efficacy drops in large-scale applications due to insufficient consideration of microbial community dynamics. The structure of microbiomes, in addition to the impact of individual taxa, is an important factor to consider when designing growth-promoting inoculants. Here, microbial network and community assembly patterns of Macrocystis pyrifera gametophyte germplasm cultures (collectively referred to as a “seedbank”) are used to cultivate an offshore farm in Santa Barbara, California, and identify network features associated with increased biomass of mature sporophytes. This disclosure is based on the finding that: (1) several network features, such as clustering coefficient and edge ratios, significantly vary with biomass outcomes; (2) gametophytes that become low- or high-biomass sporophytes have different hub taxa; and (3) microbial community assembly of gametophyte germplasm cultures is niche-driven. Overall, this study describes microbial community dynamics in M. pyrifera germplasm cultures and ultimately supports the development of early life stage inoculants that can be used on seaweed cultivars to increase biomass yield.

This disclosure also provides development of methods using microbial community dynamics and network features of giant kelp seed bank microbiomes and the distinct characteristics of those associated with early-stage giant kelp cultivars that produce greater biomass yields. These features are used to select high biomass individuals, which improves the efficiency and resource allocation of aquaculture operations.

Given the existing knowledge of microbe-seaweed associations, the present disclosure is applicable to a wide range of aquaculture crops (such as other seaweeds) and agriculture crops as well. This disclosure may be used to predict which individuals become high-biomass individuals, which improves the efficiency and resource allocation of aquaculture operations.

Host-microbe associations are highly specialized to individual hosts and impact plant phenotypes and core functions through direct or indirect mechanisms, such as the supply of nitrogen and disease repression. For this reason, manipulating the microbial composition of plants increases their resilience in challenging environments. Although there is a wealth of research on exploiting host-microbe associations to improve plant fitness, most work has focused on land plants and agricultural crops. The present disclosure presents methods and formulations that produce surprising increases of seaweed biomass in aquaculture.

This invention is not limited to the particular compositions, methods, and experimental conditions described, as such compositions, methods, and conditions may vary. Additionally, the terminology used herein is for the purposes of describing particular embodiments only, and is not intended to be limiting, since the scope of the present invention will be limited only in the appended claims.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The methods and materials are now described herein.

As used in this specification and the appended claims, the singular forms “a”, “an”, and “the” include plural references unless the context clearly dictates otherwise. Thus, for example, references to “the method” includes one or more methods and/or steps of the type described herein which will become apparent to those persons skilled in the art upon reading this disclosure.

The term “agriculturally acceptable excipients”, as used in this specification, refers to compounds that support plant growth, health, and associated microbial communities. Such compounds include pH buffers, seawater, filtered water, purified water, salt, ammonium, minerals such as nitrogen, phosphorus, potassium, strontium, barium, sulfur, cobalt, nickel, chromium, magnesium, iron, copper, manganese, boron, molybdenum, zinc, fluoride, iodine, and nickel, compounds such as monosodium phosphate, ammonium chloride, sodium nitrate, and tris (hydroxymethyl)aminomethane. Other ingredients suitable for growing seaweed are also used.

The term “plant”, as used herein, refers to photosynthetically active organisms with cellulose cell walls that are generally non-motile in the adult diploid form. This includes land and water plants and algae. Plants may have motile spores, gametes, or other reproductive structures, but the largest growth form is generally diploid and non-motile or anchored to a substrate.

The term “seaweed”, as used herein, refers to commercially grown algae and water plants, and includes brown algae or red algae, including kelp, giant kelp, Saccharina latissima, Saccharina japonica, Laminaria digitata, Alaria esculenta, Alaria marginata, Ascophyllum nodosum, Nereocystis luetkeana, Fucus species, Saccharina species, Palmaria palmata, Porphyra species, Eucheuma, Caulerpa lentillifera, Kappaphycus alvarezii, Eucheuma denticulatum, or Undaria species.

The kelp lifecycle is haplodiplontic, which means mature sporophytes release diploid spores that develop into gametophytes that are male and female. The female gametophyte produces specialized haploid egg cells that are fertilized by motile haploid male gametes. The diploid fertilized egg is a zygote that grows into a sporophyte that is still attached to the gametophyte and grows into the large mature kelp that are the main component of ocean kelp forests.

Offshore farms in Southern California provide sufficient light (−25 pmol photons·m⁻²·s^{·1}), nutrients (>1 pM nitrate), and ambient temperature (11-19° C.) for Macrocystis pyrifera to thrive. However, as demonstrated by an offshore test farm in Santa Barbara, seaweed grown in the open ocean has highly variable biomass outcomes (Table 1). Host phenotypes are impacted by several factors including the local environment, host genetics, host gene regulation, and the resident microbiome. Given that this study was performed in the same farm, the effect of environmental variance on M. pyrifera phenotypes in this study is minimized. The genetic factors of M. pyrifera also impact phenotypic outcomes in this farm. Here the impact of the microbiome on host phenotype is described, and formulations of bacteria associated with high biomass individuals are developed for a growth-promoting microbial inoculant to be applied to seaweed crops.

Microbial communities of Macrocystis pyrifera were manipulated through application of gametophyte germplasm cultures from distinct natural populations across Southern California. The germplasm cultures were used to produce sporophytes grown on an offshore farm in Santa Barbara, California. Microbial community differences were compared across populations, and biomass outcomes of mature sporophytes were used to identify taxa that were used in growth-promoting inoculants. The microbial composition of gametophyte germplasm cultures significantly varied across populations, and abundance of unique microbial taxa were significantly associated with high biomass M. pyrifera sporophytes at the gametophyte stage. Microbial taxa associated with increased biomass are used to develop of growth-promoting inoculants. Overall, this work provides a valuable method for use of host-microbe associations in seaweed aquaculture and the development of microbial inoculants that produce high biomass M. pyrifera cultivars to be used as biofuel feedstocks. As the industry moves toward large-scale production, this work complements other tools to improve seaweed farming techniques and to revolutionize the production of seaweed-based biofuels.

Kelp algae includes many species and about 30 genera. Some of them are farmed for commercial use, such as Saccharina latissimi and Saccharina japonica. Kelp is farmed by collecting spores from sporophytes, soaking long strings in media containing the spores and allowing the spores to attach to the strings, growing gametophytes on long strings, anchoring the strings to the ocean floor, then harvesting mature sporophytes. Optimization of varieties for large size and optimizing growth conditions for larger fronds increases the productivity of a farm.

Microbial community dynamics of host-associated microbiomes were analyzed using co-occurrence networks, hub microbes, and community assembly patterns. Co-occurrence networks represent patterns of spatial co-occurrence (i.e., being present together in an environment), and were used to infer relationships between individual taxa. These networks were visually represented as a collection of nodes and edges. In the context of this study, nodes represented unique taxa, and edges represented the links or co-occurrence patterns between unique taxa. Co-occurrence patterns were quantified with measures of network topology such as the clustering coefficient, modularity, and edge ratios. The clustering coefficient and modularity described the division of a network into sub-networks and the density of connections between nodes, respectively. Identification of these sub-communities provided insight on local interactions and their contribution to the overall structure and function of the network. The ratio of positive to negative edges, which represents significant patterns of spatial co-occurrence or exclusion, also indicated the degree to which the community had synergistic or competitive interactions. By investigating how microbial co-occurrence networks at the early life stage of crops varies with crop performance, crop yield is increased, and agricultural inoculants are developed that synergize with network features of high-performing crops.

In one embodiment, the present disclosure relates to a method of obtaining harvested seaweed having at least one desired characteristic including: a) contacting a seaweed spore with a culture media including an inoculum, wherein the inoculum includes an effective amount of at least one microbe; b) growing the spore into a gametophyte; c) outplanting the gametophyte; and d) harvesting the seaweed. In some aspects, the harvested seaweed has at least one desired characteristic that distinguishes the harvested seaweed from non-inoculated seaweed, selected from high biomass, increased growth rate, increased yield, increased stress tolerance, increased resistance to disease, increased pathogen resistance, increased photosynthetic rate, increased tolerance of heavy metals, apical cell growth rate, disease resistance, apical cell multiplication rate, root length, root nodulation, blade length, stalk length, pneumatocyst size, pneumatocyst number, mineral content, chelation of micronutrients, heat tolerance, cold tolerance, or chlorophyll content.

In some aspects, a beneficial property is a desired characteristic. In one aspect, a desired characteristic in seaweed is high biomass. In some other aspects, a desired characteristic in seaweed is increased growth rate. In some other aspects, a desired characteristic in seaweed is increased yield. In some other aspects, a desired characteristic in seaweed is increased nitrogen fixation. In some further aspects, a desired characteristic in seaweed is increased access to nutrients. In some other aspects, a desired characteristic in seaweed is increased secretion of hormones. In still other aspects, a desired characteristic in seaweed is increased tolerance to temperature stress. In other additional aspects, a desired characteristic in seaweed is increased tolerance of heavy metals. In still other aspects, a desired characteristic in seaweed is increased pathogen resistance. In other aspects, a desired characteristic in seaweed is an increased rate of photosynthesis.

In some embodiments, a single microbe is utilized. In some aspects, the single microbe is isolated and purified. In some aspects, the single microbe is a taxonomic species of bacteria. In some aspects, the single microbe is an identifiable strain of a taxonomic species of bacteria. In some aspects, the single microbe is a novel, newly discovered strain of a taxonomic species of bacteria.

In some embodiments, at least one microbe from Table 5 is utilized. In an additional aspect, at least one microbe from Table 6 is utilized. In various aspects, at least one microbe from Table 7 is utilized. In certain aspects, at least one microbe from Table 8 is utilized. In certain aspects, at least one microbe from Table 9 is utilized. In another aspect, at least one microbe from Table 10 is utilized. In a further aspect, at least one microbe from Table 14 is utilized. In one aspect, at least one microbe from Table 15 is utilized.

In some embodiments, at least one bacterium from the order Desulfovibrionales is utilized. In one aspect, at least one bacterium from the order Nitrospinales is utilized. In one aspect, at least one bacterium from the family Magnetococcaceae is utilized. In one aspect, at least one bacterium from the family Beijerinckiaceae is utilized. In one aspect, at least one bacterium from the family Holosporaceae is utilized.

In some embodiments, at least one bacterium from the genus Mesorhizobium is utilized. In one aspect, at least one bacterium from the genus Caldicellulosiruptor is utilized. In an additional aspect, at least one bacteria from the genus Nitratireductor is utilized. In various aspects, at least one bacteria from the genus Aquamicrobium is utilized. In certain aspects, at least one bacterium from the genus Zoogloea is utilized. In a further aspect, at least one bacterium from the genus Aquamicrobium is utilized. In certain aspects, at least one bacterium from the genus Wenyingzhuangia is utilized. In certain aspects, at least one bacterium from the genus Pedobacter is utilized. In certain aspects, at least one bacterium from the genus Pedobacter is utilized.

In some embodiments, at least one bacterium from the species Zoogloea ramigera is utilized, either individually or in combination with one or more additional bacterial species, or any variants thereof. In one aspect, at least one bacterium from the species Zoogloea ramigera is utilized as part of a microbial consortium. In an additional aspect, at least one bacterium from the species Mesorhizobium genosp. is utilized as part of a microbial consortium. In various aspects, at least one bacterium from the species Zoogloea ramigera is utilized as part of a microbial consortium. In certain aspects, at least one bacterium from the species Marinomonas brasilensis is utilized as part of a microbial consortium. In a further aspect, at least one bacterium from the species mucus bacterium 80 is utilized as part of a microbial consortium. In another aspect, at least one bacteria from the species Zoogloea ramigera is utilized as part of a microbial consortium.

In some aspects, the single microbe-whether a taxonomically identifiable species or strain—is combined with one or more other microbes of a different species or strain. In certain aspects, the combination of two or more microbes forms a consortia or consortium. The terms consortia and consortium are utilized interchangeably.

In one aspect, the at least one desired characteristic is high biomass. In another aspect, the non-inoculated seaweed is selected from a parent sporophyte, a sporophyte of the same variant, a sporophyte of the same gametophyte culture, a sporophyte of the same cultivar, or a sporophyte of the same genotype. In some aspects, the inoculum includes an effective amount of at least one microbe isolated from a seaweed, one or more spores from a seaweed, or one or more seaweed gametophytes, or any combination thereof. In one aspect, the inoculum includes an effective amount of at least one microbe isolated from a high biomass seaweed, one or more spores from a high biomass seaweed, or one or more gametophytes from a high biomass seaweed, or any combination thereof. In one aspect, the at least one microbe is selected from Marinomonas brasilensis, mucus bacterium 80, Zoogloea ramigera, Mesorhizobium species, Nitratireductor species, Aquamicrobium species, Pedobacter species, Wenyinzhuangia species, species from the order Desulfovibrionales, species from the order Nitrospinales, species from the family Magnetococcaceae, species from the family Beijerinckiaceae, species from the family Holosporaceae, species from the family Brucellaceae, species from the order Sphingomondales, and species from the order Thermoanaerobacterales; or any combination thereof. In one aspect, the inoculum includes at least one microbe selected from Mesorhizobium species, Wenyingzhuangia species and Pedobacter species; or a variant thereof. In another aspect, the inoculum includes at least one Mesorhizobium species or a variant thereof. In various aspects, the inoculum optionally does not contain species from the genus Labrenzia. In various aspects, the inoculum optionally does not contain species from the genus Aquamarina. In one aspect, the inoculum optionally does not contain species from the genus Sneathiella. In certain aspects, the inoculum optionally does not contain species from the genus Pseudohaliea. In a further aspect, the inoculum optionally does not contain species from the genus Saccharospirillum. In certain aspects, the culture media includes agriculturally acceptable excipients. In one aspect, the techniques described herein relate to a method, wherein the seaweed is Macrocystis pyrifera or a variant thereof. In one aspect, the seaweed spore in b) is a Macrocystis pyrifera spore or a variant thereof. In an additional aspect, the culture media includes sterile Provasoli enriched seawater medium.

In one embodiment, the disclosure provides an agricultural formulation for an inoculant including a mixture of two or more bacterial species selected from Marinomonas brasilensis, mucus bacterium 80, Zoogloea ramigera, Mesorhizobium species, Nitratireductor species, Aquamicrobium species, Pedobacter species, Wenyinzhuangia species, species from the order Desulfovibrionales, species from the order Nitrospinales, species from the family Magnetococcaceae, species from the family Beijerinckiaceae, species from the family Holosporaceae, species from the family Brucellaceae, species from the order Sphingomondales, or species from the order Thermoanaerobacterales. In one aspect, the agricultural formulation further includes at least one agriculturally acceptable excipient. In an additional aspect, the agricultural formulation does not contain species from the genus Labrenzia. In one aspect, the inoculum does not contain species from the genus Aquamarina. In an additional aspect, the inoculum does not contain species from the genus Sneathiella. In various aspects, the inoculum does not contain species from the genus Pseudohaliea. In certain aspects, the inoculum does not contain species from the genus Saccharospirillum. In a further aspect, the inoculum optionally does contain species from the genus Aquamarina. In another aspect, the inoculum optionally does contain species from the genus Sneathiella. In one aspect, the inoculum optionally does contain species from the genus Pseudohaliea. In an additional aspect, the inoculum optionally does contain species from the genus Saccharospirillum. In various aspects, the agricultural formulation optionally contains species from the genus Labrenzia.

In one embodiment, the disclosure provides a method of making an inoculant including: a) collecting a sporophyte from a defined geographical region; b) collecting a spore from the sporophyte; c) analyzing at least one microbe obtained from the sporophyte; d) growing a mature sporophyte from the spore; e) identifying the mature sporophyte as a high biomass sporophyte; f) culturing a microbial community of the high biomass sporophyte in biomass media; and g) adding at least one agriculturally acceptable excipient to the biomass media to make an inoculant. In one aspect, the high biomass sporophyte is a Macrocystis pyrifera sporophyte.

In one embodiment, the disclosure provides a method of obtaining a high biomass seaweed including incubating at least one spore or gametophyte with a culture media including the inoculant described herein.

In one embodiment, the present disclosure provides a method of obtaining high biomass seaweed including: a) releasing a spore from a sporophyte in a culture media; b) identifying at least one bacterial species present in the culture media; c) determining that the at least one bacterial species is associated with a high biomass seaweed; d) growing the spore into a gametophyte; e) outplanting the gametophyte; and f) harvesting a high biomass seaweed. In one aspect, the high biomass seaweed is Macrocystis pyrifera. In certain aspects, the culture media is sterile Provasoli enriched seawater medium. In some aspects, determining that the at least one bacterial species is associated with a high biomass seaweed includes modeling network features of a microbiome of the spore to determine that the bacterial species is associated with a high biomass seaweed. In various aspects, the network features used are chosen from clustering coefficients, positive to negative edge ratios, modularity, heterogeneity or average path length.

Hub microbes are central to the process of microbiome recruitment and have several associations across the microbial network. They are identified with network topology data and defined by having a disproportionate number of links with other taxa in the network. Hub microbes are drivers of the overall microbial community because of their ability to recruit and support the introduction of other bacteria that directly benefit the host, particularly at the early life stage of crops. The impact of hub microbes on the diversity of host microbiomes occurs directly; for example, by impacting the colonization of other microbes, or indirectly; for example, through the host. While hub microbes are not typically the focus of agricultural inoculants, they are used in tandem with growth-promoting bacteria to improve crop fitness by increasing native recruitment of beneficial bacteria and supporting synergistic interactions. Furthermore, the use of inoculants that do not compete with hub taxa also improves long-term success and facilitates beneficial changes in the overall community.

In certain aspects, the disclosure provides for the development of highly functional microbial consortia that promote the development and expression of a desired phenotype or genotype in a seaweed species. In some embodiments, the consortia of the present disclosure possess functional attributes that are not found in nature, when the individual microbes are living alone. That is, in various embodiments, the combination of particular microbial species into consortia, leads to the microbial combination possessing functional attributes that are not possessed by any one individual member of the consortia when considered alone.

In some embodiments, this functional property possessed by the microbial consortia is the ability to impart one or more beneficial properties to a seaweed species, including but not limited to: high biomass, increased growth rate, increased yield, increased nitrogen fixation, increased pathogen resistance, increased resistance to disease, increased tolerance to temperature stress, increased tolerance of heavy metals, and increased photosynthetic rate.

However, in other embodiments, the disclosure provides for individual isolated and biologically pure microbes that impart beneficial properties upon a desired seaweed species, without the need to combine said microbes into consortia.

In various aspects, the microbial consortia is any combination of individual microbes from Table 5. In one aspect, the microbial consortia is any combination of individual microbes from Table 6. In yet an additional aspect, the microbial consortia is any combination of individual microbes from Table 7. In certain aspects, the microbial consortia is any combination of individual microbes from Table 8. In a further aspect, the microbial consortia is any combination of individual microbes from Table 9. In another aspect, the microbial consortia is any combination of individual microbes from Table 10. In one aspect, the microbial consortia is any combination of individual microbes from Table 14. In an additional aspect, the microbial consortia is any combination of individual microbes from Table 15. In various aspects, the microbial consortia is any combination of individual microbes from any of Tables 5-10 and 14-15. In a further aspect, the microbial consortia comprise two microbes, or three microbes, or four microbes, or five microbes, or six microbes, or seven microbes, or eight microbes, or nine microbes, or 10 microbes, or more than 10 microbes.

Another object of the disclosure relates to the use of the isolated microbes and microbial consortia as seaweed growth promoters. In other aspects, the isolated microbes and microbial consortia function as seaweed growth modifiers, which can, e.g. subvert normal senescence and lead to increased biomass.

Yet another object of the disclosure relates to the use of the isolated microbes and microbial consortia as seaweed health enhancers.

Another object of the disclosure is to design a microbial consortium, which is able to perform multidimensional activities in common. In certain aspects, the microbes comprising the consortium act synergistically. In aspects, the effect that the microbial consortium has on a certain seaweed characteristic is greater than the effect that would be observed had any one individual microbial member of the consortium been utilized singularly. That is, in some aspects, the consortium exhibits a greater than additive effect upon a beneficial property, as compared to the effect that would be found if any individual member of the consortium had been utilized by itself.

In some aspects, the consortia lead to the establishment of other seaweed-microbe interactions, e.g. by acting as primary colonizers or founding populations that set the trajectory for future microbiome development. Microbial communities assemble in a niche-driven manner across all biomass outcomes. When designing inoculants to increase the biomass yield of M. pyrifera cultivars, avoiding competition with hub taxa identified here increases desired traits. Introduction of desired hub taxa at the gametophyte stage induces the recruitment of other beneficial bacteria and shapes the overall community.

In some aspects, the individual microbes of the disclosure, or consortia comprising same, are combined into an agriculturally acceptable composition.

In some embodiments, the agricultural compositions of the present disclosure include, but are not limited to: antibiotics, sterilants, phosphorus, nitrogenous salts, ammonia, potassium, micronutrients, cobalt, magnesium, pH buffers, amino acids, yeast extract, tryptone, beef extract, peptone, potassium nitrate, ammonium nitrate, ammonium chloride, ammonium sulfate, ammonium phosphate, ammonia or combinations thereof. Inorganic salts, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate can be used alone or in combination.

In one embodiment of the present disclosure, the microbes (including isolated single species, or strains, or consortia), are supplied in the form of spore coatings or other applications to the spore. In one aspect, the spore coating is applied to a naked and untreated spore. In still another aspect, the spore coating is applied as an overcoat to a previously treated spore. In another aspect of the disclosure, the microbes (including isolated single species, or strains, or consortia) are supplied in the form of saprophyte coatings or other applications to the saprophyte. In another aspect, the saprophyte coating is applied to a naked and untreated saprophyte. In still another aspect, the saprophyte coating v applied as an overcoat to a previously treated saprophyte. In another aspect of the disclosure, the microbes (including isolated single species, or strains, or consortia) are supplied in the form of gametophyte coatings or other applications to the gametophyte. In another aspect, the gametophyte coating is applied to a naked and untreated gametophyte. In still another aspect, the gametophyte coating is applied as an overcoat to a previously treated gametophyte.

In embodiments, the agricultural compositions of the disclosure are formulated as liquid solutions, wettable powders, dusting powders, soluble powders, emulsions or suspension concentrates, dressings, tablets, water-dispersible granules, water soluble granules (slow or fast release), microencapsulated granules or suspensions, and as irrigation components, among others. In certain aspects, the compositions are diluted in an aqueous medium prior to application.

Still another object of the disclosure relates to the agricultural compositions being formulated to provide a high colony forming units (CFU) bacterial population or consortia. In one aspect, the agricultural compositions have adjuvants that provide for a pertinent shelf life. In another aspect, the CFU concentration of the taught agricultural compositions is higher than the concentration at which the microbes would exist naturally, outside of the disclosed methods. In another aspect, the agricultural composition contains the microbial cells in a concentration of 103-1012 CFU per gram of the carrier or 105-109 CFU per gram of the carrier. In an aspect, the microbial cells are applied as a coat directly to a spore at a concentration of 105-109 CFU. In other aspects, the microbial cells are applied as an overcoat on top of another spore coat at a concentration of 105-109 CFU.

In one aspect, the disclosure is directed to agricultural microbial formulations that promote seaweed growth. In an additional aspect, the disclosure provides for the taught isolated microbes, and consortia comprising same, to be formulated as an agricultural inoculant.

The disclosed polymicrobial formulations can: lower the need for nitrogen containing fertilizers, improve mineral solubilization, protect against pathogens, and make valuable nutrients available to the seaweed, such as phosphate, thus reducing and eliminating the need for using chemical pesticides and chemical fertilizers.

In one embodiment, the isolated and biologically pure microbes of the present disclosure are utilized in a method of imparting one or more beneficial properties or traits to a desired seaweed species, or a variant or homolog thereof.

In one embodiment, the agriculturally acceptable composition containing isolated and biologically pure microbes of the present disclosure are utilized in a method of imparting one or more beneficial properties or traits to a desired seaweed species.

In one embodiment, the disclosure provides a method of obtaining a high biomass seaweed, including: a) contacting a seaweed spore with a culture media including an inoculum; b) growing the seaweed spore into a gametophyte; c) outplanting the gametophyte; and d) harvesting the high biomass seaweed. In one aspect of the methods described herein, the inoculum includes a plurality of microbes isolated from high biomass seaweed. In another aspect of the methods described herein, the inoculum includes bacteria selected from Marinomonas brasilensis, mucus bacterium 80, Zoogloea ramigera, Mesorhizobium species, Nitratireductor species, Aquamicrobium species, Pedobacter species, Wenyinzhuangia species, species from the order Desulfovibrionales, species from the order Nitrospinales, species from the family Magnetococcaceae, species from the family Beijerinckiaceae, species from the family Holosporaceae, species from the family Brucellaceae, species from the order Sphingomondales, and species from the order Thermoanaerobacterales. In one aspect, the inoculum includes bacteria selected from a group consisting of Mesorhizobium species, Wenyingzhuangia species and Pedobacter species. In some aspects, the inoculum includes Mesorhizobium species. In another aspect, the method includes culture media that includes agriculturally acceptable excipients. In a further aspect, the inoculum does not contain species from the genus Labrenzia. In another aspect, the inoculum optionally contains species from the genus Labrenzia. In related aspects, the method relates to a high biomass seaweed, wherein the high biomass seaweed is Macrocystis pyrifera. In another aspect, the method provided herein includes a culture media, wherein the culture media includes sterile Provasoli enriched seawater medium.

In one aspect, the disclosure is directed to an agricultural formulation for a high biomass seaweed inoculant including a mixture of two or more bacterial species selected from Marinomonas brasilensis, mucus bacterium 80, Zoogloea ramigera, Mesorhizobium species, Nitratireductor species, Aquamicrobium species, Pedobacter species, Wenyinzhuangia species, species from the order Desulfovibrionales, species from the order Nitrospinales, species from the family Magnetococcaceae, species from the family Beijerinckiaceae, species from the family Holosporaceae, species from the family Brucellaceae, species from the order Sphingomondales, and species from the order Thermoanaerobacterales. In one aspect, the agricultural formulation also includes at least one agriculturally acceptable excipient. In one aspect, the formulation does not contain species from the genus Labrenzia. In one aspect, the formulation does not contain a species from the Class Cytophagia. In another aspect, the formulation optionally contains a species from the Class Cytophagia.

In one aspect, the disclosure describes a method of making an inoculant including: a) collecting a sporophyte from a defined geographical region; b) collecting a spore from the sporophyte; c) analyzing a plurality of microbes from the sporophyte; d) growing a mature sporophyte from the spore; e) identifying the mature sporophyte as a high biomass sporophyte; f) culturing a microbial community of the high biomass sporophyte in biomass media; and g) adding at least one agriculturally acceptable excipient to the biomass media to make an inoculant. In one aspect of the method described herein, the high biomass sporophyte is Macrocystis pyrifera. In one aspect, the method of making an inoculant described herein describes a high biomass sporophyte, wherein the high biomass sporophyte is Macrocystis pyrifera. In another aspect, the method described herein describes a formulation including culture media, wherein the culture media is sterile Provasoli enriched seawater medium.

In yet another aspect, the disclosure provides a method of obtaining high biomass seaweed including: a) releasing a spore from a sporophyte in a culture media; b) identifying a bacteria species present in the culture media; c) determining that the bacteria species is associated with a high biomass seaweed; d) growing the spore into a gametophyte; e) outplanting the gametophyte; and f) harvesting a high biomass seaweed. In one aspect, the high biomass seaweed is Macrocystis pyrifera. In another aspect, the method may include culture media, and in some aspects, the culture media is sterile Provasoli enriched seawater medium. In a further aspect, the method includes modeling network features of the microbiome to determine that the bacteria species is associated with a high biomass seaweed. In an additional aspect, the network features used are chosen from clustering coefficients, positive to negative edge ratios, modularity, heterogeneity and average path length.

While co-occurrence network and hub microbe analyses, as described above, are used to display representative microbiomes for a group of hosts, community assembly patterns drive mechanisms controlling the variability of microbiomes across hosts. Two common forms of community assembly follow a stochastic or niche assembly process. During stochastic assembly, microbes are randomly incorporated from the environment into a community. During niche assembly, the likelihood of species being incorporated is dependent on their ecological role and those of existing community members. The relative likelihood of these two assembly processes is displayed using the zeta diversity framework, a method for calculating the number of shared species across a large number of sample sites. As the number of sites being compared increases, zeta diversity typically decays following an exponential or power-law form. An exponential decay shows that communities are more likely to be assembled stochastically, while a power-law decay shows they are more likely to be assembled via niche differentiation. In the context of this study, manipulation of microbial communities to assemble in a stochastic or niche-driven manner improves inoculant design. If the assembly is niche-driven, for example, inoculants are designed to avoid competition with established niches and to increase establishment rates.

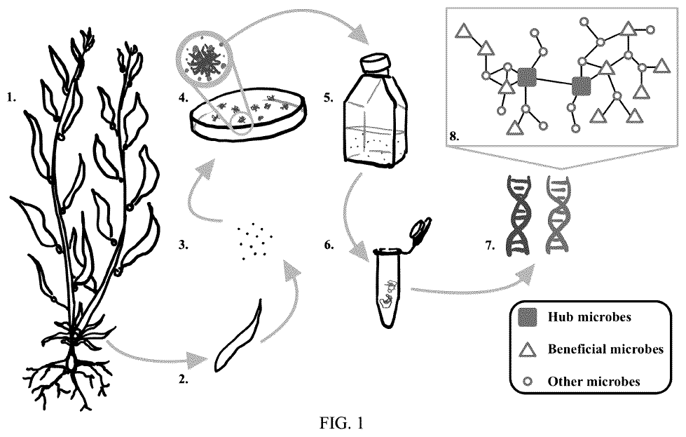

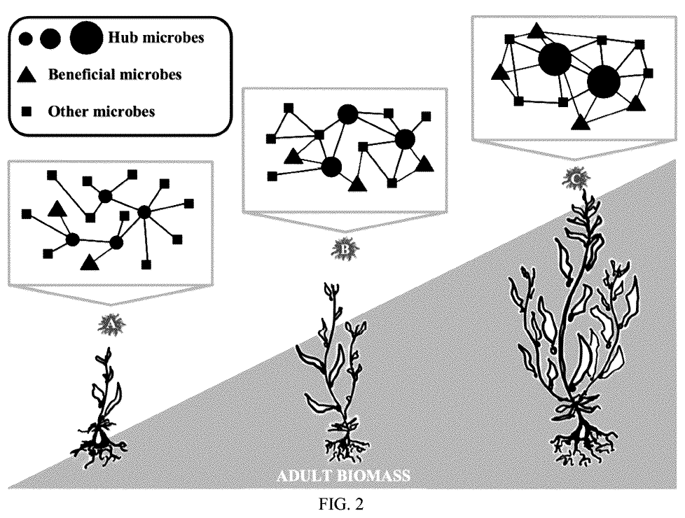

Analysis of microbial community dynamics using the methods described above allows for a more precise development of microbial inoculants that increase crop yield. The present disclosure provides methods and formulations including Macrocystis pyrifera (giant kelp) gametophyte germplasm cultures, collectively referred to as a “seed bank,” that were used to cultivate an offshore farm in Santa Barbara, California (FIG. 1). There is a significant difference in microbial community composition between gametophytes that become high- versus low-biomass sporophytes, and bacteria within the Mesorhizobium genus are key components for creating a growth-promoting inoculant. The present disclosure includes use of the topology of microbiome co-occurrence networks, the relative likelihoods of two common community assembly processes for giant kelp seed bank cultures, and the relationships of these network features with the final biomass yield of mature sporophytes to develop inoculants to support growth of high biomass sporophytes. Given that microbe-microbe interactions impact overall microbiome function and host condition, the final yield of M. pyrifera adult sporophytes is in part influenced by differences in microbial community dynamics during the gametophyte stage. The microbiomes of gametophytes that become high biomass sporophytes have co-occurrence patterns that support synergistic interactions and have hub microbes that are distinct from those of gametophytes that become low-biomass sporophytes. Given the tight ecological interactions between microbes and their seaweed hosts, seed bank microbial communities assemble through niche differentiation. Microbial network characteristics of early-stage gametophytes affect which gametophytes become high biomass sporophytes (FIG. 2). Following successful introduction of growth-promoting inoculants during the gametophyte stage, biomass of resulting sporophytes is increased. Overall, this disclosure provides valuable methods and formulations for microbial inoculants that are used in the development of gametophyte germplasm banks for the production of high biomass seaweed cultivars. High biomass cultivars are used, for example, as biofuel feedstocks.

### EXAMPLES

### Example 1

### Sporophyte Collection.

Sporophylls, the reproductive blades of Macrocystis pyrifera that contain spores, were collected from natural kelp beds in the fall of 2018 (early December) from four Southern California regions: (1) the Santa Barbara Channel (Arroyo Quemado, n=60), (2) the Channel Islands (Catalina Island at the Wrigley Marine Science Center, n=60), (3) the South (Camp Pendleton, n=60), and (4) a hybrid zone (Leo Carrillo, n=370; Table 2). These locations represent four distinct populations that correspond to areas of genetic divergence in the region and were sampled to start a germplasm collection of gametophytes. Sporophylls were shipped overnight to the University of Wisconsin-Milwaukee for gametophyte isolation and DNA extraction.

### Gametophyte Isolation and DNA Extraction.

One day after sampling, each sporophyll was cleaned and spore release was induced following the Oppliger method. Briefly, spores were released in sterile Provasoli enriched seawater medium (PES) made with Instant Ocean Sea Salt and ultrapure water (Symplicity water system) to a salinity of 34. Spores were then inoculated in 60 mm×15 mm Petri dishes with 10 mL of PES. For each sporophyte donor, two dilutions were used to obtain final densities of 10 spores-mm⁻² and 100 spores·mm⁻² that allowed for single gametophyte isolation. Petri dishes were placed in a plant growth chamber under red light (fluorescent tubes wrapped with red cellophane paper) with a light intensity of 20 pmol photons·m⁻²·s⁻¹ (checked with Quantum/Radiometer /Photometer LI-COR 185A) and a 12:12 h (Light:Dark) photoperiod at 12° C. PES medium was replaced every two weeks while the spores were in this low density phase. Once gametophytes had grown to approximately 100 pm, a Pasteur pipette that had the tip thinned out by a Bunsen burner was used under an inverted microscope to isolate and place single gametophytes in a 24-well plate. Gametophyte sex was identified based on cell size. Dimorphism between female and male gametophytes is characteristic in Laminareales; in Macrocystis pyrifera, female gametophyte cells are 5 to 7 times larger than male cells.

All gametophyte cultures were grown under red light (30 pmol photons·m⁻²·s^{·1}), at 12° C. temperature. Higher light intensity at this stage was used to induce faster vegetative growth and to bulk up the biomass of each gametophyte culture, since gametophytes had grown larger and adapted to increased light. PES medium was replaced every week while cultures were in the 24-well plates. After two months of growth, the isolated gametophytes were approximately 2 mm in diameter. At this stage, to promote exponential growth of clone biomass, selected clones were fragmented mechanically by repeated pipetting in the plate well. After an additional month of growth, selected clones were transferred into 75 mL cell culture horizontal bottles. PES medium was replaced every week and periodically fragmented using a portable mini drink mixer inside the culture bottle. Prior to DNA extraction, aliquots of each gametophyte were gently centrifuged to the bottom of an Eppendorf tube and the supernatant medium was discarded to obtain 50 to 100 mg of gametophyte biomass. The gametophyte tissue was then pulverized using liquid nitrogen. High quality genomic DNA from a total of 500 female and 100 male gametophyte lines was extracted using the NucleoSpin 96 Plant Kit (Macherey-Nagel, Duren, Germany). There was no antibiotic treatment prior to DNA extraction; therefore, the haploid DNA of each giant kelp gametophyte culture was co-extracted with its associated microbiota.

### Shotgun Sequencing of Gametophytes and Quality Filtering of Reads.

Extracted DNA was sent to the BGI North America NGS lab for library prep and whole-genome re-sequencing. After library prep, samples were sequenced using an Illumina S4 Novaseq platform, generating 11.2 GB of 150 base pair reads per sample. 562 samples were sequenced in total. Raw fastq files were processed using the fastp program, which filters out low quality reads, cuts bases with low quality, and trims off adapter sequences. Due to the lack of a published Macrocystis pyrifera genome at the time of analysis, and evidence of bacterial contamination in previously published brown macroalgae genomes, all reads were included in the classification pipeline (see “Classification and normalization of quality filtered and trimmed reads”) to ensure that all candidate bacterial reads were analyzed.

### Sporophyte Production, Farm Design, and Phenotyping.

A total of 2,500 sporophytes (500 unique genotypes with five replicates each) were grown in an offshore farm one mile off the coast of Santa Barbara, California. Sporophytes were produced from the cross of 500 unique female gametophytes (370 from Leo Carillo, and 60 each from Arroyo Quemado, Catalina Island, and Camp Pendleton) with a single male from Leo Carillo. Gametophyte cell cultures were fragmented into filaments approximately 5-10 cells long by placing culture samples in 1.5 mL Eppendorf tubes and gently rotating with a pellet pestle. Female gametophyte fragments were then seeded on polyvinyl lines (6 cm long, 2 mm diameter), followed by male fragments the next day. The lines were then exposed to white light, which was increased gradually from 15, 22, 35, to 60 pmol photons·m⁻²·s⁻¹ on the first, second, third, and fourth days following inoculation, respectively. Crosses grew for one month until sporophytes developed, and were shipped in acrylic vials with culture medium overnight to a marine laboratory at the University of California, Santa Barbara (UCSB).

Upon arrival to UCSB, the vials were placed in tanks with running seawater to maintain temperature and white light exposure at 100 pmol photons·m⁻²·s⁻¹. To facilitate out-planting to the offshore farm long-lines, the sporophyte strings were attached to a ¼-inch nylon seeding line at 0.5 m intervals in the laboratory the day before out-planting. This was done by inserting the sporophyte strings through the weave of the seeding line. Ten 25 m long seedling lines, each containing 50 sporophyte strings (described above) were fastened in series by divers to the backbone of 1-inch diameter long-lines, resulting in 250 genotypes per longline. One replicate set of the 500 genotypes was out-planted to two adjacent longlines on the farm on the first week of May 2019. The position of the ten 25 m seeding lines that made up one replicate of the 500 genotypes was stratified along the five pairs of long-lines to minimize the potential for positioning on the long-line to effect phenotypic expression.

All out-planted kelp was harvested between Sep. 7-12, 2019, using Santa Barbara Mariculture's vessel Perseverance. A total of 1,677 plants were harvested, representing an outplant survivorship of 67% for the four-month grow-out period. All plants were returned to the UCSB marine laboratory in mesh bags on the day they were harvested and stored overnight in shaded tanks equipped with filtered flow-through seawater. The following morning, plants were damp-dried by spinning for 20 seconds, then weighed to obtain total biomass. The total biomass for individual replicates was averaged to obtain a single value for each unique genotype (n=500). Of the 562 gametophyte samples sequenced, 457 samples had corresponding phenotype data and were used in analysis for this study. The other 105 samples did not have phenotype data because of premature loss during or death before harvest. For some of the analyses described below, unique genotypes were either binned by population (Arroyo Quemado, Catalina Island, Camp Pendleton, and Leo Carillo) or into biomass quantiles for the entire dataset and each population individually (Table 1).

### Classification and Normalization of Quality Filtered and Trimmed Reads.

Partial 16S rRNA sequences were extracted from raw shotgun sequence reads and classified to the finest possible resolution using the metaxa2 package (version 2.2.2). Classified reads were then conglomerated to broader taxonomic ranks using the phyloseq package in R (RStudio 2019, R 2020). The BLAST+ search option and SSU_SILVA128 reference database were used. The metaxa2 traversal tool and data collector were used to organize the identified SSU sequences into a taxonomy table and a raw abundance table, which were further analyzed and processed with the phyloseq package in R. In alignment with recommended treatment of sequenced datasets, a compositional approach was used, and read counts were not rarified. Raw abundance counts were processed by removing singletons and doubletons, normalizing counts by sequencer, averaging counts for samples that were sequenced over multiple runs, and again removing any remaining singletons and doubletons. Only taxa from the bacterial domain were kept for analysis.

Unless stated otherwise, for all steps described below, analyses were performed with bacterial classifications conglomerated to five levels (class, order, family, genus, and species) for all gametophyte samples (n=457). Analyses at the class, order, family, genus, and species levels were performed to address the challenge of taxonomic resolution and classification uncertainty at higher levels (i.e., genus and species) and to consider lower levels (i.e., class, order, and family) as proxies for ecological function. For kelp population comparisons, gametophytes were grouped according to the geographic region of their parent sporophyte: Arroyo Quemado (n=52), Catalina Islands (n=44), Camp Pendleton (n=53), and Leo Carillo (n=308). As described above, analyses were also performed with gametophytes grouped by quantile of adult sporophytes' biomass instead of by population (Table 1).

Classification of Bacterial Community Reveals Evidence of a Core Microbiome Across all Four Populations of Macrocystis pyrifera Gametophytes.

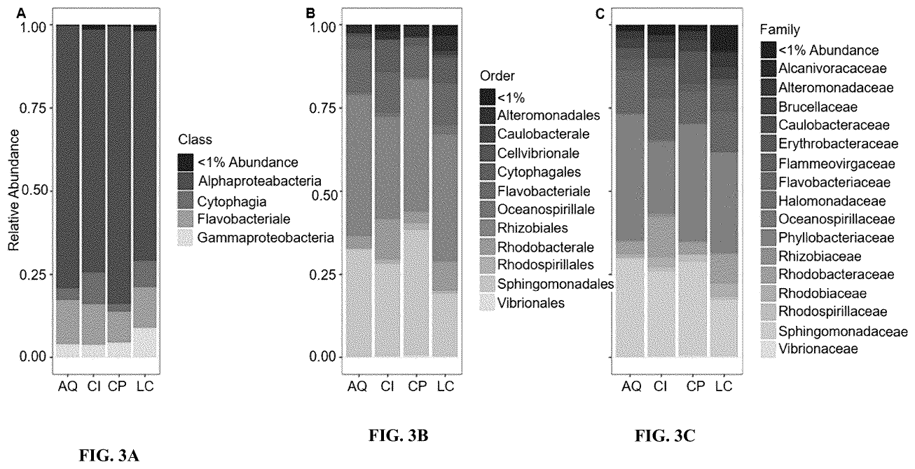

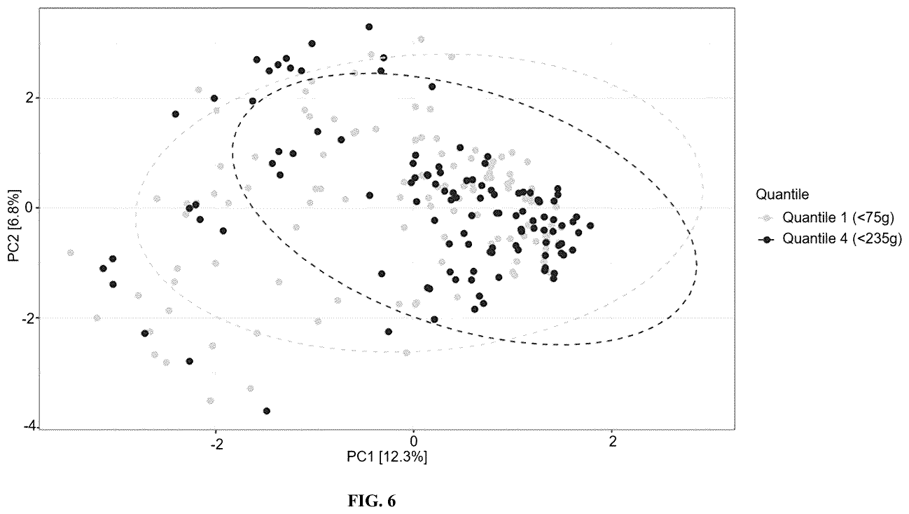

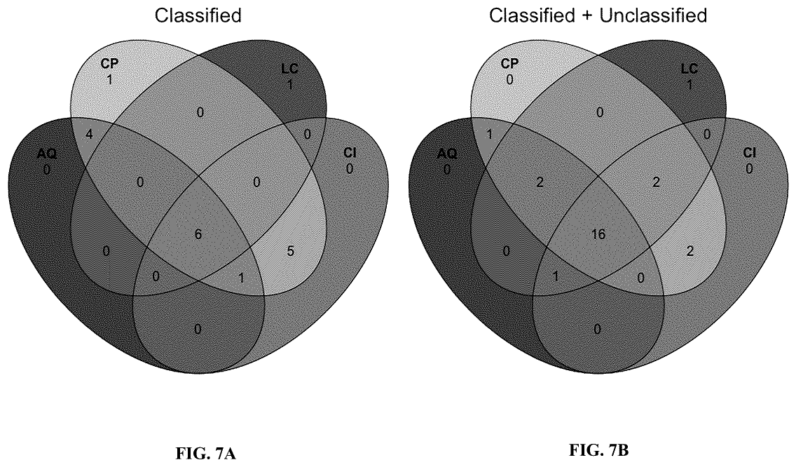

Shotgun sequence data from a total of 457 Macrocystis pyrifera gametophytes was used to characterize the microbial community across all samples. A total of 1,594 species-level taxa were identified across the collective set of M. pyrifera gametophytes, of which 1,106 species-level taxa were classifiable with the SILVA 128 database (Table 3 and Table 4). Taxa with >1% relative abundance were recorded in each individual population, and across all populations (Table 9). Across all populations, the most abundant taxa belonged to the classes Alphaproteobacteria, Flavobacteriia, Cytophagia, and Gammaproteobacteria (FIG. 3A, Table 5). Highly abundant orders included Rhizobiales, Sphingomonadales, Flavobacteriales, Cytophagales, Rhodobacterales, and Alteromonadales (FIG. 3B, Table 5). Highly abundant families included Phyllobacteriaceae, Sphingomonadaceae, Flavobacteriaceae, Flammeovirgaceae, Rhodobacteraceae, and Alteromonadaceae (FIG. 3C, Table 5). At the species level, highly abundant taxa included Lentilitoribacter donghaensis, Sphingopyxis sp., Nonlabens ulvanivorans, Marinobacter sp., and Ochrobactrum sp. (Table 5). Analysis of species-level taxa with >0.1% relative abundance and present in at least 75% of samples revealed that Flavobacterium sp., Lentilitoribacter donghaensis, Marinobacter sp., Nonlabens ulvanivorans, Ochrobactrum sp. and Sphingopyxis sp. constitute a core microbiome shared across all populations (FIG. 7A-FIG. 7B).

### Fraction Unclassified and Taxonomic Richness (Alpha Diversity).

Metaxa2 classifies reads to the highest resolution. Some reads are identified as “unclassified” at various taxonomic levels. To calculate the fraction of unclassified taxa, absolute abundance counts of bacteria were first conglomerated to the class, order, family, genus, or species level. Taxa that were not present for subsets of the gametophyte data (described above) were removed. Taxa that included “unclassified” in the name were also removed and counted. The number of unclassified taxa was then divided by the sum of unclassified taxa plus the remaining taxa in the taxonomy table (Table 3 and Table 4). Observed taxonomic richness and Shannon diversity for classified species and all species including unclassified species was calculated using the estimate_richness command from the phyloseq package. In the present disclosure, taxonomic richness is defined as the number of unique taxa observed in an individual sample. Shannon diversity takes into account taxonomic richness and evenness. Reported measures represent the average alpha diversity metric (taxonomic richness or Shannon diversity) per individual. The significance with which the alpha diversity of microbial communities differed between groups was evaluated using a Kruskal-Wallis test (p=0.05), which accounts for unequal sample sizes.

### Alpha Diversity Varies by Population.

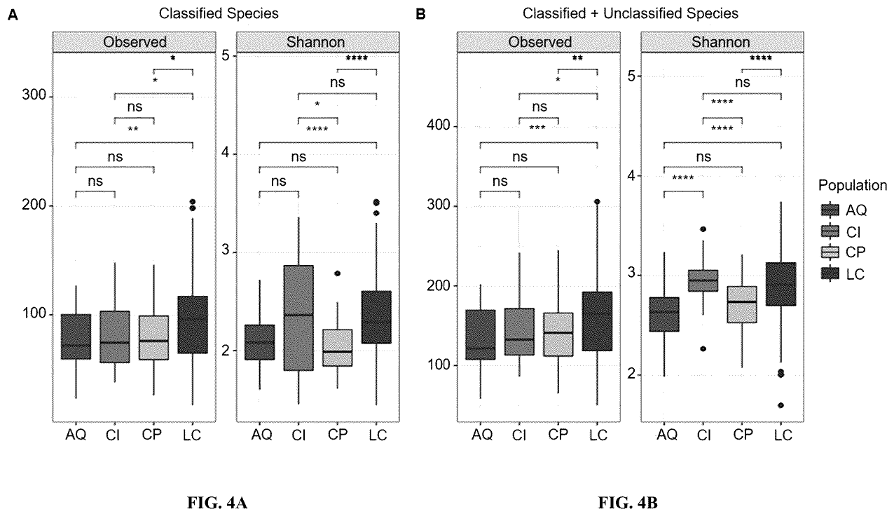

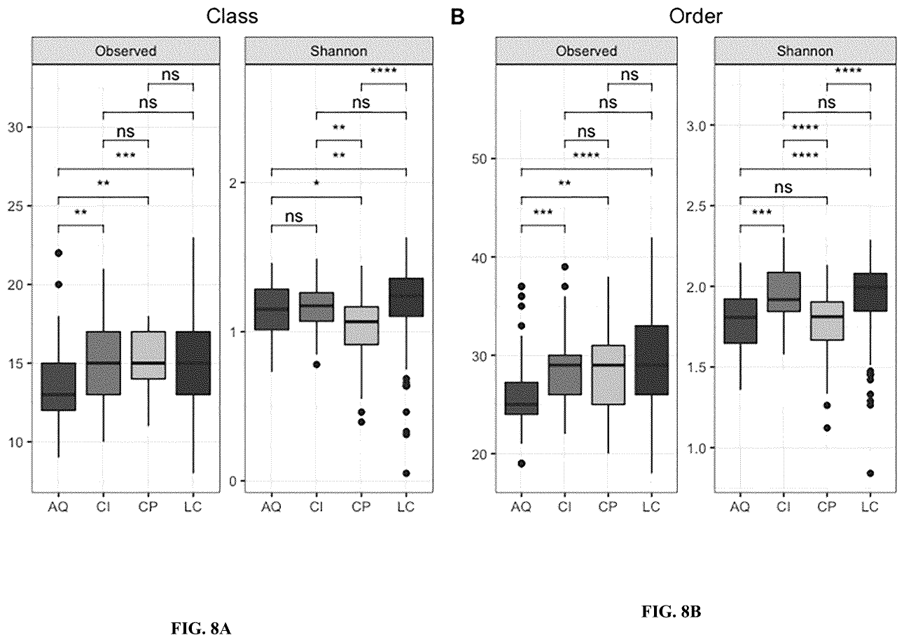

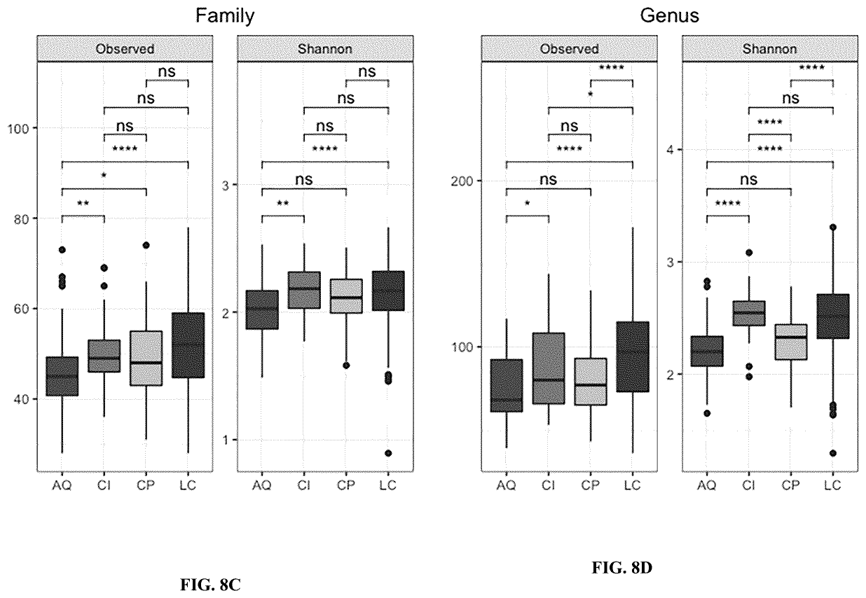

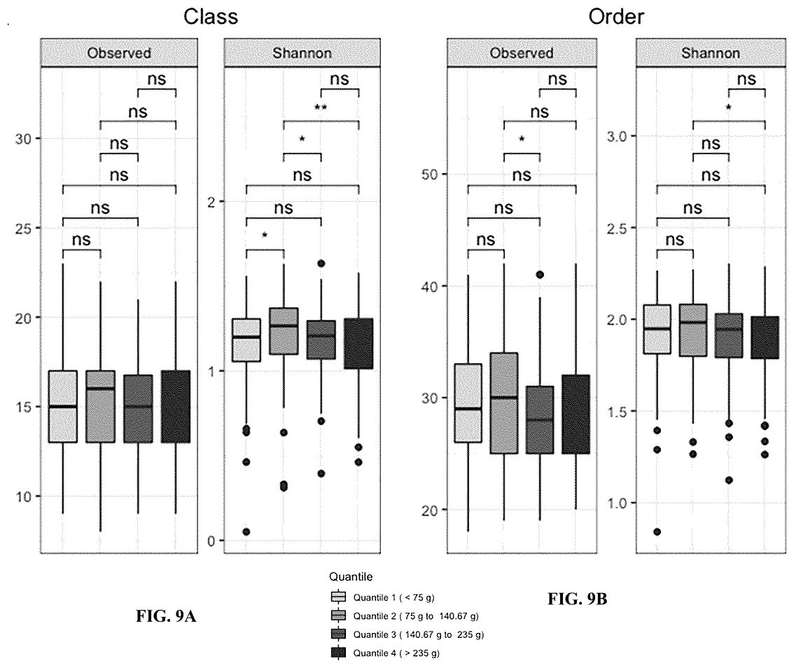

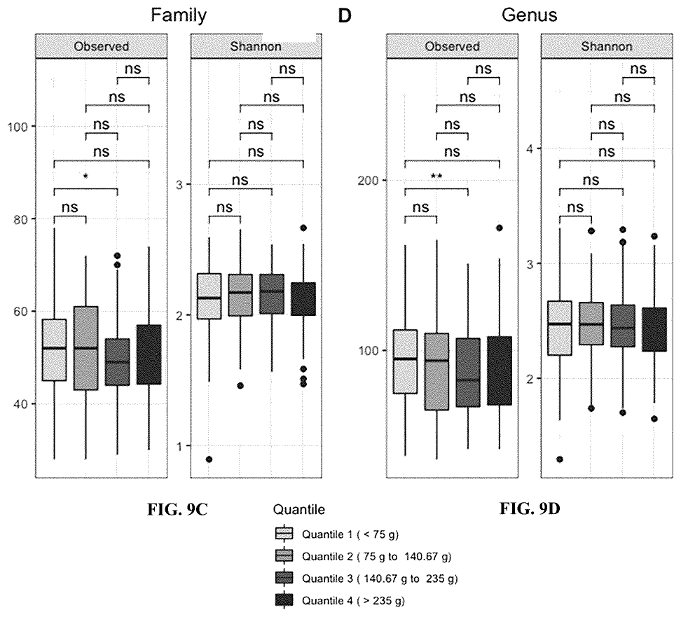

To calculate the complexity of gametophyte microbial communities, taxonomic richness and Shannon diversity of gametophytes was compared between populations and biomass quantiles. For population comparisons, gametophytes were grouped into one of four populations based on where the parent sporophyll was collected: AQ, CI, CP, and LC. When comparing gametophytes from all four populations, taxonomic richness of bacterial species significantly differed across all populations (Kruskal-Wallis, p=0.0016) (FIG. 4). For pairwise comparisons between LC and the remaining populations (AQ, CP, and CI), LC had higher observed richness counts. This trend was similar for higher taxonomic levels (class, order, family, and genus); however, pairwise differences varied in significance (FIG. 8A-FIG. 8D). Shannon diversity also significantly differed across all populations (Kruskal-Wallis, p=1.2e−12) (FIG. 4). Pairwise comparisons of Shannon diversity were all significant except for Arroyo Quemado to Camp Pendleton and Catalina to Leo Carillo. For biomass comparisons, a range of biomass outcomes was considered for adult sporophytes at the time of harvest and four quantile bins were created: Quantile 1 (x≤75 g, n=116), Quantile 2 (75 g>x≤140.67 g, n=113), Quantile 3 (140.67 g>x≤235 g, n=114), and Quantile 4 (x>235 g, n=114). For taxonomic richness between biomass quantiles, there were no significant differences overall regardless of taxonomic level. However, there were some significant differences for taxonomic richness and Shannon diversity for pairwise comparisons between biomass quantiles at the class, order, family, and genus levels (FIG. 9A-FIG. 9D).

### Comparison of Relative Abundance for Each Taxon Across Gametophyte Populations.

The relative abundances of classified taxa (>1%) were calculated at the class, order, family, genus, and species level for all populations together and for each population individually (Table 5). The overall relative abundance for each taxon was calculated using the entire gametophyte dataset (i.e., all populations together). Differences in microbial composition between gametophyte populations were analyzed by subtracting the overall relative abundance from that of each population for every classified taxon. For each taxon, the population that had the largest difference from overall relative abundance was recorded.

### Compositional Normalization and Variance-Based Principal Component Analysis (Beta Diversity).

Absolute abundance counts of bacteria were normalized and transformed using a compositional approach. Briefly, absolute abundance counts were centered log-ratio (clr) transformed using the microbiome package in R. Then, the Aitchison distance, which is the Euclidean distance of dr-transformed counts, for each sample was calculated, then a PCA ordination was performed and visualized with the phyloseq package. To identify whether there were significant differences between group centroids, a PERMANOVA (α=0.05) was performed using the adonis function from the vegan package in R. Significant differences in beta-dispersion was first tested with PERMDISP2 (α=0.05) using the vegan::betadisper function and then further investigated with ANOSIM (α=0.05) using the vegan::anosim function if a significant difference was indicated with the PERMDISP2 test.

### Community Composition Significantly Differs Across Populations and Biomass Quantiles.

The relative abundance of classified bacteria varied across populations (Table 5). The taxa from each population that had the largest difference from overall relative abundance are presented herein. AQ had higher relative abundance of Flavobacteriia, Flavobacteriales, Phyllobacteriaceae, Flavobacteriaceae, and Nonlabens, and lower relative abundance of Alteromonadales, Alteromonadaceae, Brucellaceae, Marinobacter, and Ochrobactrum. CP had higher relative abundance of Alphaproteobacteria, Rhizobiales, Sphingomonadales, Sphingomonadaceae, Erythrobacteraceae, Hoeflea, Sphingorhabdus, and Lentilitoribacter donghaensis, and Sphingopyxis sp., and lower than average relative abundance of Cytophagia, Gammaproteobacteria, Cytophagales, Rhodobacterales, Flammeovirgaceae, and Rhodobacteraceae. LC gametophytes had higher relative abundance of Nonlabens ulvanivorans, Marinobacter sp., and Ochrobactrum sp. (Table 5).

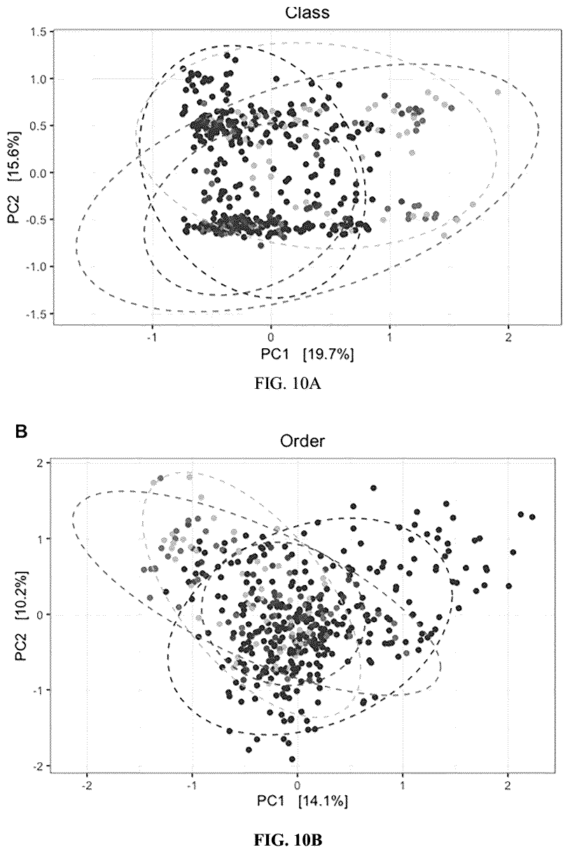

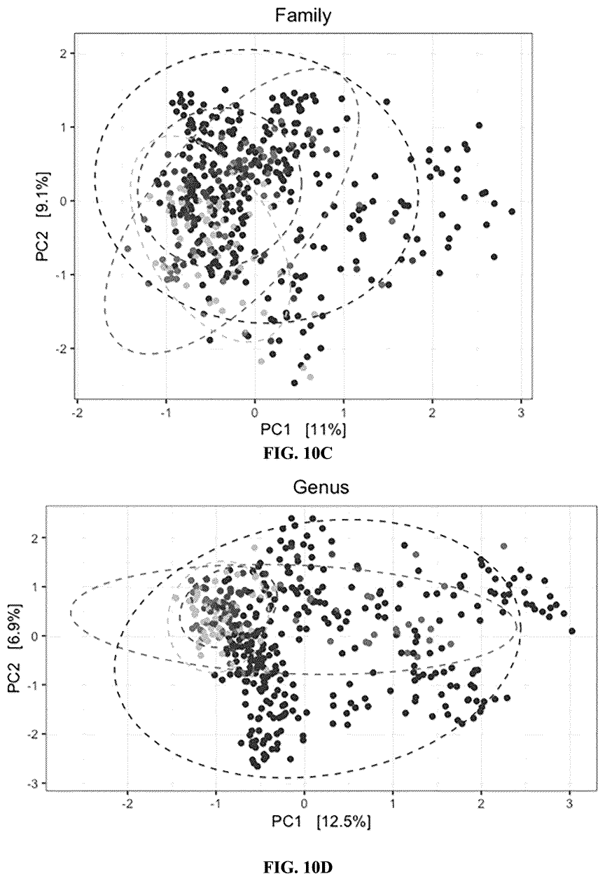

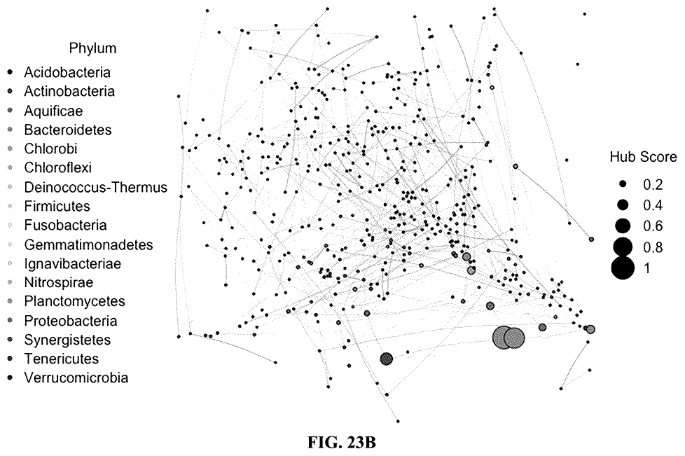

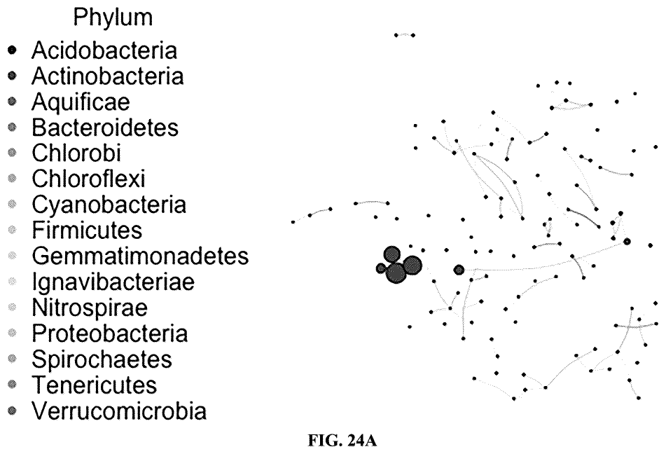

Variance-based compositional principal component analysis (PCA) was performed to visualize differences between microbial community samples (beta diversity). PERMANOVA of gametophyte microbial communities revealed that there was a significant difference in microbial composition between gametophyte populations at the species level (PERMANOVA, p<0.001), with PC1 and PC2 accounting for 18.3% of the variance explained (FIG. 23A-FIG. 23B). This finding was conserved when the microbial communities were classified at the class, order, family, and genus levels (PERMANOVA, p<0.001) with PC1 and PC2 accounting for 35.3%, 24.3%, 20.1%, and 19.4% of the variance explained, respectively (FIG. 10A-FIG. 10D).

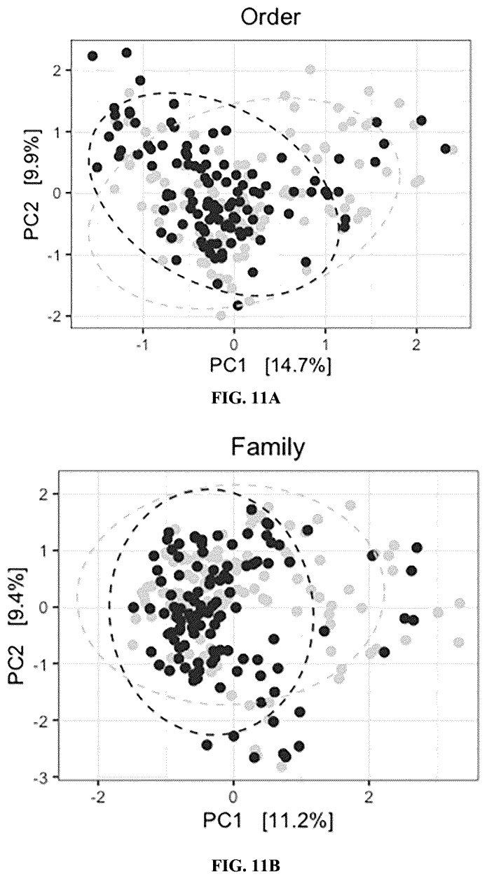

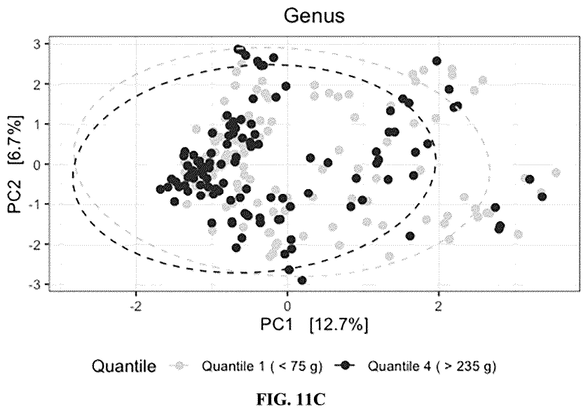

Within-group dispersion was significantly different between populations at all taxonomic levels, with LC having the greatest distance to the centroid at the order, family, genus, and species levels. Distance between groups was not significantly greater than within groups except for class-level microbial community analysis. With LC as the largest sample group, these results are considered conservative. The exception is for analyses done at the class level, in which the smallest group, CI, had the greatest distance to the centroid and results are consequently considered liberal. Looking at a subset of gametophytes that became low and high biomass sporophytes (Quantile 1 (<75 g) and Quantile 4 (>235 g), n=230), there was a significant difference (PERMANOVA, p<0.01) in microbial community composition at the order, family, genus (FIG. 11) and species levels (FIG. 6). The variance explained at these levels was 24.6%, 20.6%, 19.4%, and 19.1%, respectively. There were no significant differences found for within-group dispersion when comparing biomass quantiles.

Differential Abundance of Individual Taxa and Correlation with Sporophyte Biomass.

Differential (relative) abundance and correlation tests were completed using the aldex command within the ALDEx2 package in R, which dr-transforms the data. Only bacteria that had a relative abundance count >1% were included. This analysis was performed on a subset of Macrocystis pyrifera individuals with the highest and lowest sporophyte biomass from the top and bottom biomass quantiles (Table 1). The differential abundance of taxa was compared between the two biomass groups using a Wilcoxon t-test (α=0.1), which was run with the base R function wilcox.test using default settings. Highest and lowest biomass comparisons for differential abundance testing were made for each population separately and all together. For correlation testing, the continuous biomass data for all individuals was used, and the Spearman correlation (α=0.1) between the sporophyte biomass and individual taxa abundance was estimated using ALDEx2. Q-values, which are the Benjamini Hochberg multiple testing corrected p-values, were reported for the Wilcoxon t-test and Spearman correlation (α=0.1). Taxa of interest (q≤0.1) from differential abundance and correlation testing were used to build a linear regression model predicting sporophyte biomass as a factor of dr-transformed relative abundance. The glm and stepAIC functions within the MASS package were used to determine best model fit and significance (t-test, α=0.1; Table 8).

Key Taxa are Differentially Abundant Between Quantile Groups and Correlate with Biomass.

When comparing bacterial taxa between the top and bottom biomass quantiles (Quantile 1 (<75 g) and Quantile 4 (>235 g), n=230), several taxa at the class, order, family, genus, and species levels were found to be differentially abundant (Wilcoxon t-test, q≤0.1; Table 6). The following taxa were found to have increased relative abundance in gametophytes that became high biomass sporophytes: Clostridia, Rhizobiales, Sphingomonadales, Thermoanaerobacterales, Family III, Mesorhizobium, Caldicellulosiruptor, Nitratireductor sp., Zoogloea ramigera, Mesorhizobium genosp., Aquamicrobium sp., and Mesorhizobium sp. The only taxa that had increased relative abundance in gametophytes that became low-biomass sporophytes was Labrenzia sp.

Using a Spearman correlation test on the continuous biomass data and microbial clr-transformed relative abundance for all gametophyte samples (n=457), a number of taxa were identified that correlated (Spearman, q≤0.1) with sporophyte biomass (Table 7). Verrucomicrobiae, Clostridia, Rhizobiales, Sphingomonadales, Thermoanaerobacterales, Erythrobacteraceae, Phyllobacteriaceae, Brucellaceae, Family III, Caldicellulosiruptor, Mesorhizobium, Zoogloea ramigera, Nitratireductor sp., Mesorhizobium genosp., Aquamicrobium sp., and Mesorhizobium sp. each had a positive correlation with sporophyte biomass. Cytophagia and Labrenzia sp. each had a negative correlation with sporophyte biomass.

Select Taxa from the Gametophyte Microbiome are Significant Predictors of Sporophyte Biomass.

To determine which taxa were predictors of biomass, linear regression models were built and iterated for best fit with the taxa identified in differential abundance and correlation testing (Table 6, Table 7, and Table 8). Several taxa from the gametophyte germplasm cultures were significant predictors of sporophyte biomass (GLM t-test, p<0.1), (Table 9). Verrucomicrobiae, Thermoanaerobacterales, Sphingomonadales, Brucellaceae, Mesorhizobium, and Mesorhizobium sp. were predictors of increased sporophyte biomass while Cytophagia and Labrenzia sp. were predictors of decreased biomass. Coefficient estimates ranged from −6.110 to 28.859, with Brucellaceae having the largest value.

### DISCUSSION

Analyses of the most abundant taxa revealed a core microbiome of Macrocystis pyrifera gametophyte germplasm cultures shared across populations (Table 5, FIG. 7A-FIG. 7B). Bacteria that constituted this core microbiome contributed to a set of physiological functions, ecological roles, or symbiotic associations with M. pyrifera gametophytes that were important for normal growth and development. Encouragingly, Alphaproteobacteria, Gammaproteobacteria, Flavobacteriaceae, Rhodobacteraceae, and Alteromonadaceae were also highly abundant in wild M. pyrifera sporophytes collected from Arroyo Quemado, suggesting that bacteria from these classes and families found in the gametophyte germplasm represent a group of important microbes that are conserved across life stages and growth conditions of Southern California giant kelps. Furthermore, Alphaproteobacteria, Flavobacteriia, Gammaproteobacteria, Rhizobiales, Sphinomonadales, Flavobacteriales, Rhodobacteriales, and Alternomonadales were also highly abundant in wild kelp forests with co-occuring M. pyrifera and Nereocystis luetkeana populations sampled in Washington, suggesting that these taxa were conserved in giant kelp populations across the west coast. Of interest, it appears that Cytophagia, Cytophagales, Phyllobacteriaceae, Sphingomonadaceae, Brucellaceae, and Erythrobacteraceae were more abundant in the present dataset; this is a feature either of lab-grown M. pyrifera or of gametophytes more broadly.

Taxonomic richness and Shannon diversity significantly varied by population, with Leo Carillo often having higher values (FIG. 4). Leo Carillo is a putative genetic hybrid zone, and increased microbial species richness and variation is often a product of shared ancestry between kelp populations. The observed richness per individual only represents a small fraction of the total microbial diversity from the full dataset (Table 3). A small set of important taxa present in Macrocystis pyrifera gametophyte cultures and a relatively large number of non-essential taxa were present that varied between microbial communities. Note that the sequencing and classification methods did not account for unobserved taxa that were present in this environment.

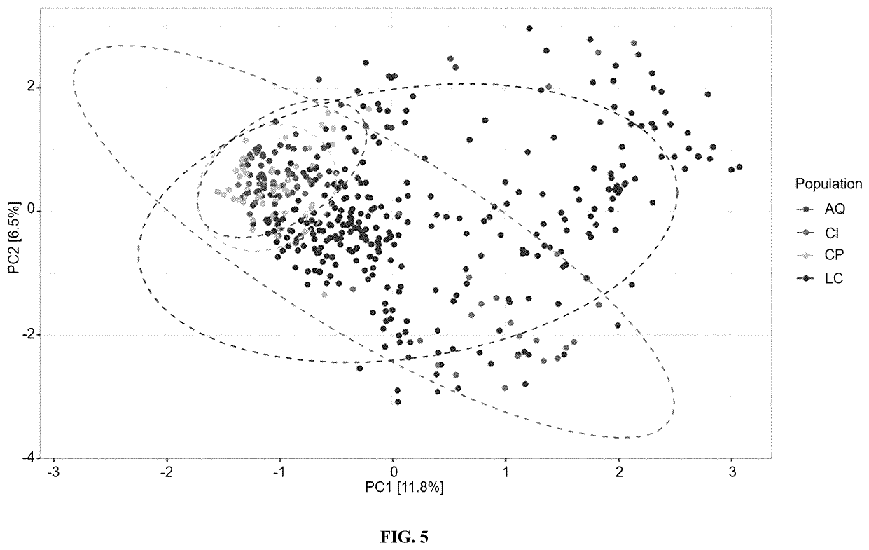

A core microbiome was found by identifying highly abundant taxa present in the gametophyte germplasm that represents several Macrocystis pyrifera populations in Southern California and comparing the findings to M. pyrifera sporophytes collected across the West Coast. Although there was a group of common taxa shared between M. pyrifera individuals, significant differences were found in the composition of the gametophyte dataset when comparing populations and biomass quantiles (FIG. 5 and FIG. 6). The relative abundance of individual taxa varied by population (Table 5). The geographic origin of the parent sporophyte plays a role in microbial community composition, in part by vertical transmission of microbes. The greater dispersion in Leo Carillo is partly a feature of inherited recruitment of microbes, briefly mentioned above, resulting in greater variation of microbial communities. Significant community differences between gametophytes that become high- or low-biomass sporophytes support the determination that the presence of certain taxa and the overall composition play a role in the final yield of cultivated M. pyrifera. Low-biomass sporophytes were more often fouled and covered with visible epiphytes; the gametophyte microbial communities from these sporophytes impacted susceptibility to disease. While the effect of M. pyrifera genetic variation on sporophyte biomass cannot be ignored, the fact that microbial community differences accounted for at least 19.1% of the total variation in biomass shows that microbial inoculants in germplasm cultures increase the yield of M. pyrifera cultivars and provide disease resistance.

Development of a microbial inoculants to be introduced at the gametophyte stage to increase sporophyte biomass included identification of individual species associated with high biomass individuals. Differential abundance and correlation models identified Mesorhizobium, Caldicellulosiruptor, Nitratireductor, Aquamicrobium, and Zoogloea ramigera as promising candidates (Table 10). Indeed, further analysis with a generalized linear model revealed that species within the Mesorhizobium genus were significant predictors of increased sporophyte biomass (Table 8 and Table 9), supporting selection of these species as excellent candidates to use as growth-promoting inoculants. While Brucellaceae had the highest coefficient estimate associated with increased biomass, several species within this family cause disease in humans and studies are performed with appropriate caution.

Regarding their use as a growth-promoting inoculant, Mesorhizobium spp. are known for their nitrogen fixation capabilities. Nitrogen is an essential, limiting compound for plant growth and is increased through the presence of nitrogen fixing bacteria. Nitrogen fixation converts atmospheric nitrogen into a usable and readily available form for plant uptake. Inoculation with Mesorhizobium is an established practice in several crops including legumes, wheat, and chickpea and has shown growth-promoting properties. Although a well-studied mechanism of this phenomena is nitrogen fixation, other factors, such as increased access to nutrients, secretion of plant hormones, and disease resistance, may contribute to the growth-promoting effect.

While the method of bacterial classification used in this study has limited resolution at the species level, this is overcome by culturing gametophyte microbiota, isolating colonies, and doing inoculation trials with species that are classified within the appropriate genus. As the catalog of microbial marine species improves, the dataset used for this study is reanalyzed with updated databases.

Culturing marine bacteria in lab settings is challenging and may be a barrier to development of growth-promoting inoculants. In this case, variations in the kelp genome impact presence or absence of specific microbes, as evidenced by performing a genome-wide association study and designing kelp cultivars genetically that recruit beneficial bacteria. In addition to the development of growth-promoting inoculants, removal of taxa (i.e., Cytophagia and Labrenzia sp.) associated with lower biomass of cultivated seaweeds prevents an inhibiting effect. The current lack of species-specific antibiotics presents an obstacle for removing individual taxa. Solutions include inoculating gametophytes with taxa that compete and outgrowing those associated with lower biomass.

Farmed seaweed cultivars are raised in different environments throughout their lifecycle. Gametophytes are first raised in lab cultures, then transferred to offshore farms as juvenile sporophytes, which impacts microbial community composition. This impacts growth of the seaweed host over time. When a microbial inoculant at the gametophyte stage successfully colonizes and persists through the sporophyte stage, the inoculant results in a growth-promoting effect with minimal changes to the rest of the community and promotes recruitment of other organisms through microbial succession. To fully explore the range of impacts either of these scenarios has on crop outcomes, inoculation trials are performed on Macrocystis pyrifera gametophytes and changes in growth rate and microbial community composition are tracked throughout the cultivar's life cycle.

In conclusion, microbiomes of Macrocystis pyrifera gametophyte germplasm cultures collected from distinct natural populations across Southern California were characterized. These gametophytes were cultivated in an offshore farm in Santa Barbara, California, and the microbial composition of gametophyte cultures varied with the final biomass yield of adult sporophytes. The present disclosure describes technological breakthroughs that leverage recent developments in seaweed genomics resources to develop a set of taxa that increases biomass production of kelp cultivars. These bacterial taxa are tested using a manipulative approach for their capacity as growth-promoting inoculants. The use of nitrogen-fixing Mesorhizobium species promote growth in farmed seaweeds. An inoculant design is effective either by introducing individual bacteria or by introducing a multi-species community. The efficacy of these inoculants is improved by ample consideration of community dynamics and network topology of the existing microbiome to reduce risk of niche competition and to ensure that inoculants are integrated on a large scale. Finally, genetic variation in M. pyrifera cultivars impacts the recruitment of microbes and is considered during inoculant design. The development of a growth-promoting inoculant for M. pyrifera cultivars revolutionizes kelp farming for biofuels. By increasing the biomass yield of individual cultivars, growth-promoting inoculants increase the efficiency of offshore farms and increase the amount of biomass available per harvest. Overall, this disclosure presents an exciting step forward in manipulating the microbial community of M. pyrifera gametophyte germplasm cultures and improving the productivity of offshore farms.

Table 1 includes a description of M. pyrifera biomass quantiles for all populations and for each population individually. Average total biomass was calculated for all surviving sporophyte replicates and divided into quantile bins to investigate group differences in microbial community compositions of corresponding gametophytes. AQ=Arroyo Quemado, CI=Channel Islands, CP=Camp Pendleton, and LC=Leo Carillo.

| | | Quantile 1 | | Quantile 2 | Quantile 3 | Quantile 4 | |
|---|---|---|---|---|---|---|---|
| | Sample | Weight | | Weight | Weight | Weight | |
| Population | Size | (Size) | | (Size) | (Size) | (Size) | |
| All | 457 | <75 | g | 75 g to 140.67 g | 140.67 g to 235 g | >235 | g |
| | | (n = 116) | | (n = 113) | (n = 114) | (n = 114) | |
| AQ | 52 | <70 | g | 70 g to 124 g | 124 g to 240.83 g | >240.83 | g |
| | | (n = 13) | | (n = 13) | (n = 13) | (n = 13) | |
| CI | 44 | <128.42 | g | 128.42 g to 182.67 g | 182.67 g to 246 g | >246 | g |
| | | (n = 11) | | (n = 11) | (n = 11) | (n = 11) | |
| CP | 53 | <160 | g | 160 g to 236.33 g | 236.33 g to 323 g | >323 | g |
| | | (n = 14) | | (n = 13) | (n = 13) | (n = 13) | |
| LC | 308 | <63.92 | g | 63.92 g to 125 g | 125 g to 211 g | >211 | g |

| | | | |
|---|---|---|---|
| (n = 77) | (n = 78) | (n = 76) | (n = 77) |

TABLE 2
Macrocystis pyrifera sampling sites in Southern California. One site per
genetic region identified in the Johansson et al. (2015) genetic differentiation
study was selected. Leo Carrillo, which demonstrated an admixture pattern
from all other regions, was also sampled. Spores released from sampled sporophytes
were used to start the gametophyte germplasm collection.

| Population | Genetic group | Latitude | Longitude | n |
|---|---|---|---|---|
| Arroyo Quemado | Santa Barbara channel | 34.468783° N | −120.121417° W | 60 |
| Catalina Island | Channel Islands | 33.446882° N | −118.485067° W | 60 |
| Camp Pendleton | Southern group | 33.290911° N | −117.499969° W | 60 |
| Leo Carrillo | Hybrid region | 34.042933° N | −118.934500° W | 370 |

Table 3 includes distribution of classified and unclassified taxa across taxonomic levels. Distribution of unique taxa across all 457 Macrocystis pyrifera gametophyte germplasm cultures within the Bacteria domain was classified at five taxonomic ranks: class, order, family, genus, and species. Total number of taxa was estimated using the number of classified taxa and the fraction of unclassified taxa. Four populations of M. pyrifera were represented in this gametophyte set and were named according to their geographic region: Arroyo Quemado (n=52), Channel Islands (n=44), Camp Pendleton (n=53), and Leo Carillo (n=308).

| Taxonomic | Total Taxa | Classified | Fraction |
|---|---|---|---|
| Rank | (est.) | Taxa | Unclassified |
| Class | 54 | 44 | 18.50% |
| Order | 111 | 97 | 12.60% |
| Family | 250 | 205 | 18% |
| Genus | 759 | 626 | 17.50% |
| Species | 1594 | 1106 | 30.60% |

Table 4 includes distribution of taxa across Macrocystis pyrifera populations. Distribution of unique taxa within the Bacteria domain for each population was classified at five taxonomic ranks: class, order, family, genus, and species. Four populations of M. pyrifera were represented in this gametophyte set and were named according to their geographic region: Leo Carillo (n=308), Camp Pendleton (n=53), Channel Islands (n=44), and Arroyo Quemado (n=52).

| | Taxonomic | Total Taxa | Classified | Fraction |
|---|---|---|---|---|
| Population | Rank | (est.) | Taxa | Unclassified |
| Arroyo Quemado | Species | 823 | 571 | 30.60% |
| Arroyo Quemado | Genus | 460 | 365 | 20.70% |
| Arroyo Quemado | Family | 173 | 147 | 15% |
| Arroyo Quemado | Order | 81 | 70 | 13.60% |
| Arroyo Quemado | Class | 38 | 32 | 15.80% |
| Channel Islands | Species | 841 | 593 | 29.50% |
| Channel Islands | Genus | 447 | 360 | 19.50% |
| Channel Islands | Family | 164 | 136 | 17.10% |
| Channel Islands | Order | 69 | 61 | 11.60% |
| Channel Islands | Class | 34 | 29 | 14.70% |
| Camp Pendleton | Species | 844 | 569 | 32.60% |
| Camp Pendleton | Genus | 482 | 386 | 19.90% |
| Camp Pendleton | Family | 176 | 149 | 15.30% |
| Camp Pendleton | Order | 78 | 69 | 11.50% |
| Camp Pendleton | Class | 37 | 31 | 16.20% |
| Leo Carillo | Species | 1480 | 1013 | 31.60% |
| Leo Carillo | Genus | 724 | 598 | 17.40% |
| Leo Carillo | Family | 238 | 197 | 17.20% |
| Leo Carillo | Order | 109 | 95 | 12.80% |
| Leo Carillo | Class | 53 | 43 | 18.90% |

Table 5 describes relative abundance of taxa across Macrocystis pyrifera populations, including relative abundance of taxa (>1%) for each population and overall. LC=Leo Carillo, CP=Camp Pendleton, CI Channel Islands, AQ=Arroyo Quemado.

| Taxonomic | | Over- | | | | |
|---|---|---|---|---|---|---|
| Level | Taxa | all | AQ | CI | CP | LC |
| Class | Alphaproteobacteria | 71.1 | 73.6 | 71.4 | 84.5 | 68.4 |
| Class | Flavobacteriia | 12.2 | 15.4 | 9.4 | 9.3 | 12.5 |
| Class | Cytophagia | 7.8 | 7.1 | 12.9 | 2 | 8.3 |
| Class | Gammaproteobacteria | 7.5 | 3.7 | 5.2 | 3.6 | 9 |
| Order | Rhizobiales | 38.9 | 39.2 | 37.7 | 40.8 | 38.7 |
| Order | Sphingomonadales | 22.3 | 30.3 | 23.1 | 38.2 | 18.3 |
| Order | Flavobacteriales | 12.2 | 15.4 | 9.4 | 9.3 | 12.5 |
| Order | Cytophagales | 7.8 | 7.1 | 12.9 | 2 | 8.3 |
| Order | Rhodobacterales | 7.4 | 3.7 | 8.9 | 3.3 | 8.5 |
| Order | Alteromonadales | 4.4 | 2.2 | 2.9 | 2.4 | 5.3 |
| Family | Phyllobacteriaceae | 31.3 | 35 | 31.1 | 36.3 | 29.9 |
| Family | Sphingomonadaceae | 19.1 | 25.5 | 21.4 | 27.3 | 16.4 |
| Family | Flavobacteriaceae | 11.8 | 15.2 | 12.9 | 9.3 | 11.9 |
| Family | Flammeovirgaceae | 7.7 | 7 | 9.4 | 2 | 8.3 |
| Family | Rhodobacteraceae | 7.4 | 3.7 | 8.9 | 3.3 | 8.5 |
| Family | Alteromonadaceae | 4.2 | 1.9 | 2.7 | 2.2 | 5.1 |
| Family | Brucellaceae | 3.3 | 2.6 | 3.8 | 3.3 | 3.4 |
| Family | Erythrobacteraceae | 3.3 | 4.9 | 1.7 | 10.9 | 1.9 |
| Genus | Hoeflea | 29.7 | 32.9 | 27.5 | 34.7 | 28.6 |
| Genus | Sphingorhabdus | 18.6 | 24.2 | 21 | 26.1 | 16.2 |
| Genus | Nonlabens | 5.5 | 10 | 4 | 7.9 | 4.5 |
| Genus | Marinobacter | 3.9 | 1.8 | 2.7 | 2 | 4.7 |
| Genus | Ochrobactrum | 3.3 | 2.6 | 3.7 | 3.2 | 3.4 |
| Species | Lentilitoribacter | 29.6 | 32.8 | 27.3 | 34.6 | 28.5 |
| | donghaensis | | | | | |
| Species | Sphingopyxis sp. | 18.1 | 24 | 20.2 | 25.9 | 15.7 |
| Species | Nonlabens | 5.3 | 9.6 | 3.9 | 7.5 | 15.7 |
| | ulvanivorans | | | | | |
| Species | Marinobacter sp. | 3.8 | 1.7 | 2.6 | 1.9 | 15.7 |
| Species | Ochrobactrum sp. | 3.3 | 2.6 | 3.7 | 3.2 | 15.7 |

Table 6 lists taxa differentially abundan tbetween sporophyte biomass groups, including taxa (>100 relative abundance) that were differentially abundant (q≤0.1) between the top and bottom biomass quantiles (Quantile 1 (<75 g) and Quantile 4 (>235 g), n=230) for gametophyte germplasm cultures. A positive effect size indicated that the taxon was more abundant in gametophytes that became high biomass sporophytes. A negative effect size indicated that the taxon was more abundant in those that became low-biomass sporophytes. Q-values are the Benjamini Hochberg corrected p-values for multiple testing.

| Taxonomic | | Effect | Q- |
|---|---|---|---|
| Level | Taxa | Size | Value |
| Class | Clostridia | 0.2 | 0.1 |
| Order | Rhizobiales | 0.19 | 0.1 |
| Order | Sphingomonadales | 0.22 | 0.05 |
| Order | Thermoanaerobacterales | 0.27 | 0.02 |
| Family | Family III (Thermoanaerobacterales) | 0.26 | 0.04 |
| Genus | Mesorhizobium | 0.23 | 0.01 |
| Genus | Caldicellulosiruptor | 0.24 | 0.07 |
| Species | Labrenzia sp. | −0.28 | 0.01 |
| Species | Nitratireductor sp. | 0.15 | 0.1 |
| Species | Zoogloea ramigera | 0.15 | 0.06 |
| Species | Mesorhizobium genosp. | 0.19 | 0.02 |
| Species | Aquamicrobium sp. | 0.2 | 0.02 |
| Species | Mesorhizobium sp. | 0.22 | 0.01 |

Table 7 lists taxa (>1% relative abundance) that significantly correlated with sporophyte biomass. A Spearman correlation was used to model the association between individual taxa at the gametophyte stage with sporophyte biomass. All gametophyte samples (n=457) and their continuous biomass data were used. Q-values are the Benjamini Hochberg corrected p-values for multiple testing. Only results with q≤0.1 are shown.

| Taxonomic Level | Taxa | Spearman Rho | Q-Value |
|---|---|---|---|
| Class | Cytophagia | −0.1 | 0.09 |
| Class | Verrucomicrobiae | 0.09 | 0.1 |
| Class | Clostridia | 0.12 | 0.06 |
| Order | Rhizobiales | 0.11 | 0.1 |
| Order | Sphingomonadales | 0.13 | 0.07 |
| Order | Thermoanaerobacterales | 0.17 | 0.01 |
| Family | Erythrobacteraceae | 0.11 | 0.1 |
| Family | Phyllobacteriaceae | 0.11 | 0.1 |
| Family | Brucellaceae | 0.12 | 0.1 |
| Family | Family III | 0.17 | 0.01 |
| Genus | Caldicellulosiruptor | 0.16 | 0.02 |
| Genus | Mesorhizobium | 0.19 | 0.004 |
| Species | Labrenzia sp. | −0.16 | 0.01 |
| Species | Zoogloea ramigera | 0.14 | 0.03 |
| Species | Nitratireductor sp. | 0.15 | 0.02 |
| Species | Mesorhizobium genosp. | 0.16 | 0.01 |
| Species | Aquamicrobium sp. | 0.17 | 0.01 |
| Species | Mesorhizobium sp. | 0.18 | 0.004 |

Table 8 describes general lineal models (GLMs) used to model microbe abundance and sporophyte biomass, including linear regression models at the class, order, family, genus, and species levels. Models were first built using the list of taxa identified in differential abundance and correlation testing (Tables 2 and 3), then predictor variables were chosen using the stepAIC method to improve model fit.

| Taxonomic | |
|---|---|
| Level | Model |
| Class | Biomass ~ Cytophagia + Verrucomicrobiae |
| Order | Biomass ~ Thermanaerobacterales + Sphingomonadales |
| Family | Biomass ~ Family III (Thermanaerobacterales) + |
| | Brucellaceae |
| Genus | Biomass ~ Mesorhizobium |
| Species | Biomass ~ Mesorhizobium sp. + Labrenzia sp. |

Table 9 summarizes existing knowledge regarding taxa of interest (non-exhaustive). Column descriptions (left to right) include: (1) taxonomic level for taxa of interest, (2) taxa identified in this study that were associated with Macrocystis pyrifera sporophyte biomass, (3) positive or negative association with biomass as modeled in this study, (4) general associations with plant growth described in previous studies, (5) brief review of known functions.

| | | Association | Association | |
|---|---|---|---|---|
| Taxonomic | | w/M. pyrifera | w/plant | |
| Level | Taxa | biomass | growth | Functional highlights |
| Class | Cytophagia | Positive | Unknown | Remineralize organic compounds into |
| | | | | micronutrients |
| Class | Clostridia | Positive | Positive | Fermentation of polysaccharides |
| Class | Verrucomicrobiae | Positive | Positive | Carbon and nitrogen cycling, |
| | | | | polysaccharide degradation |
| Order | Rhizobiales | Positive | Positive | Nitrogen fixation, provide nutrients, |
| | | | | phytohormones, and pre-metabolites |
| Order | Sphingomonadales | Positive | Positive | Degrade aromatics, environmental |
| | | | | remediation |
| Order | Thermoanaero- | Positive | Positive | Biohydrogen production |
| | bacterales | | | |
| Family | Brucellaceae | Positive | Unknown | Pathogenic in mammals |
| Family | Erythrobacteraceae | Positive | Positive | Carbon cycling and energy metabolism |
| Family | Family III | Positive | Positive | Biohydrogen production |
| | (Thermoanaero- | | | |
| | bacterales) | | | |
| Family | Phyllobacteriaceae | Positive | Positive | Nitrogen fixation |
| Genus | Aquamicrobium | Positive | Unknown | Oxidase and catalase activity |
| Genus | Caldicellulosiruptor | Positive | Unknown | Lignocellulose degradation |
| Genus | Mesorhizobium | Positive | Positive | Nitrogen fixation |
| Genus | Nitratireductor | Positive | Unknown | Nitrate reduction |
| Species | Labrenzia sp. | Negative | Unknown | Antibacterial activity |
| Species | Zoogloea ramigera | Positive | Unknown | Biosorption of metals, formation of |
| | | | | activated sludge flocs |

Table 10 describes coefficient estimates from GLM modeling microbe abundance and sporophyte biomass, including taxa, coefficient estimates, standard errors, and p-values of predictor variables (taxa of interest) for sporophyte biomass. Coefficient estimates represent the average change in the log odds value of increased sporophyte biomass with a one unit increase in the dr-transformed abundance of select taxa.

| Taxonomic | | Coefficient | Std. | P- |
|---|---|---|---|---|
| Level | Taxa | Estimate | Error | Value |
| Class | Cytophagia | −5.275 | 2.412 | 0.0293 |
| Class | Verrucomicrobiae | 9.648 | 5.825 | 0.0984 |
| Order | Thermoanaerobacterales | 6.658 | 3.064 | 0.0303 |
| Order | Sphingomonadales | 19.335 | 7.83 | 0.0139 |
| Family | Brucellaceae | 28.859 | 13.072 | 0.0278 |
| Genus | Mesorhizobium | 14.009 | 3.798 | 0.00025 |
| Species | Mesorhizobium sp. | 13.428 | 3.511 | 0.00015 |
| Species | Labrenzia sp. | −6.11 | 2.18 | 0.00528 |

### Example 2

### Production of Gametophytes and Cultivation of Sporophytes.

Sporophyte collection, spore release, sequencing, and classification is briefly described here. Reproductive blades of M. pyrifera were collected from Southern California regions in December 2018 representing four genetically distinct natural populations: Arroyo Quemado (AQ), Catalina Island (CI), Camp Pendleton (CP), and Leo Carillo (LC). Blades were shipped overnight to University of Wisconsin-Milwaukee for spore release in sterile Provasoli enriched seawater medium (PES) at 34 PSU salinity following the Oppliger method. Spores were raised to the gametophyte stage, then isolated and vegetatively grown to create genetically unique germplasm cultures. From this germplasm, a total of 2,500 sporophytes (500 unique genotypes with five replicates each) were produced from the cross of 500 unique female gametophytes (370 from LC, and 60 each from AQ, CI, and CP) with a single male from LC. Gametophyte crosses were seeded on polyvinyl lines and grown in lab conditions for one month before being shipped overnight to a marine laboratory at the University of California, Santa Barbara (UCSB). Sporophytes were adjacently planted on ten longlines in an offshore farm 1-mile off the coast of Santa Barbara in May 2019. All surviving sporophytes were harvested between Sep. 7-12, 2019 using Santa Barbara Mariculture's vessel Perseverance. Harvested sporophytes were weighed to record total wet biomass, which included stipe and blades. The average biomass of all surviving genetic replicates was calculated and used in this study. A number of individuals were lost due to issues during harvest or premature loss. Due to smaller sample size and restricted availability of phenotype data for the AQ, CI, and CP populations, network analysis across biomass outcomes for the LC population were reported (see: ‘Grouping gametophytes and taxonomy levels for biomass and population comparisons’).

### DNA Extraction, Microbial Shotgun Sequence Data, and Classification.

For DNA extraction, aliquots of each culture were centrifuged and gametophyte tissue was pulverized using liquid nitrogen. Kelp genome and microbial DNA were co-extracted and sequenced from female and male gametophytes using the NucleoSpin 96 Plant Kit (Macherey-Nagel, Duren, Germany). Due to the nature of this extraction technique, the microbial DNA of both exogenous and endogenous species was extracted. For sequencing, 11.2 GB of 150 bp reads per sample was generated at BGI North American NGS lab using an Illumina S4 Novaseq platform. Raw fastq files were processed with the ‘fastp’ program. Due to evidence of bacterial contamination in existing brown macroalgae genomes, all reads were included in the bacterial classification pipeline to ensure that all candidate sequences were analyzed. Reads were classified using the ‘metaxa2’ package (version 2.2.2) which extracts and classifies partial rRNA sequences against the SSU_SILVA128 database. The resulting abundance Tables were further processed and analyzed with the ‘phyloseq’ package in R. Abundance counts were processed by removing singletons and doubletons, normalizing counts by sequencer, averaging counts for samples that were sequenced over multiple runs, and again removing any remaining singletons and doubletons. Only taxa from the bacterial domain were kept for analysis.

### Grouping Gametophytes and Taxonomy Levels for Biomass and Population Comparisons.

Due to the smaller number of individuals within the AQ, CI, and CP populations, the comparison of network analyses across biomass outcomes was performed with individuals from the LC population. A total of 308 individuals from the LC population were divided into one of four quantile groups based on their wet biomass weight at the time of harvest: Quantile 1 (63.92 g, n=77), Quantile 2 (>63.92 g and <125 g, n=78), Quantile 3 (>125 g and <211 g, n=76), and Quantile 4 (>211 g, n=77). These biomass values represent that of diploid sporophytes grown on the farm. Recall that the crossing scheme used in this study crossed a single male gametophyte from the LC population with 500 female gametophytes across the AQ, CI, CP, and LC populations (see: ‘Production of gametophytes and cultivation of sporophytes’). Consequently, the microbial community of the corresponding female gametophyte for each sporophyte was analyzed. After running the bacterial classification pipeline described above, bacterial reads were conglomerated to four taxonomic levels (order, family, genus, and species) for all LC individuals. Analyses at several taxonomic levels were conducted to address the challenge of taxonomic resolution and classification uncertainty at higher levels (i.e. genus and species) and to consider lower levels (i.e. order, and family) as proxies for ecological function. For population comparisons, individuals were grouped according to the geographic region (natural population) in which their parent sporophyte was collected: AQ (n=64, 12 males and 52 females), CI (n=57, 12 males and 45 females), CP (n=69, 16 males and 53 females), and LC (n=369, 54 males and 315 females). Because population comparisons did not include biomass data, a larger number of available individuals were sourced, including a number of males that were not used in the crossing scheme for the farm. However, due to the complexity of the microbial communities for these samples, network analyses were run at the order and family levels.

### Quantification and Visualization of Co-Occurrence Network and Hub Taxa.

Starting with network analysis across biomass outcomes, LC gametophytes (n=308) were divided into four quantiles as described above. For each quantile, 50 individuals were randomly selected 100 times and networks were constructed using the R package ‘SpiecEasi’, which infers ecological associations in microbial communities. The default settings for SpiecEasi with neighborhood selection (the Meinshausen and Buhlmann or “MB” method) were used. The resulting representative network models were analyzed and graphed with the ‘igraph’ package in R. For each network, the following network topology features were recorded: total nodes, total edges, number of positive edges, number of negative edges, ratio of positive to negative edges, average path length, heterogeneity, modularity, average degree per node, clustering coefficient, and hub score. Nodes represent unique taxa and edges are the significant co-occurrences between them. Positive edges indicate that connected taxa tend to be present together and negative edges indicate the opposite (i.e., if one is present in a community, the other is absent). Positive and negative edge information was also used to infer whether taxa of interest had competitive interactions with other taxa. The average path length considers the shortest edge path connecting each pair of nodes. Heterogeneity, the distribution of degrees or connections from each node, was calculated. Modularity, the density of node connections compared to a randomly structured network, was measured with the Louvain method that maximizes the score for each community. Hub score was calculated for the whole network without subsampling using Kleinberg's centrality score, which ranges from 0 to 1. This pipeline was repeated with microbial networks classified at the order, family, genus, and species levels. For network analyses across gametophyte populations, the same pipeline was used, and 50 individuals were randomly selected 100 times from each population: AQ (n=64), CI (n=57), CP (n=69), and LC (369). Due to the higher complexity of these microbial networks and subsampling regime, networks for the LC population were not constructed at the genus and species levels. Therefore, the network analyses done at the order and family levels across all four populations are included herein.

Identifying Network Topology Factors that Predict Sporophyte Biomass.

For network comparisons across biomass quantiles, gametophytes from the LC population (n=308) were divided into one of four quantile groups based on their wet biomass weight at the time of harvest: Quantile 1 (<63.92 g, n=77), Quantile 2 (>63.92 g and <125 g, n=78), Quantile 3 (>125 g and <211 g, n=76), and Quantile 4 (>211 g, n=77). Analysis was repeated with bacteria conglomerated to the order, family, genus, and species levels. Networks were constructed (described above) by randomly selecting 50 individuals 100 times from each quantile group. An ordered logistic regression model was estimated using the ‘polr’ command from the ‘MASS’ package in R. The model was first run using all non-multicollinear factors: total nodes, total edges, positive to negative edge ratio, average path length, modularity, average degree, heterogeneity, and clustering coefficient. Using the ‘regsubsets’ command from the ‘leaps’ package in R, the predictors for host biomass were determined using co-occurrence networks generated from microbiomes classified at the following taxonomic levels: order, family, genus, and species. As stated earlier, analyses at several taxonomic levels were done to address the challenge of taxonomic resolution and classification uncertainty at higher levels (i.e. genus and species) and to consider lower levels (i.e. order, and family) as proxies for ecological function. Models were additionally confirmed for best fit factors using the ‘stepAIC’ command from MASS. In the case of a mismatch, which occurred at the genus and species level, the simpler model was chosen. Log likelihoods were converted to odds ratios for ease of interpretation.

### Network Topology is a Predictive Measure of Sporophyte Biomass.

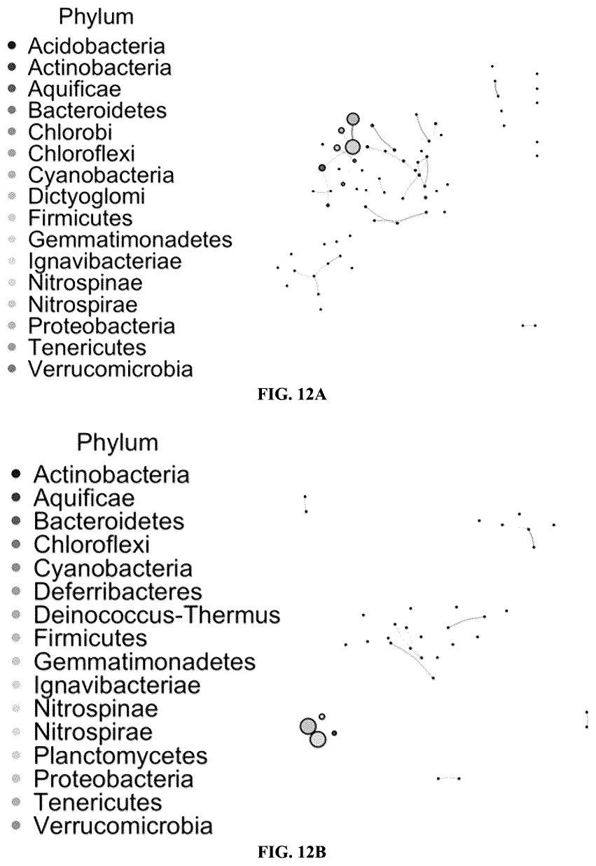

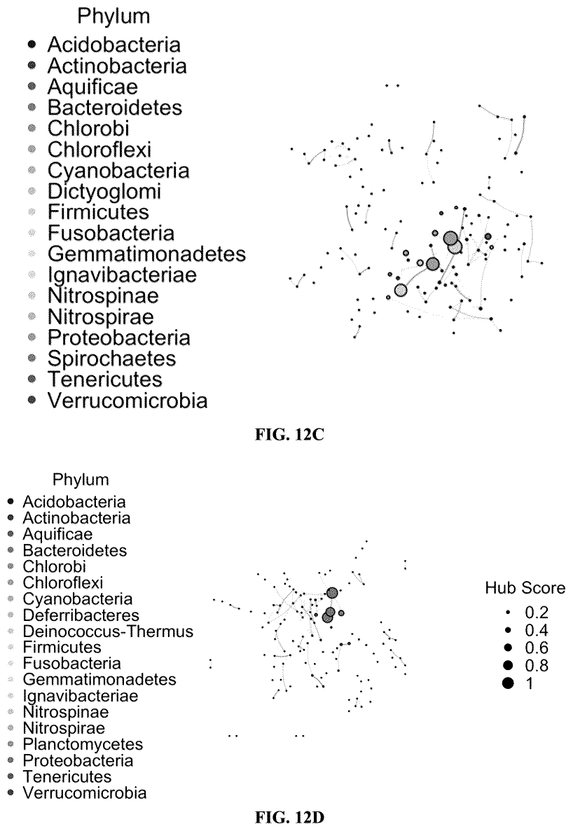

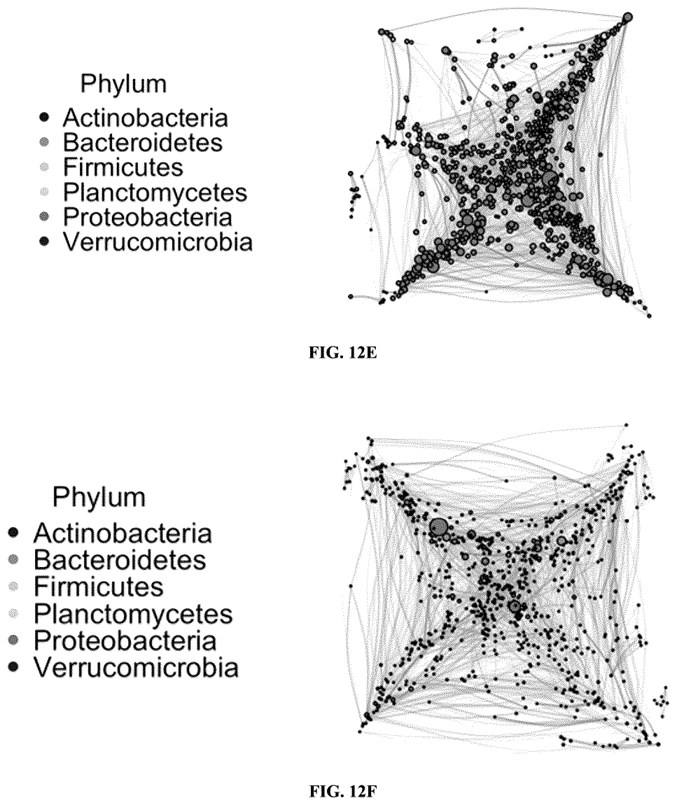

Using gametophytes from the Leo Carillo (LC) population (n=308), co-occurrence networks were constructed of the microbial community with taxa classified at the order, family, genus, and species level (FIG. 23, FIG. 12). LC gametophytes were binned into one of four biomass quantile groups based on their sporophyte weight at the time of harvest. Network analyses were performed for each biomass group and topological measures of the co-occurrence networks of their associated microbiomes were recorded (Table 11). To identify network topology factors that vary with biomass, a proportional odds logistic regression model was used. The best fit model for each taxonomic level included different topology factors (Table 12, Table 13). Clustering coefficient was a strong predictor of biomass across the order, family, genus, and species levels; however, its association with increased biomass changed across levels. At the order and species level, with a one unit increase in the clustering coefficient, the odds of higher biomass were 3.52e+3 and 1.60e+37 more likely, respectively. At the family and genus levels, there was an opposite trend with higher biomass being 4.30e+5 and 4.07e+7 times less likely, respectively. Positive to negative edge ratio was also a predictive factor for biomass at the order, family, and genus levels: with each one unit increase (i.e. a higher proportion of positive associations between taxa), the odds of higher biomass were 1.04, 1.22, and 1.22 times more likely, respectively. Increased heterogeneity and lower modularity were predictive of higher biomass at the order and family levels. For each one unit increase in heterogeneity, increased biomass was 1.00e+9 and 6.34e+9 times more likely. For each one unit increase in modularity, the odds of increased biomass were 1.97e+7 and 4.50e+9 times less likely. Finally, for average path length at the order level with each one unit increase the odds of increased biomass was 1.75 times less likely.

### Comparing Network Topology Measures Between Populations.

For network comparisons across populations, gametophytes were analyzed from four populations: AQ (n=64), CI (n=57), CP (n=69), and LC (369). Networks were constructed by randomly selecting 50 individuals 100 times from each population. Analyses were repeated with bacteria conglomerated to the order and family levels. A Kruskal Wallis test was used to determine if there was a significant difference overall across populations for the following topology measures: total nodes, total edges, ratio of positive to negative edges, average path length, modularity, average degree, heterogeneity, and clustering coefficient. Pairwise comparisons were tested for significant differences using a Wilcoxon test.

Microbial Communities from Gametophytes that Become Low- or High Biomass Sporophytes have Unique Hub Taxa.

From the network analyses described above, hub scores were calculated for each taxon and those with the highest scores were identified (FIG. 23A-FIG. 23B and Table 14). Hub taxa are those that had a score of at least 0.5, which scores were reported from the order, family, genus, and species levels (Table 14). Microbial communities from gametophytes that became low-biomass sporophytes (<63.92 g) had the following hub taxa (score followed in parentheses): orders Frankiales (1) and Kineosporiales (0.89); families Veillonellaceae (1), Archangiaceae (0.99), Burkholderiaceae (0.89), and Clostridiaceae 1 (0.85); genera Marixanthomonas (1), Magnetococcus (0.91), Epibacterium (0.84), alpha proteobacterium PWB3 (0.57), and Collinsella (0.53); species Kordiimonas lacus (1), Methylosinus trichosporium (0.87), alpha proteobacterium SAORIC-651 (0.74), Stappia taiwanensis (0.62), and marine bacterium VA011 (0.55). In general, high biomass sporophytes (>211 g) had fewer hub taxa in the gametophyte microbial communities. High biomass hub taxa were orders Desulfovibrionales (1), Nitrospinales (0.99); families Magnetococcaceae (1), Beijerinckiaceae (0.95), and Holosporaceae (0.81); genera Wenyingzhuangia (1) and Pedobacter (0.89); and species mucus bacterium 80 (1) and Marinomonas brasilensis (0.59).

M. pyrifera gametophytes from the Leo Carillo population were binned into biomass groups based on their sporophyte weight at the time of harvest. Representative networks were generated for the microbial communities of each biomass group. Taxa were then given a score to quantify their role as hub taxa. Taxa from the genus and species levels that scored over 0.5 are recorded here. Biomass groups: Low (<63.92 g, n=77) and High (>211 g, n=77). Taxa names are listed as the direct outputs from the metaxa2 classification pipeline with the SILVA 128 database.

Candidate Growth-Promoting Taxa Co-Occurs with Hub Microbes of Gametophytes that Become High Biomass Sporophytes

Bacteria from the genus Mesorhizobium were associated with increased biomass of M. pyrifera and therefore are a prime component for a growth-promoting inoculant. Using the representative networks constructed for this study, positive and negative associations of Mesorhizobium with other taxa in the microbial community of M. pyrifera gametophyte germplasm cultures were determined. Mesorhizobium co-occurs with Wenyingzhuangia and Pedobacter, which had the two highest hub scores for gametophytes that became high biomass sporophytes. Mesorhizobium had negative co-occurrence values with Aquamarina, Sneathiella, Pseudohaliea, and Saccharospirillum.

### Network Topology and Hub Taxa Differs Between Gametophyte Populations.

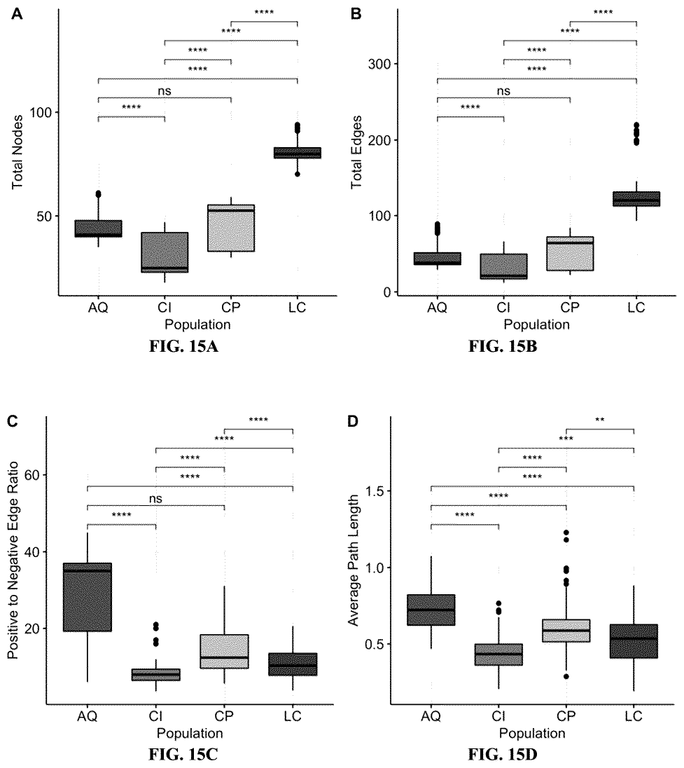

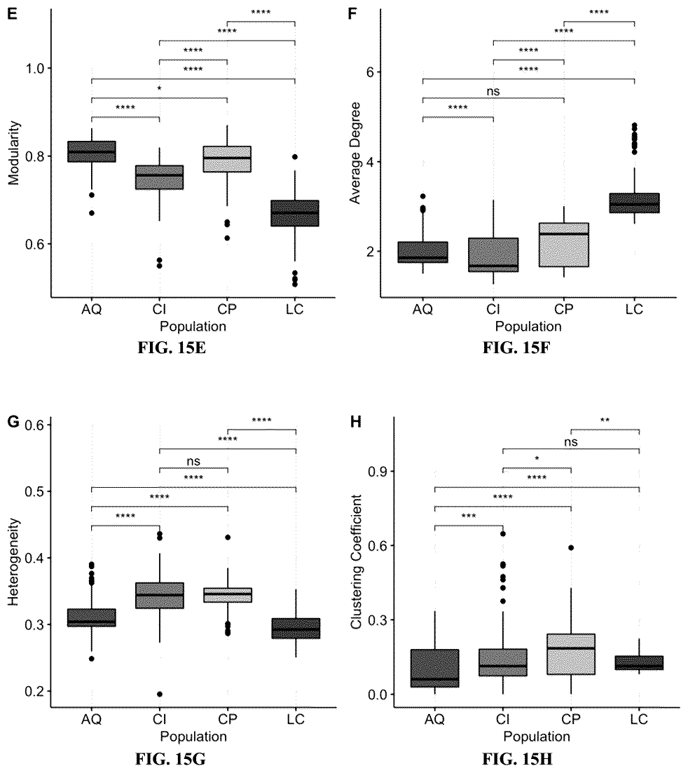

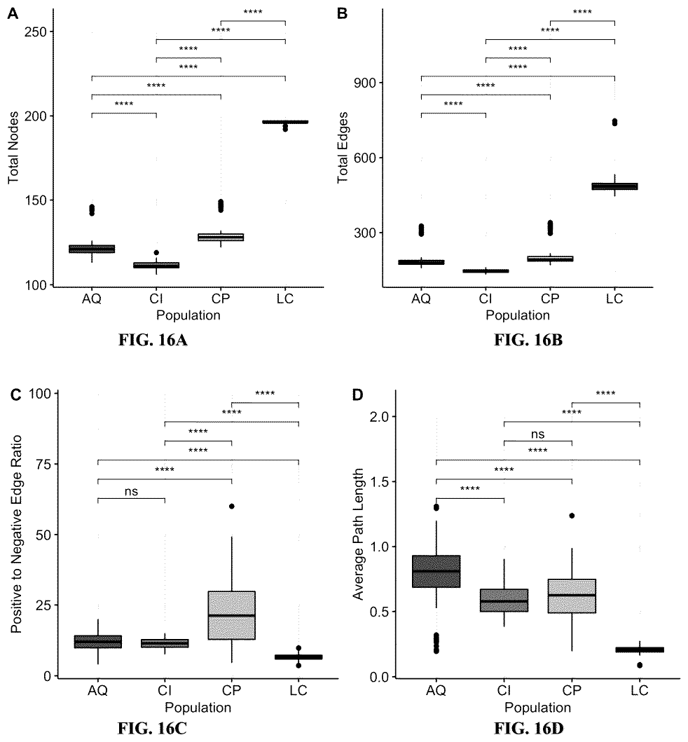

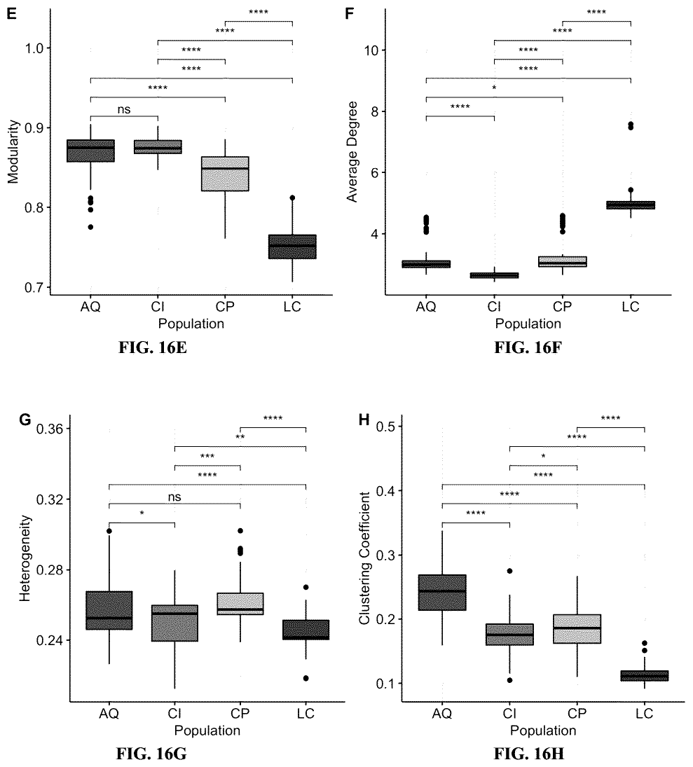

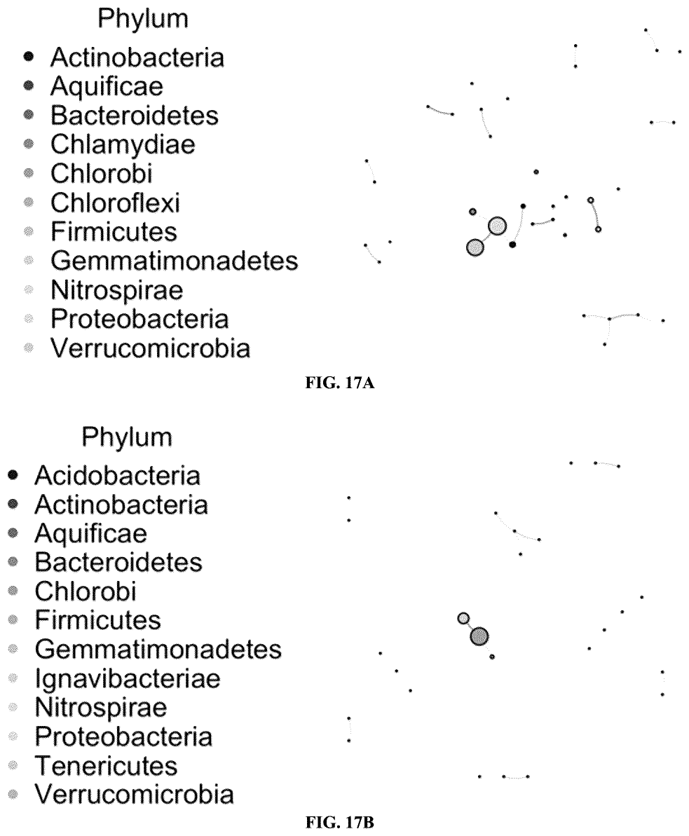

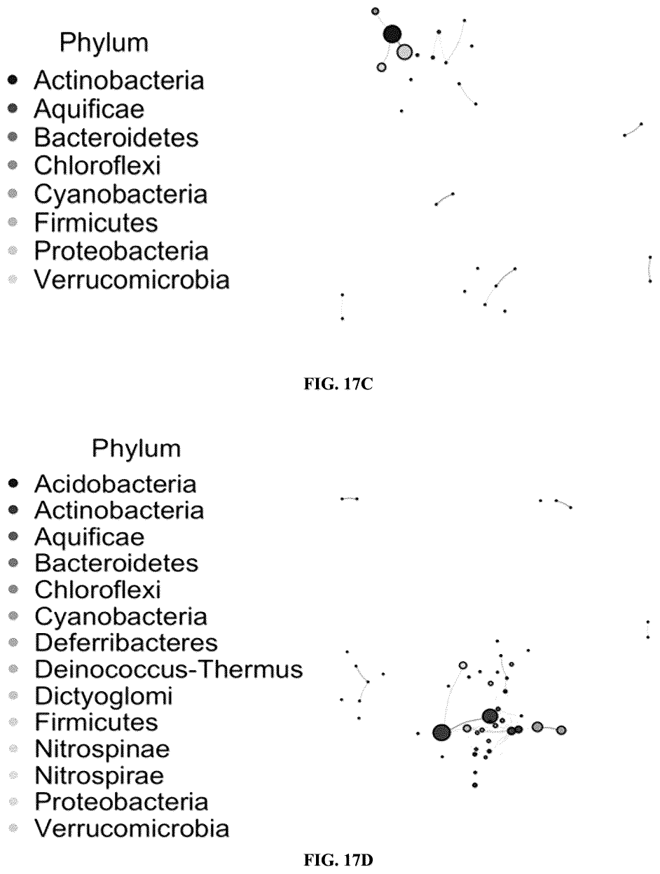

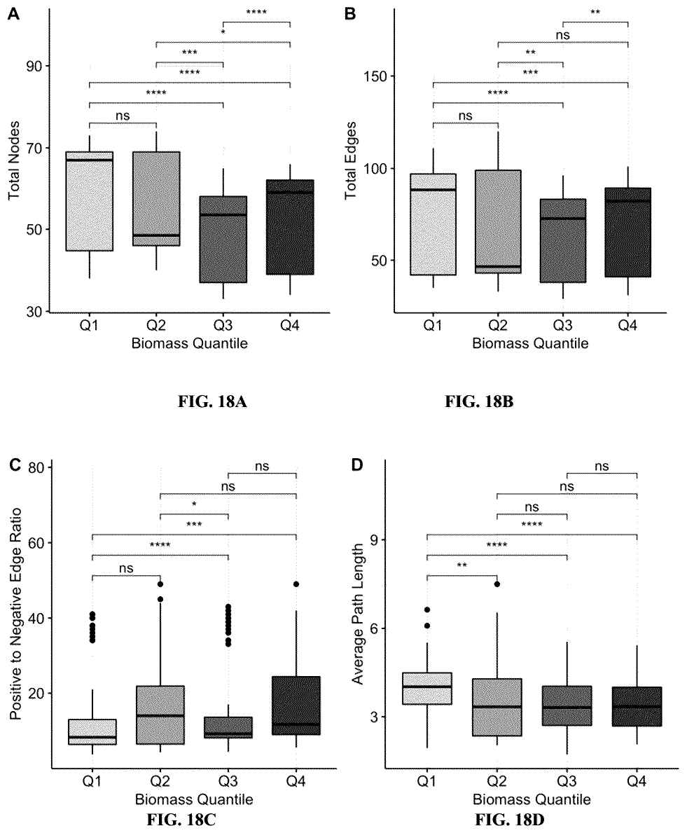

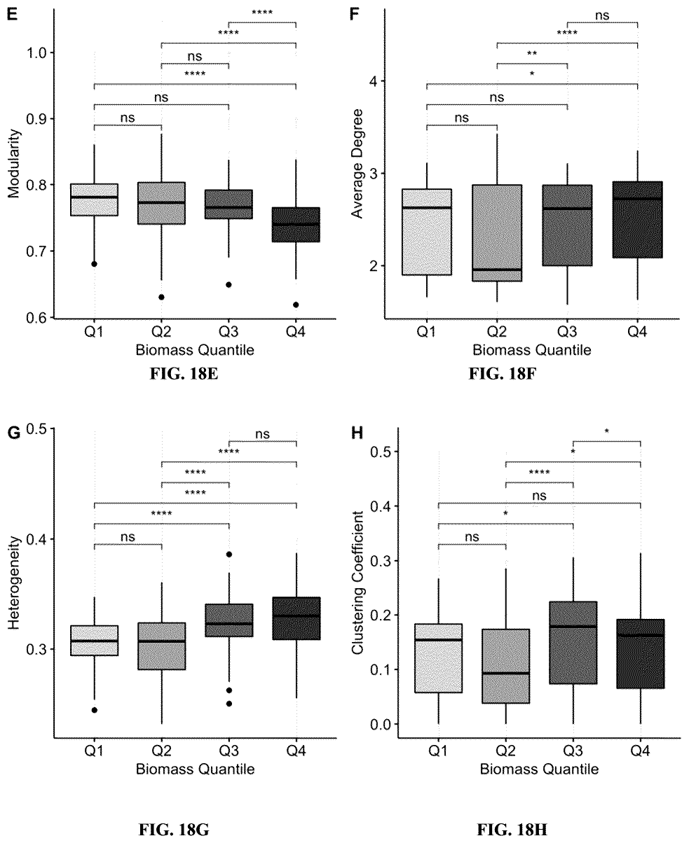

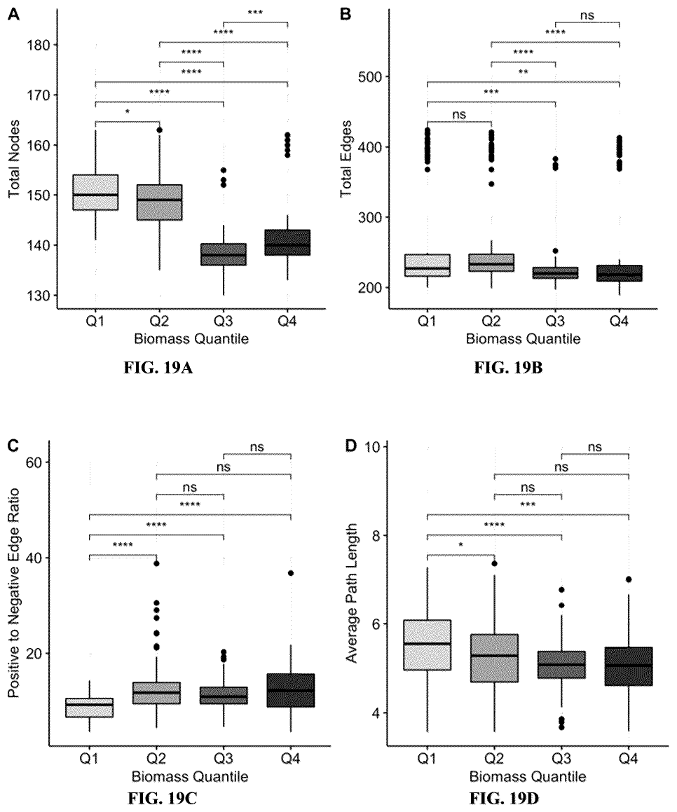

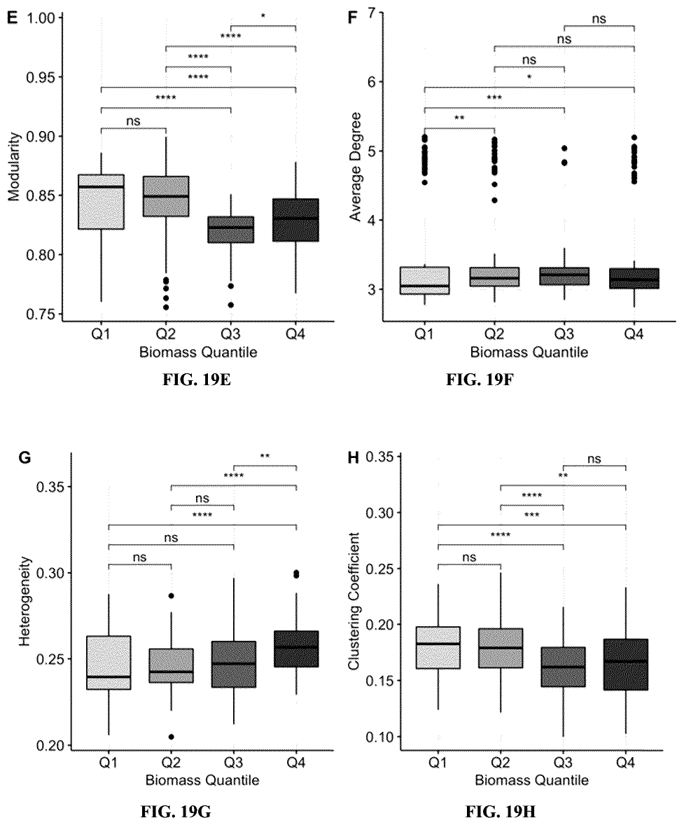

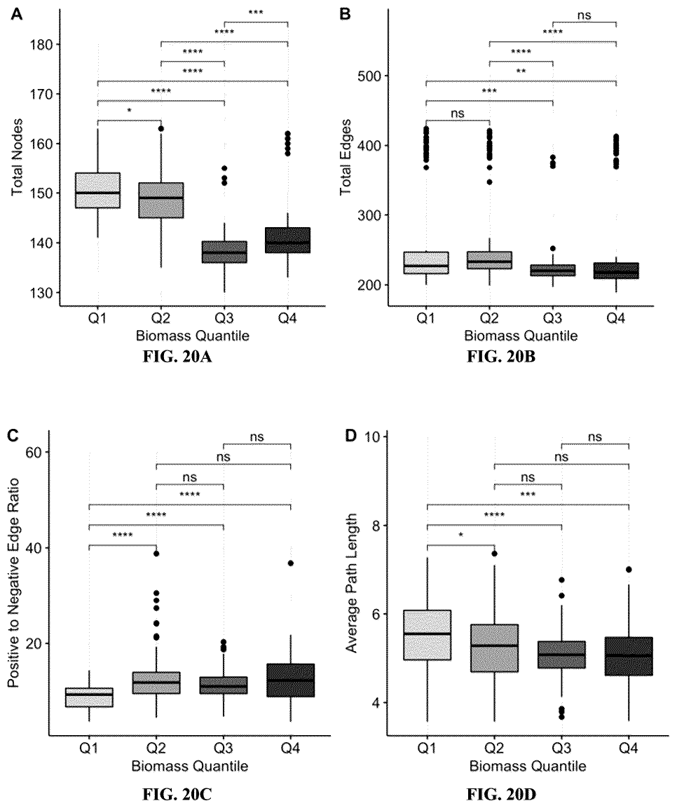

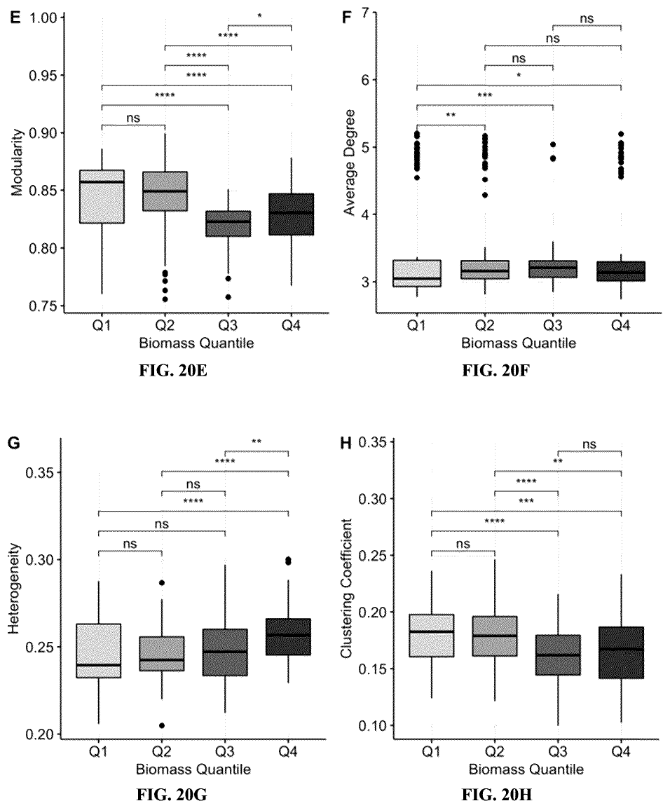

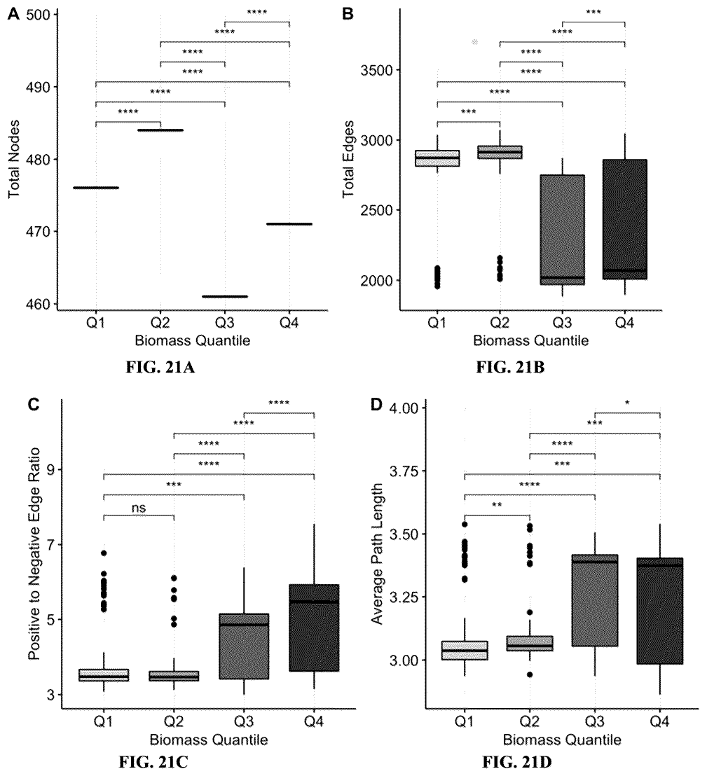

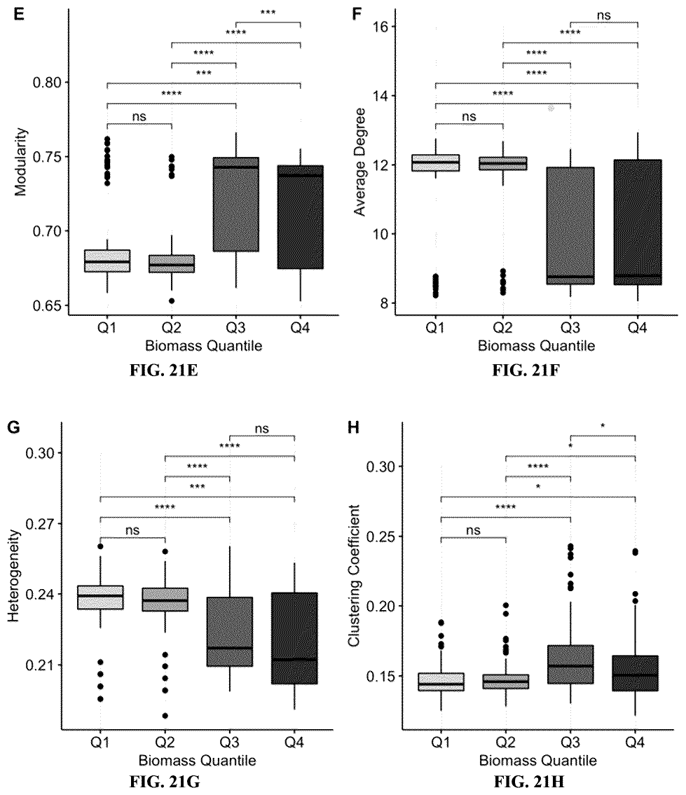

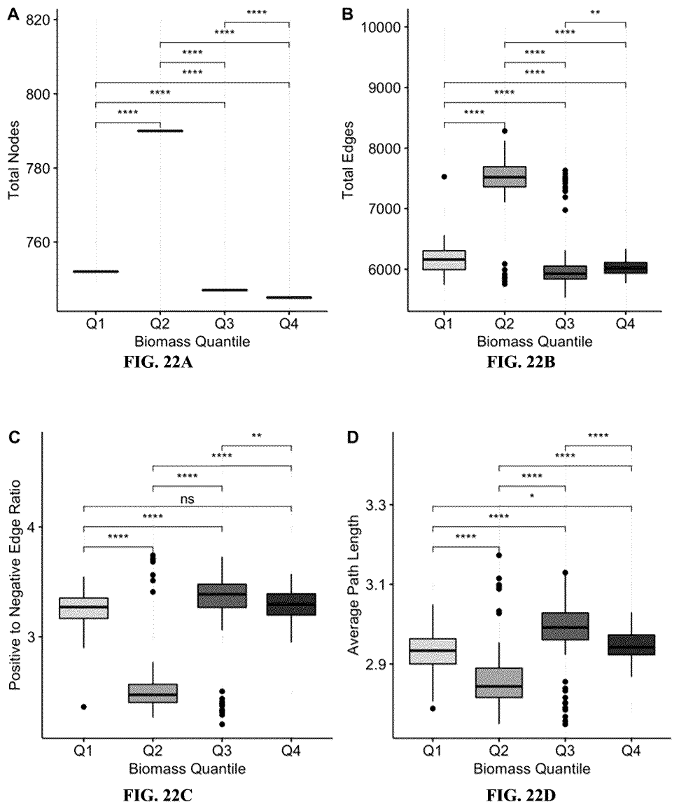

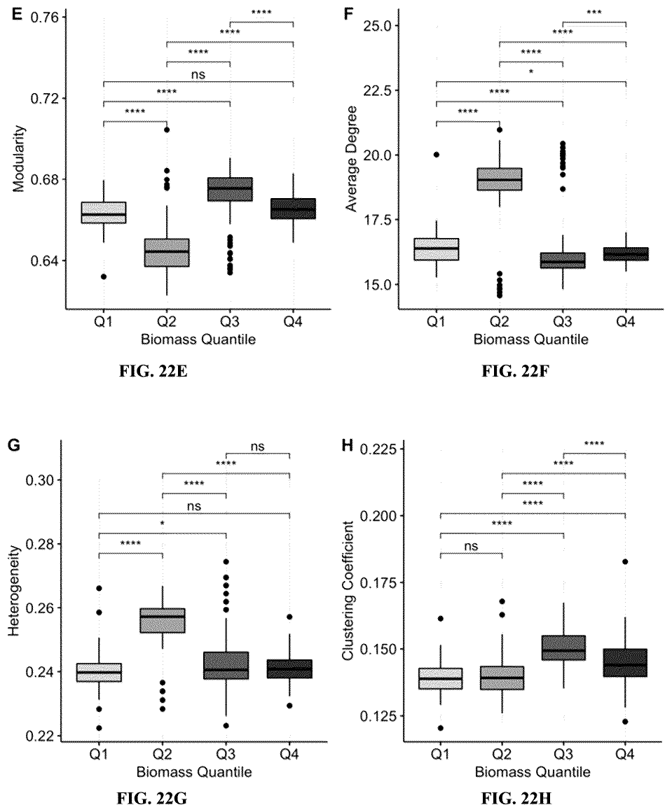

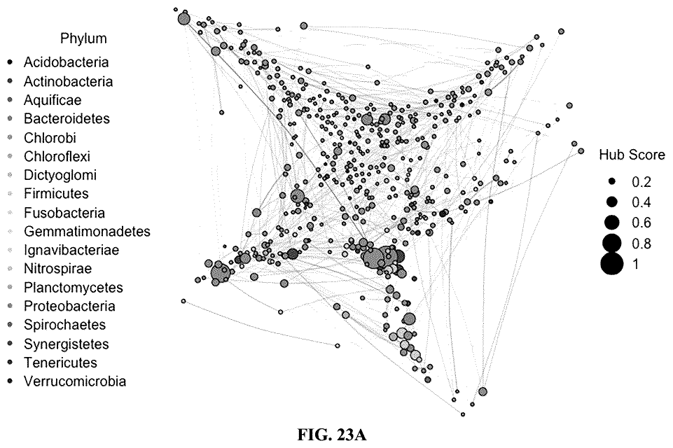

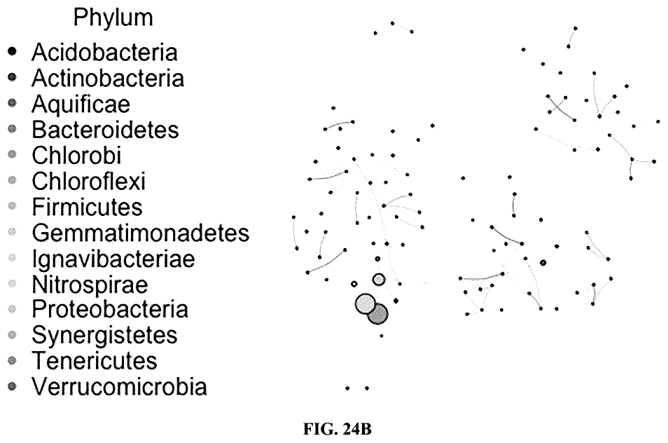

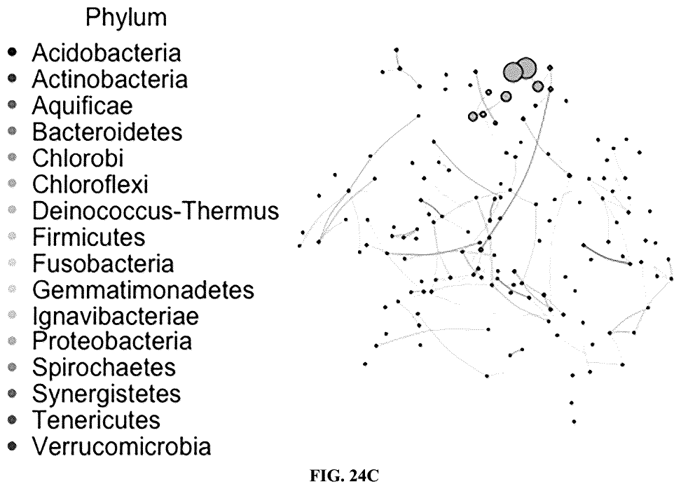

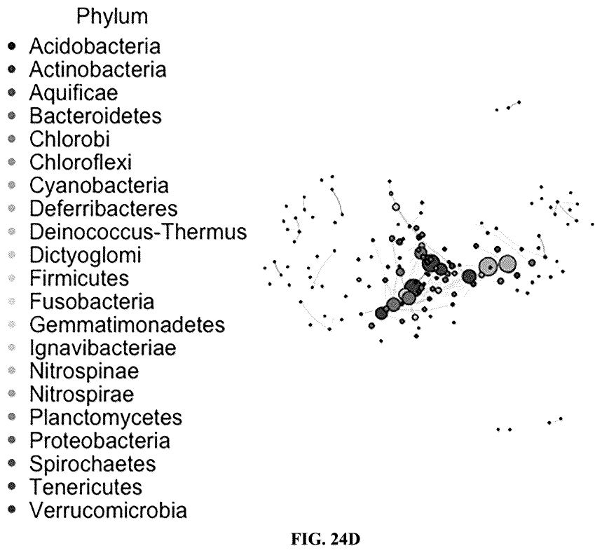

Using gametophytes from all four populations (AQ, CI, CP, and LC), co-occurrence networks were constructed of the microbial community with taxa classified at the order and family levels (FIG. 24A-FIG. 24D, FIG. 17A-FIG. 17D). Significant differences across populations were determined for the following network topology measures: total nodes, total edges, ratio of positive to negative edges, average path length, modularity, average degree, heterogeneity, and clustering coefficient (FIG. 15A-FIG. 15H and FIG. 16). All topology measures had a significant difference overall. Pairwise comparisons were significant for all combinations for total nodes, total edges, average degree, and clustering coefficient. For the remaining measures, most combinations were significantly different except for the following: ratio of positive to negative edges and modularity for the AQ and CI populations, average path length for the CI and CP populations, and heterogeneity for the AQ and CP populations. In addition to differences among network topology measures, the four populations also had distinct hub taxa. In general, the LC population had the greatest number of hub taxa with a score over 0.5. Of those identified, only two taxa overlapped between populations: Chthoniobacterales was shared between the AQ and CP populations and Cryptosporangiaceae was shared between the AQ and LC populations (Table 15).

### Modeling Community Assembly Patterns Using the Zeta Diversity Metric.

To determine whether community assembly patterns differed between low- and high biomass outcomes, zeta diversity was used to help determine the relative likelihoods of niche differentiated (non-random) and stochastic (random) processes of community assembly for kelp microbiomes found using either low- or high biomass individuals. Due to the reduced number of individuals in the AQ, CI, and CP populations, this analysis was run on the biomass quantiles from the LC population alone. In order to model community assembly patterns and determine the degree to which microbial communities were randomly structured, the zeta diversity metric was used. This metric quantifies the number of species shared between any number of sites. Zeta order refers to the number of sites being considered at a time when calculating their compositional overlap. As zeta order increases in size, the value of zeta diversity becomes increasingly influenced by more common species, and the decline in the number of shared species is modeled as an exponential or power-law regression. The relative likelihoods of an exponential versus power-law model of zeta diversity are associated with the respective relative likelihoods of a stochastic (random) versus niche-differentiated (non-random) model of community assembly. For this study, the microbiome of each unique gametophyte was considered a “site.” Abundance counts were first converted to presence (1) and absence (0) scores. Zeta decline was modeled using the ‘zetadiv’ package in R. Comparison of AIC scores was used to determine the best fitting model (exponential versus power-law regression) and the likely method of community assembly. Common species were shared between a higher number of sites while rare species were shared between fewer. Consequently, analyses were done for zeta orders 3, 5, 10, 20, and 50 at the species level to investigate the contribution of rare (lower zeta orders) versus common (higher zeta orders) species to compositional change. Analyses were also done at the class, order, family, and genus levels for zeta order 50 to determine if community assembly patterns differed between taxonomic levels.

### Community Assembly of Gametophyte Microbial Community was Niche-Driven Across Biomass Outcomes.

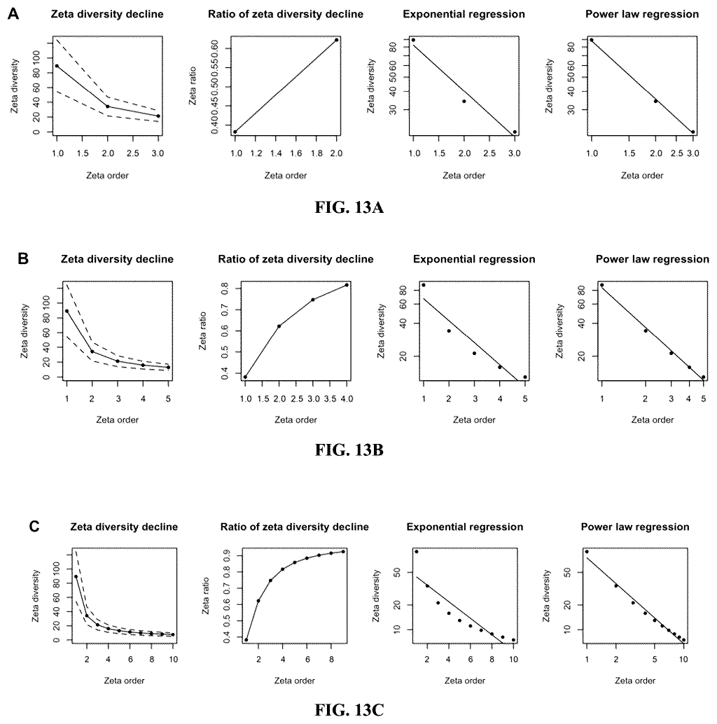

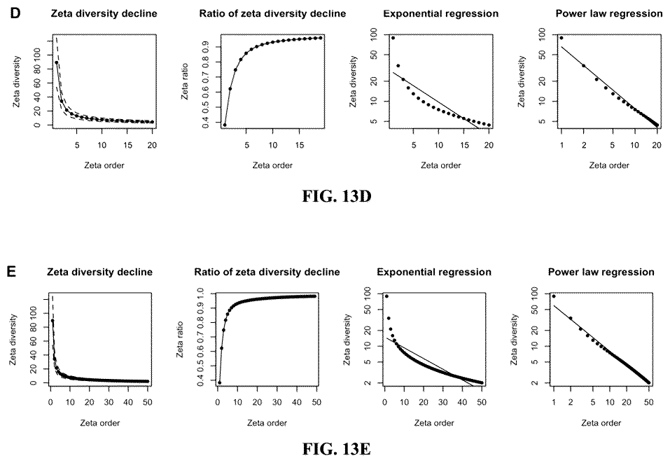

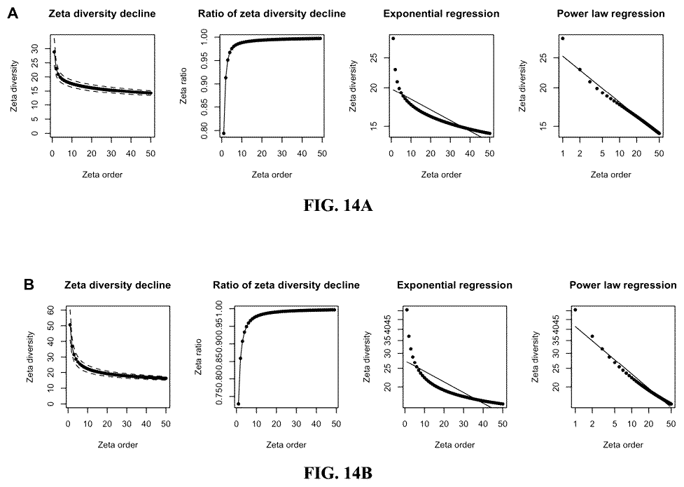

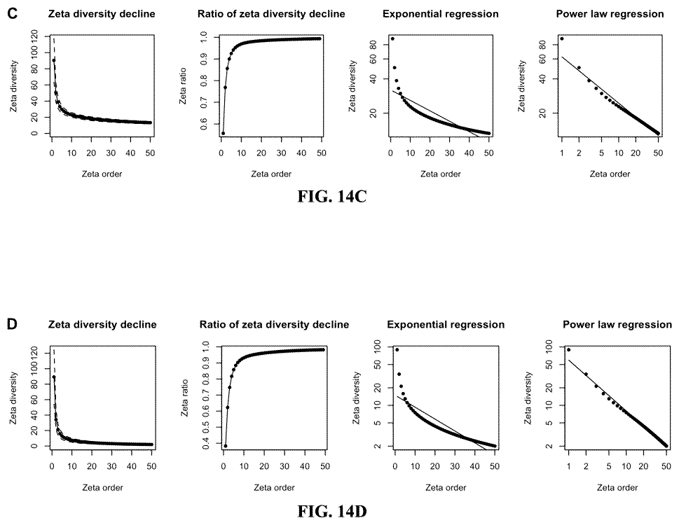

Zeta diversity, the number of shared species between three or more sites, was used to model community assembly patterns and to determine whether they were driven by stochastic (random) or niche-driven (non-random) mechanisms. Zeta order refers to the number of sites included in this measure. Here, sites refer to gametophyte microbiome samples. To understand the contribution of rare and common taxa to compositional change, zeta diversity analyses were calculated at zeta orders 3, 5, 10, 20, and 50. For gametophytes that became high biomass sporophytes, zeta diversity decline of microbial communities followed a power-law regression for all zeta orders (FIG. 13A-FIG. 13E). A similar pattern was found for all gametophyte microbial communities regardless of biomass outcome. To determine whether community assembly patterns varied across taxonomic levels, analyses with zeta order 50 were calculated at the class, order, family, and genus levels (FIG. 14A-FIG. 14D). All taxonomic levels demonstrated niche-driven assembly patterns across biomass outcomes.

### DISCUSSION

Analyses of the microbial co-occurrence network topology in gametophytes cultures across biomass outcomes revealed that several features predicted sporophyte yield. Clustering coefficient and the ratio of positive to negative edges were significant factors for the predictive modeling of sporophyte biomass when looking at gametophyte microbial networks classified at the class, order, family, and genus levels. At the species level, larger clustering coefficient values, which are associated with highly complex communities and strong microbe-microbe interactions, had a profoundly high likelihood of increased biomass. Densely connected subnetworks were associated with improved growth in M. pyrifera. Although topological analysis does not offer insight on the mechanisms behind this impact, higher clustering coefficients indicate greater cooperation that benefits the host. Likewise, a higher ratio of positive to negative edges associated with increased biomass indicates less competition between taxa that detract from host health and performance. At the order and family levels, increased heterogeneity, indicating more variation in the number of connecting edges per node, shows that when the edge connections of a network were concentrated on a small number of taxa there was a growth benefit to the host. In other words, it was beneficial to have few hub taxa, with dense connections to other members of the community, dominating associations across the network.

Hub taxa were different for M. pyrifera gametophytes that became low- and high biomass sporophytes. Hub microbes impact the colonization and abundance of other bacteria. They also impact host physiology, including host metabolism, which indirectly impacts what microbial species are present. Hub microbes from low-biomass hosts are inefficient at recruiting bacteria that provide the greatest growth benefit to the host. Consequently, this relationship is exploited to recruit beneficial microbes at the early stage of seaweeds and increase growth. In particular, the addition of taxa from the genera Wenyingzhuangia and Pedobacter or the addition of species mucus bacterium 80 and Marinomonas brasilensis to inoculates at the early life stage of M. pyrifera, recruit other beneficial bacteria and induce a growth-promoting benefit. Mesorhizobium, which is a prime candidate for growth-promoting inoculants in M. pyrifera, does not have negative associations with Wenyingzhuangia nor Pedobacter. When bacteria from these three genera are included in a growth-promoting inoculant, they do not compete with each other and even provide a synergistic effect. This is a promising finding given that perturbance and removal of hub taxa have negative cascading effects throughout a microbial community and decrease stability overall. The genera that Mesorhizobium does not co-occur with (Aquamarina, Sneathiella, Pseudohaliea, and Saccharospirillum) are not hub taxa; taxa from these genera directly compete with, or disrupt the efficacy of, a Mesorhizobium inoculation.

Network dynamics vary by population. This is likely a consequence of diverse taxa inhabiting M. pyrifera individuals from different populations, perhaps driven by genetic diversity of kelp gametophytes.

Zeta diversity analysis revealed that the microbial community assembled in a niche-driven manner when conglomerated to the class, order, family, genus, and species levels and that this was consistent across all biomass outcomes. At the species level, rare and common species similarly contributed to this assembly pattern. The community was competitively structured and assembly patterns were not a driving factor in the difference between biomass yields for M. pyrifera cultivars. Inoculants are designed in a way that does not compete with established niches so that they persist in the context of the native microbial community.

In conclusion, network dynamics and community assembly patterns of microbial communities were analyzed for cultivated M. pyrifera gametophytes and these characteristics were compared with sporophyte performance to identify features associated with increased biomass. Network dynamics and hub taxa of microbial communities at the gametophyte stage were a driving force in biomass outcomes at the sporophyte stage. In addition, microbial communities assembled in a niche-driven manner across all biomass outcomes. When designing inoculants to increase the biomass yield of M. pyrifera cultivars, avoiding competition with hub taxa identified here increases long-term efficacy. Introduction of desired hub taxa at the gametophyte stage induced the recruitment of other beneficial bacteria and shaped the overall community in a more precise manner. Genome sequencing of hub taxa elucidates functional pathways, and genome-wide association studies identify genetic factors of M. pyrifera that impact recruitment of these taxa. Incorporating analyses of the host genome is particularly exciting for growth-promoting applications discussed here, as the impact that host genotype has on the overall microbial community is strong when focused on hub microbes. Finally, inoculation trials are performed to track long-term efficacy, change in desired trait outcomes, and impacts on network structure. Altogether, the present disclosure supports the use of growth-promoting microbial inoculants in M. pyrifera cultivars and the seaweed-based biofuel industry.

Table 11 presents a summary of network topology factors, recorded for LC gametophytes (n=308) with bacteria classified at four taxonomic levels: order, family, genus, and species. LC gametophytes were divided into four biomass quantiles and a summary of all data is presented here. For each taxonomic level, 50 individuals were randomly sampled from each quantile 100 times to create representative networks. (*) Used in regression model.

| Taxonomic | Network | | | | |
|---|---|---|---|---|---|
| Level | Topology Factor | Q1 | Q2 | Q3 | Q4 |
| Order | Total Nodes | 60.23 ± 12.32 | 55.06 ± 11.44 | 48.31 ± 10.59 | 52.37 ± 11.20 |
| Order | Total Edges | 76.92 ± 26.58 | 65.08 ± 28.91 | 61.47 ± 22.92 | 69.15 ± 24.0 |
| Order | Positive Edges | 68.47 ± 21.52 | 58.48 ± 22.90 | 56.55 ± 18.68 | 63.35 ± 20.41 |
| Order | Negative Edges | 8.450 ± 5.529 | 6.600 ± 6.291 | 4.920 ± 4.598 | 5.800 ± 3.975 |
| Order | Positive/Negative | 13.38 ± 11.52 | 17.12 ± 12.08 | 14.25 ± 11.42 | 17.99 ± 11.92 |
| | Edge Ratio* | | | | |
| Order | Positive/Total | 0.9055 ± 0.05072 | 0.9188 ± 0.05046 | 0.9381 ± 0.05344 | 0.9269 ± 0.03634 |
| | Edge Ratio | | | | |
| Order | Negative/Total | 0.09450 ± 0.05072 | 0.08124 ± 0.05046 | 0.06192 ± 0.05344 | 0.07309 ± 0.03634 |
| | Edge Ratio | | | | |
| Order | Average Path | 3.927 ± 0.8294 | 3.498 ± 1.236 | 3.401 ± 0.8584 | 3.372 ± 0.8062 |
| | Length* | | | | |
| Order | Modularity* | 0.7755 ± 0.03852 | 0.7723 ± 0.04621 | 0.7672 ± 0.03230 | 0.7398 ± 0.03647 |
| Order | Average Degree | 2.468 ± 0.4588 | 2.259 ± 0.5351 | 2.456 ± 0.4492 | 2.557 ± 0.4296 |
| Order | Heterogeneity* | 0.3071 ± 0.02037 | 0.3040 ± 0.02499 | 0.3245 ± 0.02405 | 0.3266 ± 0.02673 |
| Order | Clustering | 0.1323 ± 0.07696 | 0.1082 ± 0.07814 | 0.1555 ± 0.08380 | 0.1365 ± 0.07386 |
| | Coefficient* | | | | |
| Family | Total Nodes | 151.8 ± 6.495 | 150.2 ± 7.113 | 138.6 ± 4.467 | 143.0 ± 8.932 |
| Family | Total Edges | 264.6 ± 76.85 | 262.6 ± 68.98 | 226.3 ± 32.54 | 247.1 ± 69.43 |
| Family | Positive Edges | 231.0 ± 54.07 | 237.0 ± 50.76 | 206.4 ± 24.56 | 221.4 ± 47.65 |
| Family | Negative Edges | 33.58 ± 23.33 | 25.58 ± 18.97 | 19.88 ± 9.18 | 25.67 ± 22.51 |
| Family | Positive/Negative | 8.682 ± 2.946 | 12.39 ± 5.869 | 11.37 ± 2.941 | 12.29 ± 5.436 |
| | Edge Ratio* | | | | |
| Family | Positive/Total | 0.8844 ± 0.04253 | 0.9115 ± 0.03801 | 0.9143 ± 0.02235 | 0.9083 ± 0.04703 |
| | Edge Ratio | | | | |
| Family | Negative/Total | 0.1156 ± 0.04253 | 0.08852 ± 0.03801 | 0.08569 ± 0.02235 | 0.09175 ± 0.04703 |
| | Edge Ratio | | | | |
| Family | Average Path | 5.375 ± 1.038 | 5.180 ± 0.9200 | 5.069 ± 0.5308 | 4.990 ± 0.7665 |
| | Length | | | | |
| Family | Modularity* | 0.8421 ± 0.03459 | 0.8428 ± 0.03151 | 0.8202 ± 0.01752 | 0.8254 ± 0.02740 |
| Family | Average Degree | 3.453 ± 0.8370 | 3.466 ± 0.7291 | 3.258 ± 0.3687 | 3.415 ± 0.7030 |
| Family | Heterogeneity* | 0.2462 ± 0.02006 | 0.2464 ± 0.01579 | 0.2488 ± 0.01717 | 0.2563 ± 0.01563 |
| Family | Clustering | 0.1800 ± 0.02530 | 0.1781 ± 0.02741 | 0.1613 ± 0.02421 | 0.1651 ± 0.02953 |
| | Coefficient* | | | | |
| Genus | Total Nodes | 476.0 ± 0.000 | 484.0 ± 0.000 | 461.0 ± 0.000 | 471.0 ± 0.000 |
| Genus | Total Edges | 2739 ± 335.6 | 2855 ± 238.9 | 2242 ± 377.8 | 2324 ± 417.6 |
| Genus | Positive Edges | 2151 ± 201.4 | 2228 ± 148.6 | 1813 ± 229.4 | 1908 ± 252.3 |
| Genus | Negative Edges | 588.5 ± 137.5 | 626.9 ± 95.46 | 428.9 ± 152.4 | 416.5 ± 170.4 |
| Genus | Positive/Negative | 3.864 ± 0.9146 | 3.643 ± 0.6006 | 4.534 ± 0.9049 | 5.057 ± 1.219 |
| | Edge Ratio* | | | | |
| Genus | Positive/Total | 0.7887 ± 0.0307 | 0.7820 ± 0.0208 | 0.8139 ± 0.0334 | 0.8273 ± 0.0386 |
| | Edge Ratio | | | | |
| Genus | Negative/Total | 0.2113 ± 0.03066 | 0.2180 ± 0.02084 | 0.1861 ± 0.03337 | 0.1727 ± 0.03861 |
| | Edge Ratio | | | | |
| Genus | Average Path | 3.093 ± 0.1586 | 3.088 ± 0.1125 | 3.289 ± 0.1860 | 3.253 ± 0.2052 |
| | Length | | | | |
| Genus | Modularity | 0.6893 ± 0.02833 | 0.6817 ± 0.01986 | 0.7260 ± 0.03322 | 0.7173 ± 0.03542 |
| Genus | Average Degree | 11.51 ± 1.410 | 11.80 ± 0.9872 | 9.727 ± 1.639 | 9.869 ± 1.773 |
| Genus | Heterogeneity* | 0.2338 ± 0.01620 | 0.2365 ± 0.01200 | 0.2226 ± 0.01733 | 0.2199 ± 0.01909 |
| Genus | Clustering | 0.1472 ± 0.01207 | 0.1478 ± 0.01174 | 0.1623 ± 0.02524 | 0.1547 ± 0.02265 |
| | Coefficient* | | | | |
| Species | Total Nodes | 752.0 ± 0.000 | 790.0 ± 0.000 | 747.0 ± 0.000 | 745.0 ± 00.00 |
| Species | Total Edges | 6160 ± 245.1 | 7448 ± 483.9 | 6102 ± 526.3 | 6027 ± 141.1 |
| Species | Positive Edges | 4709 ± 159.8 | 5327 ± 241.6 | 4648 ± 240.1 | 4620 ± 108.8 |
| Species | Negative Edges | 1451 ± 109.3 | 2121 ± 258.0 | 1455 ± 297.4 | 1407 ± 60.32 |
| Species | Positive/Negative | 3.256 ± 0.1609 | 2.548 ± 0.3143 | 3.269 ± 0.3725 | 3.289 ± 0.1352 |
| | Edge Ratio | | | | |
| Species | Positive/Total | 0.7646 ± 0.009726 | 0.7163 ± 0.02040 | 0.7636 ± 0.02470 | 0.7666 ± 0.007416 |
| | Edge Ratio | | | | |
| Species | Negative/Total | 0.2354 ± 0.009726 | 0.2837 ± 0.02040 | 0.2364 ± 0.02470 | 0.2334 ± 0.007416 |
| | Edge Ratio | | | | |
| Species | Average Path | 2.933 ± 0.04780 | 2.861 ± 0.07925 | 2.980 ± 0.08240 | 2.948 ± 0.03552 |
| | Length | | | | |
| Species | Modularity | 0.6631 ± 0.007506 | 0.6455 ± 0.01266 | 0.6721 ± 0.01332 | 0.6652 ± 0.007132 |
| Species | Average Degree | 16.38 ± 0.6519 | 18.86 ± 1.225 | 16.34 ± 1.409 | 16.18 ± 0.3787 |
| Species | Heterogeneity | 0.2400 ± 0.005982 | 0.2554 ± 0.007691 | 0.2430 ± 0.009911 | 0.2405 ± 0.004910 |
| Species | Clustering | 0.1390 ± 0.005656 | 0.1398 ± 0.007559 | 0.1501 ± 0.005903 | 0.1447 ± 0.008012 |
| | Coefficient* | | | | |

Table 12 includes a summary of odds ratio values for network topology factors (p<0.01) used in proportional odds logistic regression (POLR) models.

| | | Positive to | | | Average |
|---|---|---|---|---|---|
| | Clustering | Negative | Hetero- | Modu- | Path |
| | Coefficient | Edge Ratio | geneity | larity | Length |
| Order | 3.52e+3 | 1.04 | 1.00e+9 | 5.07e−8^{a} | 5.73e−1^{a} |
| Family | 2.33e−6^{a} | 1.22 | 6.34e+9 | 2.22e−10^{a} | |
| Genus | 2.46e−8^{a} | 4.65 | NS | | |
| Species | 1.60e+37 | | | | |

Separate models were built for the order, family, genus, and species taxonomic levels. Factors to include for each model were determined by best fit, and blanks indicate that a factor was not used in the model. For example, at the species level, Biomass Quantile˜Clustering Coefficient. ‘NS’ signifies that although used in the model, the factor was not a significant predictor of biomass.

The odds ratio values are presented here. For ease of interpretation, the reciprocal for values with negative exponents was calculated to represent how “less likely” the odds of increased biomass were with each one unit increase in the corresponding network topology factor, and is referenced this way in the main text. Values with positive exponents are interpreted as that much “more likely” to have increased biomass with each one unit increase in the corresponding network topology factor.

Table 13 presents a summary of p-value and odds ratio values for network topology factors used in a proportional odds logistic regression (POLR) model. Models were as follows: (Order) Biomass Quantile Positive to Negative Edge Ratio+Average Path Length+Modularity+Heterogeneity+Clustering Coefficient, (Family) Biomass Quantile˜Positive to Negative Edge Ratio+Modularity+Heterogeneity+Clustering Coefficient, (Genus) Biomass Quantile˜Positive to Negative Edge Ratio+Heterogeneity+Clustering Coefficient, and (Species) Biomass Quantile˜Clustering Coefficient.

| Taxonomic | | | Odds |
|---|---|---|---|
| Level | Network Topology Factor | P-Value | Ratio |
| Order | Clustering Coefficient | 2.64e−4 | 3.52e+3 |
| Order | Positive to Negative Edge Ratio | 3.10e−3 | 1.04 |
| Order | Heterogeneity | 2.54e−5 | 1.00e+9 |
| Order | Modularity | 7.40e−9 | 5.07e−8 |
| Order | Average Path Length | 2.16e−4 | 5.73e−1 |
| Family | Clustering Coefficient | 3.13e−3 | 2.33e−6 |
| Family | Positive to Negative Edge Ratio | 4.67e−10 | 1.22 |
| Family | Heterogeneity | 5.69e−3 | 6.34e+9 |
| Family | Modularity | 3.92e−5 | 2.22e−10 |
| Genus | Clustering Coefficient | 8.08e−3 | 2.46e−8 |
| Genus | Positive to Negative Edge Ratio | 3.30e−10 | 4.65 |
| Species | Clustering Coefficient | 1.84e−10 | 1.60e+37 |

Table 14 includes hub taxa with a Kleinberg's centrality score ofover 0.5.

| Taxonomic Level | Taxa | Hub Score | Biomass Group |
|---|---|---|---|
| Order | Desulfovibrionales | 1 | High |
| Order | Nitrospinales | 0.99 | High |
| Family | Magnetococcaceae | 1 | High |
| Family | Beijerinckiaceae | 0.95 | High |
| Family | Holosporaceae | 0.81 | High |
| Genus | Wenyingzhuangia | 1 | High |
| Genus | Pedobacter | 0.89 | High |
| Species | mucus bacterium 80 | 1 | High |
| Species | Marinomonas | 0.59 | High |
| | brasilensis | | |
| Order | Frankiales | 1 | Low |
| Order | Kineosporiales | 0.89 | Low |
| Family | Veillonellaceae | 1 | Low |
| Family | Archangiaceae | 0.99 | Low |
| Family | Burkholderiaceae | 0.89 | Low |
| Family | Clostridiaceae 1 | 0.85 | Low |
| Genus | Marixanthomonas | 1 | Low |
| Genus | Magnetococcus | 0.91 | Low |
| Genus | Epibacterium | 0.84 | Low |
| Genus | alpha proteobacterium | 0.57 | Low |
| | PWB3^{a} | | |
| Genus | Collinsella | 0.53 | Low |
| Species | Kordiimonas lacus | 1 | Low |
| Species | Methylosinus | 0.87 | Low |
| | trichosporium | | |
| Species | alpha proteobacterium | 0.74 | Low |
| | SAORIC-651 | | |
| Species | Stappia taiwanensis | 0.62 | Low |
| Species | marine bacterium | 0.55 | Low |
| | VA011 | | |

^{a}The SILVA taxonomy database is manually curated and shown to have guide tree errors. This species appears to have been incorrectly classified as a genus.

Table 15 describes hub taxa with a Kleinberg's centrality score of over 0.5 for M. pyrifera gametophytes from all four populations (AQ, CI, CP, and LC). Representative networks were generated for the microbial communities of each population. Taxa were then given a score to quantify their role as hub taxa. Taxa from the order and family levels that scored over 0.5 are recorded here. *,+ denotes hub taxa found in more than one population with a score over 0.5.

| Taxonomic | | Hub | Popu- |
|---|---|---|---|
| Level | Taxa | Score | lation |
| Order | NIASMIV (Gammaproteobacteria) | 1 | AQ |
| Order | Chthoniobacterales⁺ | 0.94 | AQ |
| Order | Chlorobiales | 1 | CI |
| Order | Ignavibacteriales | 0.56 | CI |
| Order | Bifidobacteriales | 1 | CP |
| Order | Myxococcales | 0.95 | CP |
| Order | Chthoniobacterales⁺ | 0.83 | CP |
| Order | Frankiales | 1 | LC |
| Order | Subsection III (Cyanobacteria) | 0.95 | LC |
| Order | Kineosporiales | 0.87 | LC |
| Order | Subsection IV (Cyanobacteria) | 0.79 | LC |
| Order | Micromonosporales | 0.62 | LC |
| Order | Solirubrobacterales | 0.56 | LC |
| Family | Iamiaceae | 1 | AQ |
| Family | Cryptosporangiaceae* | 0.91 | AQ |
| Family | Micrococcaceae | 0.77 | AQ |
| Family | Chlorobiaceae | 1 | CI |
| Family | Ignavibacteriales Incertae Sedis | 0.99 | CI |
| Family | Sphingomonadaceae | 0.54 | CI |
| Family | Ectothiorhodospiraceae | 1 | CP |
| Family | Granulosicoccaceae | 0.92 | CP |
| Family | Family I (Cyanobacteria, Subsection III) | 1 | LC |
| Family | Kineosporiaceae | 0.97 | LC |
| Family | Cryptosporangiaceae* | 0.96 | LC |
| Family | Family I (Cyanobacteria, Subsection IV) | 0.92 | LC |
| Family | Micromonosporaceae | 0.72 | LC |
| Family | Moritellaceae | 0.70 | LC |
| Family | Bdellovibrionaceae | 0.69 | LC |
| Family | Caedibacter caryophilus group (Rickettsiales) | 0.63 | LC |
| Family | Mycobacteriaceae | 0.62 | LC |
| Family | Staphylococcaceae | 0.61 | LC |

Table 16 describes significant differences between network features across biomass outcomes and resulting p-values for Kruskal-Wallis rank sum test comparing network features across all biomass quantiles. Results were recorded for networks built with bacteria classified at

| | P-Value | | | |
|---|---|---|---|---|
| Network Topology Factor | Order | Family | Genus | Species |
| Total Nodes | 3.743e−15 | 2.2e−16 | 2.2e−16 | 2.2e−16 |
| Total Edges | 6.259e−07 | 7.089e−09 | 2.2e−16 | 2.2e−16 |
| Positive to Negative | 3.603e−05 | 9.052e−11 | 2.051e−15 | 2.2e−16 |
| Edge Ratio | | | | |
| Average Path Length | 1.528e−05 | 0.0001952 | 1.069e−10 | 2.2e−16 |
| Modularity | 1.049e−10 | 8.513e−14 | 2.2e−16 | 2.2e−16 |
| Average Degree | 0.000253 | 0.002852 | 1.916e−14 | 2.2e−16 |
| Heterogeneity | 4.2e−13 | 4.324e−05 | 2.947e−10 | 2.2e−16 |
| Clustering Coefficient | 0.0002311 | 5.485e−07 | 7.147e−07 | 2.2e−16 |

Table 17 describes sample size and number of taxa that were used in network construction for all four biomass quantiles from the LC population.

| | Biomass Quantile | | | | | | | |
|---|---|---|---|---|---|---|---|---|
| | Q1 | | Q2 | | Q3 | | Q4 | |
| Taxonomic | Sample | Number | Sample | Number | Sample | Number | Sample | Number |
| Level | Size | of Taxa | Size | of Taxa | Size | of Taxa | Size | of Taxa |
| Order | 77 | 82 | 78 | 81 | 76 | 74 | 77 | 78 |
| Family | 77 | 163 | 78 | 163 | 76 | 156 | 77 | 162 |
| Genus | 77 | 476 | 78 | 484 | 76 | 461 | 77 | 471 |
| Species | 77 | 752 | 78 | 790 | 76 | 747 | 77 | 745 |

### REFERENCES

Agler M T, Ruhe J, Kroll S, Morhenn C, Kim S T, Weigel D, et al. Microbial Hub Taxa Link Host and Abiotic Factors to Plant Microbiome Variation. PLoS Biol. 2016; 14(1):e1002352.
Aitchison, J. 1986. The statistical analysis of compositional data. J. Roy. Stat. So.c B Met. 44:139-77.
Anderson, M. J. & Walsh, D. C. I. 2013. PERMANOVA, ANOSIM, and the Mantel test in the face of heterogeneous dispersions: What null hypothesis are you testing? Ecol. Monogr. 83:557-74.
ARPA-E. 2021. Mariner Annual Review 2021. U.S. Department of Energy.
Awany, D., Allah, I., Dalvie, S., Hemmings, S., Mwaikono, K. S., Thomford, N. E., Gomez, A., Mulder, N. & Chimusa, E. R. 2018. Host and Microbiome Genome-Wide Association Studies: Current State and Challenges. Front. Genet. 9:637.
Bakhshandeh E, Rahimian H, Pirdashti H, Nematzadeh G A. Evaluation of phosphate-solubilizing bacteria on the growth and grain yield of rice (Oryza sativa L.) cropped in northern Iran. J Appl Microbiol. 2015; 119(5):1371-82.
Bannar-Martin K H, Kremer C T, Ernest S K M, Leibold M A, Auge H, Chase J, et al. Integrating community assembly and biodiversity to better understand ecosystem function: the Community Assembly and the Functioning of Ecosystems (CAFE) approach. Ecol Lett. 2018; 21(2):167-80.
Bellona. 2017. Factsheet Pros and Cons Seaweed for Biofuel. Bellona Europa.
Bengtsson-Palme J, Hartmann M, Eriksson K M, Pal C, Thorell K, Larsson D G J, et al. metaxa2: improved identification and taxonomic classification of small and large subunit rRNA in metagenomic data. Mol Ecol Resour. 2015; 15(6):1403-14.
Benjelloun, I., Thami Alami, I., El Khadir, M., Douira, A. & Udupa, S. M. 2021. Co-Inoculation of Mesorhizobium ciceri with Either Bacillus sp. or Enterobacter aerogenes on Chickpea Improves Growth and Productivity in Phosphate-Deficient Soils in Dry Areas of a Mediterranean Region. Plants (Basel) 10:571.
Bisen K, Keswani C, Mishra S, Saxena A, Rakshit A, Singh H. Unrealized potential of seed biopriming for versatile agriculture. Nutrient use efficiency: from basics to advances: Springer; 2015. p. 193-206.
Blondel V D, Guillaume J-L, Lambiotte R, Lefebvre E. Fast unfolding of communities in large networks. Journal of Statistical Methods: Theory and Experiment. 2008; 2008(10):P10008.
Bulgarelli D, Schlaeppi K, Spaepen S, Van Themaat E V L, Schulze-Lefert P. Structure and functions of the bacterial microbiota of plants. Ann Rev Plant Biol. 2013; 64:807-38.
Camus, C., Delgado, R., Olivera-Nappa, Á., Leyton, C. & H. Buschmann, A. 2016. Scaling up bioethanol production from the farmed brown macroalga Macrocystis pyrifera in Chile. Biofuel Bioprod. Bior. 10:673-85.
Chen S, Zhou Y, Chen Y, Gu J. fastp: an ultra-fast all-in-one FASTQ preprocessor. Bioinformatics. 2018; 34(17):i884-i90.
Cornell C, Kokkoris V, Richards A, Horst C, Rosa D, Bennett J A, et al. Do Bioinoculants Affect Resident Microbial Communities? A Meta-Analysis. Frontiers in Agronomy. 2021; 3.
Csardi G, Nepusz T. The igraph software package for complex network research. InterJournal. 2006; Complex Systems:1695.
Dey, R., Pal, K. K., Bhatt, D. M. & Chauhan, S. M. 2004. Growth promotion and yield enhancement of peanut (Arachis hypogaea L.) by application of plant growth-promoting rhizobacteria. Microbiol. Res. 159:371-94.
Deysher, L. E. & Dean, T. A. 1986. Interactive effects of light and temperature on sporophyte production in the giant kelp Macrocystis pyrifera. Mar. Biol. 93:17-20.
Dittami S M, Corre E. Detection of bacterial contaminants and hybrid sequences in the genome of the kelp. PeerJ. 2017; 5:e4073.
Edgar R. Taxonomy annotation and guide tree errors in 16S rRNA databases. PeerJ. 2018; 6:e5030.
Egan S, Harder T, Burke C, Steinberg P, Kjelleberg S, Thomas T. The seaweed holobiont: understanding seaweed-bacteria interactions. FEMS Microbiol Rev. 2013; 37(3):462-76.
EIA. 2022. Biofuels explained. U.S. Energy Information Administration.
FAO. 2020. The state of world fisheries and aquaculture 2020: Sustainability in action. Food and Agriculture Organization of the United Nations, Rome, 244 pp.
Fernandes, A. D., Reid, J. N., Macklaim, J. M., McMurrough, T. A., Edgell, D. R. & Gloor, G. B. 2014. Unifying the analysis of high-throughput sequencing datasets: characterizing RNA-seq, 16S rRNA gene sequencing and selective growth experiments by compositional data analysis. Microbiome 2:15.
Gao, G., Burgess, J. G., Wu, M., Wang, S. & Gao, K. 2020. Using macroalgae as biofuel: current opportunities and challenges. Bot. Mar. 63:355-70.
García-Poza, S., Leandro, A., Cotas, C., Cotas, J., Marques, J. C., Pereira, L. & Gonçalves, A. M. 2020. The evolution road of seaweed aquaculture: Cultivation technologies and the industry 4.0. Int. J. Env. Res. Pub. He. 17:6528.
Ghaderiardakani, F., Quartino, M. L. & Wichard, T. 2020. Microbiome-Dependent Adaptation of Seaweeds Under Environmental Stresses: A Perspective. Front. Mar. Sci. 7:575228.
Gloor, G. B., Macklaim, J. M., Pawlowsky-Glahn, V. & Egozcue, J. J. 2017. Microbiome Datasets Are Compositional: And This Is Not Optional. Front Microbiol 8:2224.
Guo B, Zhang L, Sun H, Gao M, Yu N, Zhang Q, et al. Microbial co-occurrence network topological properties link with reactor parameters and reveal importance of low-abundance genera. NPJ Biofilms Microbiomes. 2022; 8(3).
Hameeda B, Harini G, Rupela O P, Wani S P, Reddy G. Growth promotion of maize by phosphate-solubilizing bacteria isolated from composts and macrofauna. Microbiol Res. 2008; 163(2):234-42.
Hui C, McGeoch M A. Zeta diversity as a concept and metric that unifies incidence-based biodiversity patterns. Am Nat. 2014; 184(5):684-94.
Imam N, Belda I, García-Jiménez B, Duehl A J, Doroghazi J R, Almonacid D E, et al. Local Network Properties of Soil and Rhizosphere Microbial Communities in Potato Plantations Treated with a Biological Product Are Important Predictors of Crop Yield. mSphere. 2021; 6(4):e0013021.
Jacob R, Harikrishnan K P, Misra R, Ambika G. Measure for degree heterogeneity in complex networks and its application to recurrence network analysis. Royal Society Open Science. 2017; 4(1):160757.
James, A. K., English, C. J., Nidzieko, N. J., Carlson, C. A. & Wilbanks, E. G. 2020. Giant kelp microbiome altered in the presence of epiphytes. Limnol. Oceanogr. Lett. 5:354-62.
Johansson M L, Alberto F, Reed D C, Raimondi P T, Coelho N C, Young M A, et al. Seascape drivers of Macrocystis pyrifera population genetic structure in the northeast Pacific. Mol Ecol. 2015; 24(19):4866-85.
Kampfer, P., Wohlgemuth, S. & Scholz, H. 2014. The Family Brucellaceae. In Rosenberg, E., DeLong, E. F., Lory, S., Stackebrandt, E. & Thompson, F. [Eds.] The Prokaryotes: Alphaproteobacteria and Betaproteobacteria. Springer Berlin Heidelberg, Berlin, Heidelberg, pp. 155-78.
Kang, Y. H., Kim, S., Choi, S. K., Lee, H. J., Chung, I. K. & Park, S. R. 2021. A comparison of the bioremediation potential of five seaweed species in an integrated fish-seaweed aquaculture system: implication for a multi-species seaweed culture. Rev. Aquacult. 13:353-64.
Kaur, G. & Reddy, M. S. 2015. Effects of Phosphate-Solubilizing Bacteria, Rock Phosphate and Chemical Fertilizers on Maize-Wheat Cropping Cycle and Economics. Pedosphere 25:428-37.
Kleinberg J. Authoritative sources in a hyperlinked environment. 1999; 46.
Kübler, J. E., Dudgeon, S. R. & Bush, D. 2021. Climate change challenges and opportunities for seaweed aquaculture in California, the United States. J. World Aquacult. Soc. 52:1069-80.
Kurtz Z, Mueller C, Miraldi E, Bonneau R. SpiecEasi: Sparse Inverse Covariance for Ecological Statistical Inference. 1.1.0 ed2021.
Lahti, L. & Shetty, S. 2017. Tools for microbiome analysis in R. 1.12.0 ed.
Langille M G, Zaneveld J, Caporaso J G, McDonald D, Knights D, Reyes J A, et al. Predictive functional profiling of microbial communities using 16S rRNA marker gene sequences. Nat Biotechnol. 2013; 31(9):814-21.
Laranjo, M., Alexandre, A. & Oliveira, S. 2014. Legume growth-promoting rhizobia: an overview on the Mesorhizobium genus. Microbiol. Res. 169:2-17.
Lata Rana, K., Kour, D., Kaur, T., Devi, R., Yadav, N., Subrahmanyam, G., Kumar, A. & Nath Yadav, A. 2020. New and Future Developments in Microbial Biotechnology and Bioengineering. Chapter 8 In Prasad, R., Gill, S. S. & Tuteja, N. [Eds.] Biotechnological applications of seed microbiomes for sustainable agriculture and environment. Elsevier, pp. 127-43.
Latombe G, McGeoch M, Nipperess D, Hui C. zetadiv: Functions to Compute Compositional Turnover Using Zeta Diversity. 1.2.0 ed2020.
Layeghifard M, Hwang D M, Guttman D S. Disentangling Interactions in the Microbiome: A Network Perspective. Trends Microbiol. 2017; 25(3):217-28.
Leibold M A, Chase J M, Ernest S K. Community assembly and the functioning of ecosystems: how metacommunity processes alter ecosystems attributes. Ecology. 2017; 98(4):909-19.
Leyton, A., Lienqueo, M. & Shene, C. 2020. Macrocystis pyrifera: Substrate for the production of bioactive compounds. J. Appl. Phycol. 32:2335-41.
Li M, O'Riordan C. The effect of clustering coefficient and node degree on the robustness of cooperation. 2013 IEEE Congress on Evolutionary Computation. 2013:2833-9.
Lin, J. D., Lemay, M. A. & Parfrey, L. W. 2018. Diverse Bacteria Utilize Alginate Within the Microbiome of the Giant Kelp. Front. Microbiol. 9:1914.
Luthringer, R., Cormier, A., Ahmed, S., Peters, A. F., Cock, J. M. & Coelho, S. M. 2014. Sexual dimorphism in the brown algae. Pers. Phycol. 1:11-25.
McGeoch M A, Latombe G, Andrew N R, Nakagawa S, Nipperess D A, Roigé M, et al. Measuring continuous compositional change using decline and decay in zeta diversity. Ecology. 2019; 100(11):e02832.
McMurdie P J, Holmes S. phyloseq: an R package for reproducible interactive analysis and graphics of microbiome census data. PLoS One. 2013; 8(4):e61217.
Meinshausen N, Buhlmann P. High-dimensional graphs and variable selection with the Lasso. The Annals of Statistics. 2006; 34(3):1436-62, 27.
Mengual, C., Schoebitz, M., Azcón, R. & Roldán, A. 2014. Microbial inoculants and organic amendment improves plant establishment and soil rehabilitation under semiarid conditions. J. Environ. Manage. 134:1-7.
Minich, J. J., Morris, M. M., Brown, M., Doane, M., Edwards, M. S., Michael, T. P. & Dinsdale, E. A. 2018. Elevated temperature drives kelp microbiome dysbiosis, while elevated carbon dioxide induces water microbiome disruption. PLoS ONE 13: e0192772.
Morales Moreira Z P, Chen M Y, Yanez Ortuno D L, Haney C H. Engineering plant microbiomes by integrating eco-evolutionary principles into current strategies. Curr Opin Plant Biol. 2022; 71:102316.
Morris M M, Haggerty J M, Papudeshi B N, Vega A A, Edwards M S, Dinsdale E A. Nearshore pelagic microbial community abundance affects recruitment success of giant kelp, Macrocystis pyrifera. Front Microbiol. 2016; 7:1800.
Navarrete, I. A., Kim, D. Y., Wilcox, C., Reed, D. C., Ginsburg, D. W., Dutton, J. M., Heidelberg, J., Raut, Y. & Wilcox, B. H. 2021. Effects of depth-cycling on nutrient uptake and biomass production in the giant kelp Macrocystis pyrifera. Renew. Sust. Energ. Rev. 141:110747.
Neushul, M. & Haxo, F. T. 1963. Studies on the Giant Kelp, Macrocystis. I. Growth of Young Plants. Am. J. Bot. 50:349-53.
NOAA. 2020. NOAA Announces Regions for First Two Aquaculture Opportunity Areas under Executive Order on Seafood. National Oceanic and Atmospheric Administration Office of Aquaculture.
O'Callaghan M. Microbial inoculation of seed for improved crop performance: issues and opportunities. Applied microbiology and biotechnology. 2016; 100(13):5729-46.
Oksanen, J., Blanchet, F. G., Friendly, M., Kindt, R., Legendre, P., McGlinn, D., Minchin, P. R., O'Hara, R. B., Simpson, G. L., Solymos, P., Stevens, M. H. H., Szoecs, E. & Wagner, H. 2020. vegan: Community Ecology Package. R package version 2.5-7 ed.
Oppliger L V, Correa J A, Faugeron S, Beltrin J, Tellier F, Valero M, et al. Sex Ratio Variation in the Lessonia Nigrescens Complex (Laminariales, Paeophyceae): Effect of Latitude, Temperature, and Marginality (1). J Phycol. 2011; 47(1):5-12.
Osborne M G, Molano G, Simons A L, Dao V, Ong B, Vong B, et al. Natural variation of Macrocystis pyrifera gametophyte germplasm culture microbiomes and applications for improving yield in offshore farms. J Phycol. Forthcoming 2023.
Osborne, M. G. & DeWeese, K. J. 2021. Understanding the metabolome and metagenome as extended phenotypes: The next frontier in macroalgae domestication and improvement. J. World Aquacult. Soc. 52:1009-30.
Pankievicz, V. C. S., Irving, T. B., Maia, L. G. S. & Ané, J. M. 2019. Are we there yet? The long walk towards the development of efficient symbiotic associations between nitrogen-fixing bacteria and non-leguminous crops. BMC Biol. 17:99.
Park, J. S., Shin, S. K., Wu, H., Yarish, C., Yoo, H. I. & Kim, J. K. 2021. Evaluation of nutrient bioextraction by seaweed and shellfish aquaculture in Korea. J. World Aquacult. Soc. 52:1118¬34.
Pfister, C. A., Altabet, M. A. & Weigel, B. L. 2019. Kelp beds and their local effects on seawater chemistry, productivity, and microbial communities. Ecology 100: e02798.
Provasoli L, editor Media and prospects for the cultivation of marine algae. Cultures and collections of algae Proc U S-Japan Conference, Hakone (ed A Watanabe & R Hattori); 1968.
Purcell-Meyerink, D., Packer, M. A., Wheeler, T. T. & Hayes, M. 2021. Aquaculture Production of the Brown Seaweeds Laminaria digitata and Macrocystis pyrifera: Applications in Food and Pharmaceuticals. Molecules 26:1306.
Qiu Z, Egidi E, Liu H, Kaur S, Singh B K. New frontiers in agriculture productivity: Optimised microbial inoculants and in situ microbiome engineering. Biotechnol Adv. 2019; 37(6):107371.
Quast C, Pruesse E, Yilmaz P, Gerken J, Schweer T, Yarza P, et al. The SILVA ribosomal RNA gene database project: improved data processing and web-based tools. Nucleic Acids Res. 2013; 41(Database issue):D590-6.
Qureshi M A, Ahmad Z A, Akhtar N, Iqbal A, Mujeeb F, Shakir M. Role of phosphate solubilizing bacteria (PSB) in enhancing P availability and promoting cotton growth. J Anim Plant Sci. 2012; 22:204-10.
R (Core Team). 2020. R: A language and environment for statistical computing. R Foundation for Statistical Computing.
Racine, P., Marley, A., Froehlich, H. E., Gaines, S. D., Ladner, I., MacAdam-Somer, I. & Bradley, D. 2021. A case for seaweed aquaculture inclusion in US nutrient pollution management. Mar. Policy 129:104506.
Ramírez-Puebla, S. T., Weigel, B. L., Jack, L., Schlundt, C., Pfister, C. A. & Mark Welch, J. L. 2022. Spatial organization of the kelp microbiome at micron scales. Microbiome 10:52.
Ramond P, Sourisseau M, Simon N, Romac S, Schmitt S, Rigaut-Jalabert F, et al. Coupling between taxonomic and functional diversity in protistan coastal communities. Environ Microbiol. 2019; 21(2):730-49.
Reed, D. C., Rassweiler, A. & Arkema, K. K. 2008. Biomass rather than growth rate determines variation in net primary production by giant kelp. Ecology 89:2493-505.

### REFERENCES

Rocha I, Ma Y, Souza-Alonso P, Vositka M, Freitas H, Oliveira R S. Seed coating: a tool for delivering beneficial microbes to agricultural crops. Front Plant Sci. 2019; 10:1357.
RStudio. 2019. RStudio: Integrated Development for R. RStudio, Inc.
Santos M S, Nogueira M A, Hungria M. Microbial inoculants: reviewing the past, discussing the present and previewing an outstanding future for the use of beneficial bacteria in agriculture. AMB Express. 2019; 9(1):1-22.
Schiel, D. R. & Foster, M. S. 2015. The biology and ecology of giant kelp forests. University of California Press, 416 pp.
Serrão, E. A. 2015. Seascape drivers of Macrocystis pyrifera population genetic structure in the northeast Pacific. Mol. Ecol. 24:4866-85.
Shade, A., Jacques, M. A. & Barret, M. 2017. Ecological patterns of seed microbiome diversity, transmission, and assembly. Curr. Opin. Microbiol. 37:15-22.
Singh D P, Singh H B, Prabha R. Microbial inoculants in sustainable agricultural productivity: Springer; 2016.
Song, X., Liu, M., Wu, D., Griffiths, B. S., Jiao, J., Li, H. & Hu, F. 2015. Interaction matters: Synergy between vermicompost and PGPR agents improves soil quality, crop quality and crop yield in the field. Appl. Soil Ecol. 89:25-34.
Team R C. R: A language and environment for statistical computing. R Foundation for Statistical Computing; 2020.
Theis, K. R., Dheilly, N. M., Klassen, J. L., Brucker, R. M., Baines, J. F., Bosch, T. C., Cryan, J. F., Gilbert, S. F., Goodnight, C. J., Lloyd, E. A., Sapp, J., Vandenkoornhuyse, P., Zilber-Rosenberg, I., Rosenberg, E. & Bordenstein, S. R. 2016. Getting the Hologenome Concept Right: an Eco-Evolutionary Framework for Hosts and Their Microbiomes. mSystems 1: e00028-16.
Timmusk S, Behers L, Muthoni J, Muraya A, Aronsson A-C. Perspectives and Challenges of Microbial Application for Crop Improvement. Front Plant Sci. 2017; 8.
Toju H, Peay K G, Yamamichi M, Narisawa K, Hiruma K, Naito K, et al. Core microbiomes for sustainable agroecosystems. Nat Plants. 2018; 4(5):247-57.
Trivedi, P., Batista, B. D., Bazany, K. E. & Singh, B. K. 2022. Plant-microbiome interactions under a changing world: responses, consequences and perspectives. New Phytol. 234:1951-59.
Ullah, S., Khan, M. Y., Asghar, H. N., Akhtar, M. J. & Zahir, A. Z. 2017. Differential response of single and co-inoculation of Rhizobium leguminosarum and Mesorhizobium ciceri for inducing water deficit stress tolerance in wheat. Ann. Microbiol. 67:739-49.
Vacher C, Hampe A, Porté AJ, Sauer U, Compant S, Morris C E. The Phyllosphere: Microbial Jungle at the Plant-Climate Interface. Ann Rev Ecol Evol Syst. 2016; 47(1):1-24.
Venables W N, Ripley B D. Modern Applied Statistics with S. Fourth ed: Springer; 2002.
Weigel, B. L. & Pfister, C. A. 2019. Successional Dynamics and Seascape-Level Patterns of Microbial Communities on the Canopy-Forming Kelps. Front. Microbiol. 10:346.
Weigel, B. L. & Pfister, C. A. 2021. Oxygen metabolism shapes microbial settlement on photosynthetic kelp blades compared to artificial kelp substrates. Environ. Microbiol. Rep. 13:176-84.

Although the invention has been described with reference to the above examples, it will be understood that modifications and variations are encompassed within the spirit and scope of the invention. Accordingly, the invention is limited only by the following claims.

What is claimed is:

1. A method of obtaining harvested seaweed having at least one desired characteristic comprising:
a) contacting a seaweed spore with a culture media comprising an inoculum, wherein the inoculum comprises an effective amount of at least one microbe;
b) growing the spore into a gametophyte;
c) outplanting the gametophyte; and
d) harvesting the seaweed.

2. The method of claim 1, wherein the harvested seaweed has at least one desired characteristic that distinguishes the harvested seaweed from non-inoculated seaweed, selected from high biomass, increased growth rate, increased yield, increased stress tolerance, increased resistance to disease, increased pathogen resistance, increased photosynthetic rate, increased tolerance of heavy metals, apical cell growth rate, disease resistance, apical cell multiplication rate, root length, root nodulation, blade length, stalk length, pneumatocyst size, pneumatocyst number, mineral content, chelation of micronutrients, heat tolerance, cold tolerance, or chlorophyll content.

3. The method of claim 1, wherein the at least one desired characteristic is high biomass.

4. The method of claim 2, wherein the non-inoculated seaweed is selected from a parent sporophyte, a sporophyte of the same variant, a sporophyte of the same gametophyte culture, a sporophyte of the same cultivar, or a sporophyte of the same genotype.

5. The method of claim 1, wherein the inoculum comprises an effective amount of at least one microbe isolated from a seaweed, one or more spores from a seaweed, or one or more seaweed gametophytes, or any combination thereof.

6. The method of claim 1, wherein the inoculum comprises an effective amount of at least one microbe isolated from a high biomass seaweed, one or more spores from a high biomass seaweed, or one or more gametophytes from a high biomass seaweed, or any combination thereof.

7. The method of claim 6, wherein the at least one microbe is selected from Marinomonas brasilensis, mucus bacterium 80, Zoogloea ramigera, Mesorhizobium species, Nitratireductor species, Aquamicrobium species, Pedobacter species, Wenyinzhuangia species, species from the order Desulfovibrionales, species from the order Nitrospinales, species from the family Magnetococcaceae, species from the family Beijerinckiaceae, species from the family Holosporaceae, species from the family Brucellaceae, species from the order Sphingomondales, and species from the order Thermoanaerobacterales; or any combination thereof.

8. The method of claim 7, wherein the inoculum comprises at least one microbe selected from Mesorhizobium species, Wenyingzhuangia species and Pedobacter species; or a variant thereof.

9. The method of claim 8, wherein the inoculum comprises at least one Mesorhizobium species or a variant thereof.

10. The method of claim 1, wherein the inoculum optionally does not contain species from the genus Labrenzia.

11. The method of claim 1, wherein the culture media comprises agriculturally acceptable excipients.

12. The method of claim 1, wherein the seaweed is Macrocystis pyrifera or a variant thereof.

13. The method of claim 1, wherein the seaweed spore in b) is a Macrocystis pyrifera spore or a variant thereof.

14. The method of claim 1, wherein the culture media comprises sterile Provasoli enriched seawater medium.

15. An agricultural formulation for an inoculant comprising a mixture of two or more bacterial species selected from Marinomonas brasilensis, mucus bacterium 80, Zoogloea ramigera, Mesorhizobium species, Nitratireductor species, Aquamicrobium species, Pedobacter species, Wenyinzhuangia species, species from the order Desulfovibrionales, species from the order Nitrospinales, species from the family Magnetococcaceae, species from the family Beijerinckiaceae, species from the family Holosporaceae, species from the family Brucellaceae, species from the order Sphingomondales, or species from the order Thermoanaerobacterales.

16. The agricultural formulation of claim 15, further comprising at least one agriculturally acceptable excipient.

17. The agricultural formulation of claim 16, wherein the agricultural formulation does not contain species from the genus Labrenzia.

18. The agricultural formulation of claim 16, wherein the agricultural formulation optionally contains species from the genus Labrenzia.

19. A method of making an inoculant comprising:
a) collecting a sporophyte from a defined geographical region;
b) collecting a spore from the sporophyte; c) analyzing at least one microbe obtained from the sporophyte;
d) growing a mature sporophyte from the spore;
e) identifying the mature sporophyte as a high biomass sporophyte;
f) culturing a microbial community of the high biomass sporophyte in biomass media;
and
g) adding at least one agriculturally acceptable excipient to the biomass media to make an inoculant.

20. The method of claim 19, wherein the high biomass sporophyte is a Macrocystis pyrifera sporophyte.

21. A method of obtaining a high biomass seaweed comprising incubating at least one spore or gametophyte with a culture media comprising the inoculant of claim 19.

22. A method of obtaining high biomass seaweed comprising:
a) releasing a spore from a sporophyte in a culture media;
b) identifying at least one bacterial species present in the culture media;
c) determining that the at least one bacterial species is associated with a high biomass seaweed;
d) growing the spore into a gametophyte;
e) outplanting the gametophyte; and
f) harvesting a high biomass seaweed.

23. The method of claim 22, wherein the high biomass seaweed is Macrocystis pyrifera.

24. The method of claim 22, wherein the culture media is sterile Provasoli enriched seawater medium.

25. The method of claim 24, wherein c) comprises modeling network features of a microbiome of the spore to determine that the at least one bacterial species is associated with a high biomass seaweed.

26. The method of claim 25, wherein the network features used are chosen from clustering coefficients, positive to negative edge ratios, modularity, heterogeneity or average path length.

27. The method of claim 1, wherein the inoculum is obtained by:
a) swabbing blades of a sporophyte to collect a sample;
b) verifying the presence of microbes in the sample;
c) culturing the microbes to form a microbial community; and
d) adding an agricultural excipient to the microbial community to form an inoculum.

28. The method of claim 1, wherein the inoculum is obtained by:
a) releasing a spore from the first sporophyte;
b) identifying microbes from the first sporophyte and from the spore;
c) producing a second sporophyte from the spore;
d) confirming the second sporophyte is a high biomass seaweed;
e) culturing the microbial community; and
f) adding an agricultural excipient to the microbial community to form an inoculum.

29. The method of claim 1, wherein the inoculum is obtained by:
a) measuring biomass of a sporophyte;
b) collecting a microbial community from the sporophyte;
c) quantifying diversity of microbial taxa in the microbial community;
d) calculating and recording clustering coefficient values of the microbial community at multiple taxonomic levels, wherein the calculating and recording clustering coefficient values comprises at least one of;
i) identifying the microbial community as having a large clustering coefficient value at the species level;
ii) identifying the microbial community as having few hub taxa with dense connections at the order and/or family level;
e) culturing the microbial community; and
f) adding an agricultural excipient to the microbial community to form an inoculum.
